(19) United States
(12) Patent Application Publication
(10) Pub. No.: US 2023/0101936 A1
(43) Pub. Date: Mar. 30, 2023

(54) LABEL-FREE FOOD ANALYSIS AND MOLECULAR DETECTION

(71) Applicant: Purdue Research Foundation, West Lafayette, IN (US)

(72) Inventors: Bartlomiej P. Rajwa, West Lafayette, IN (US); Euiwon Bae, West Lafayette, IN (US); J. Paul Robinson, West Lafayette, IN (US); Carmen Gondhalekar, West Lafayette, IN (US); Xi Wu, West Lafayette, IN (US)

(21) Appl. No.: 17/953,527

(22) Filed: Sep. 27, 2022

Related U.S. Application Data
(60) Provisional application No. 63/248,784, filed on Sep. 27, 2021.

(51) Int. Cl. G01N 21/31; G01N 33/02; G01N 33/68

(52) U.S. Cl. CPC G01N 21/31; G01N 33/02; G01N 33/6869; G01N 2021/3196

(57) ABSTRACT

The invention generally relates to methods, reagents, and substrates for detecting target analytes, especially spectroscopic techniques such as laser-induced breakdown spectroscopy (LIBS) for use in food authentication and molecular detection (e.g., when combined with later flow immunoassays (LFIA).

### RELATED APPLICATION

The present application claims the benefit of and priority to U.S. provisional patent application Ser. No. 63/248,784, filed Sep. 27, 2021, the content of which is incorporated by reference herein in its entirety.

### GOVERNMENT SUPPORT

This invention was made with government support under 59-8072-6-001 awarded by the U.S. Department of Agriculture. The government has certain rights in the invention.

### FIELD OF THE INVENTION

The invention generally relates to methods, devices, reagents, and substrates for label-free food analysis and molecular detection using laser-induced breakdown spectroscopy (LIBS). Detected molecules may include, for example, drugs, proteins, enzymes, hormones, polypeptides, or nucleic acid.

### BACKGROUND

The broadly-defined concept of food fraud includes, among other events, adulteration, substitution, dilution, tampering, misrepresentation of food, country of origin, food ingredients, overtreating, and intellectual property rights counterfeiting. Although adulteration of food products occurring in the cases of food fraud are typically performed for economic gain with no direct intent to harm the consumers physically, the use of unapproved processing methods and/or the introduction of adulterant substances compromising the integrity of the food products may have severe unintended health-related consequences. The most well-known cases include the two decades of re-occurring dioxin incidents in Europe in which poultry supply chains were affected by feed ingredients contaminated unintentionally by industrial oils. Another known example from China is the adulteration of dairy products with melamine, a nitrogen-rich organic base generally used in plastic manufacture, which was added to raise nitrogen-based tests for protein content. The contamination was first noticed in 2007 in the US because of the imported pet food affected by the end of 2008 an estimated 300 000 human victims, including six child fatalities due to kidney damage, with a further 860 infants hospitalized. The most recent case of food authentication and possibly food fraud involves mislabeling canned fish products in Subway tuna sandwiches. Currently, there is a highly publicized class-action lawsuit in the state of California claiming that Subway tuna sandwiches “are completely bereft of tuna as an ingredient.” Although Subway denies the allegations, the issue of food authentication has been broadly discussed. The food fraud incidents such as those mentioned above demonstrate that improving food integrity, testing food product authenticity, and monitoring the chemical content of the food products would not only increase consumer trust in products and brands and indirectly enhance food safety and quality.

Food production, processing, and supply are becoming globally integrated, providing unprecedented access to a continuously broadening array of products. The inevitable expansion of a service network from supplier to customer and the inclusion of global actors inherently increases the risks associated with that supply chain. The monitoring of food products also becomes more difficult in the presence of growing complexity and the growing variety of the offered goods. Verifying the integrity of the products by monitoring and fingerprinting the ingredients is a well-understood concept, but its practical implementation requires a sophisticated combination of biophysical and chemical analysis tools, statistical methods, data processing, and machine learning techniques.

Numerous biophysical methods have been proposed for label-free testing, authentication, and fingerprinting of food products. These include: chromatography, mass spectroscopy, ELISA, conventional Raman and surface-enhanced Raman scattering (SERS), Fourier transform infrared (FT-IR) spectroscopy and nuclear magnetic resonance.

Additionally, a need for rapid molecular detection and relative quantification exists. Evidence suggests that mortality in COVID-19 patients is closely associated with a cytokine storm. However, cytokine tests take an entire day of technicians' time so are not practical for rapid responses. It is well understood that production of some cytokines such as IFN-1 represents a protective component of the immune response (Cohen, 2002; Kellum et al., 2007), however, it has also been suggested that if such cytokines appear in significant levels at inappropriate times, the presence of IFN-1 enhanced proinflammatory cytokine expression resulting in fatal pneumonia (Channappanavar et al., 2019). All immune responses elicit cytokines and some elicit what is termed a cytokine storm. Because cytokine reactions are common with a variety of syndromes, attempts have been made to characterize these under the term Cytokine release syndrome (CRS)(Lee et al., 2019). With the advent of CartT therapy, and the uncreased use of monoclonal antibody therapy, there has been a recognition that there is a need for some standards to compare positive and negative cytokine results (Vessillier et al., 2020).

However with the recognition of a syndrome involving SARS-Cov-2 virus, it was evident very quickly that cytokine responses were important. (De Biasi et al., 2020; Guaraldi et al., 2020; Vacchi et al., 2020). Early reports suggested high levels of proinflammatory cytokines causing cytokine storms were responsible for pathologic inflammation (as summarized in a review by Vavet et al (Vabret et al., 2020). However, recent studies suggest that the agent that causes Covid-19, SARS-Cov-2 has the capacity to elicit a particularly serious cytokine storm in some patients (Olbei et al., 2021). Some of the earlies reports identified extremely high levels of IP-10 and MCP-3 levels (Yang et al., 2020). Other studies identified IL-6 and TNF-A were both elevated and were significant predictors of disease severity and death (Del Valle et al., 2020). Laing et all identified the main culprits as being IP-10, IL-10 and IL-6 (Laing et al., 2020).

Over the past ten years or so, a variety of techniques have been developed to measure cytokines. These include ELISA, Electrochemiluminescence, and xMAP bead-based assays. These are not easy or fast assays to perform, and there are significant challenges, such as the half-life of cytokines in serum as well as the need to attain very low levels of sensitivity in most circumstances—usually at the pg/mL level—at least for regular cytokine levels. Early assays identified the difficulty of measuring cytokines directly from serum which was seen as desirable (Finkelman & Morris, 1999). In the early 2000s comparisons were made of different assay techniques including comparison of bead-based assays to ELISA (Elshal & McCoy, 2006).

### SUMMARY

Systems and methods of the invention apply spectroscopic techniques such as laser-induced breakdown spectroscopy (LIBS) for sample classification and verification (e.g., food authentication or fingerprinting) as well as rapid molecular detection (e.g., cytokine detection to profile immune response and diagnose cytokine storms).

The use LIBS of in biological or agricultural applications is relatively rare, as the technique is typically employed in material science and basic physics research. However, recently, there has been a growing interest in LIBS due to the data's rich information content, negligible cost of individual measurement, and affordable instrumentation. LIBS is based on atomic optical emission spectroscopy, using a high-power pulse laser that ablates, atomizes, and ionizes a tiny amount of the analyte to produce a plasma plume. The generated plasma contains a mixture of atoms, ions, and free electrons from the examined material. Upon cooling of plasma, some energy is emitted, and the optical spectroscopy in the LIBS device acquires the spectral signal conveying information about the sample's elemental composition.

Over the last decade, LIBS has gained increased attention, and the review of food analysis publications that make use of LIBS shows that they are mainly centered on a component analysis (61.84%), contaminant analysis (30.53%), and detection of adulteration (7.63%). The competing methods for assessing the mineral composition and presence of inorganic impurities in food, such as atomic absorption spectroscopy, X-ray fluorescence spectrometry (XRF), ICP mass spectroscopy, are costly, produce large amounts of toxic waste, require expensive reagents, gases, and fume hoods, making portable, inexpensive implementations impossible. The described technology integrates LIBS and statistical machine learning to fingerprint and classify alpine-style cheeses, coffee, olive oil, vanilla extracts, balsamic vinegar, and potentially other food products for authentication. The system can include a bench-top or portable LIBS system, a data normalization, pre-processing and reduction algorithms, and machine-learning unsupervised and supervised classification methods. The examples show results of research on the development of LIBS-based fingerprinting will focus on three types of food products (oils, especially olive oil, dairy products with a particular emphasis on hard cheeses, and spices), but the techniques of the invention can be applied to other foods such as meat, fish, and fresh vegetables.

A need exists for a fast cytokine assay that can be effective using portable laser induced breakdown spectroscopy (LIBS) instruments. Described herein are combined lateral flow immunoassays with LIBS analysis capable of detecting and quantifying cytokine levels in 15 minutes or less. This technology would be groundbreaking not only for COVID-19, but any patients at risk of immune distress. Accumulating evidence suggests that cytokine storm syndrome (CSS) induced by the SARS-CoV-2 may be the ultimate cause of acute respiratory distress syndrome (ARDS), resulting in severe outcomes of COVID-19 and potentially death. Elevated levels of serum interleukin 6 (IL-6) and interferon gamma-induced protein 10 (IP-10) correlate with the occurrence of respiratory failure, ARDS, and adverse clinical outcomes in many COVID-19 patients. The currently available clinical cytokine tests are costly, time-consuming, expensive, and require highly trained staff to execute. There is an unmet need for affordable, robust, rapid, and sensitive tests for cytokine and chemokine levels. Disclosed techniques herein combine detection based on laser-induced breakdown spectroscopy with a lateral flow immunoassay (LIBS-LFIA) to deliver a quantitative clinical analysis platform with multiplexing capability. Lanthanide-complexed polymers (LCPs) may be selected as the labels to provide the optimal quantitative performance when sensing the signals from the test (T) lines of LFIAs. IL-6 has been successfully characterized using these methods as it is one of the most critical pro-inflammatory cytokines. The LIBS-LFIA biosensor can achieve a detection limit of 0.2298 μg/mL of IL-6 within 15 min, demonstrating superiority to several conventional methods. A new direction for LFIA design and optimization based on geometric flow control (GFC) of nitrocellulose (NC) membranes is disclosed herein, leading to increased sensitivity. This new technique enables comprehensive flow control via various membrane geometric features such as the width and the length to improve analytical performance and reduce antibody consumption. The systems and methods of the invention have many applications to bio-detection. The disclosed rapid and accurate detection of cytokines for clinical diagnosis and prognosis of COVID-19 and other pathogenic infections using LIBS is highly feasible and compatible with the POC format.

Aspects of the invention include methods for sample classification including obtaining a plurality of known samples, performing a spectroscopic analysis on the plurality of known samples to obtain an emission spectrum from each of the plurality of known samples, and processing data from the emission spectra to identify a spectral fingerprint for each of the plurality of known samples using automated feature selection.

In certain embodiments, the sample is a food sample. The sample may be selected from the group consisting of cheese, coffee, olive oil, vanilla extract, and spices. The spectroscopic analysis performed can comprise laser-induced breakdown spectroscopy (LIBS). The automated feature selection may comprise one or more machine learning classification techniques such as linear discriminant analysis (LDA), an artificial neural network (ANN), support vector machine (SVM), random forest (RF), and elastic net (ENET) regression.

One or more of the plurality of known samples may be a liquid sample and methods of the invention may further comprising depositing the liquid sample on a cellulose strip before performing the spectroscopic analysis. In certain embodiments, methods of the invention may include obtaining a test sample, performing a spectroscopic analysis on the test sample to obtain an emission spectrum from the test sample, and authenticating the test sample by comparing the emission spectra for the test sample to an expected spectral fingerprint from the spectral fingerprints for the plurality of known samples.

Processing steps may further comprise spectral baseline adjustment and correction, filtering and denoising, normalization, univariate feature filtering employing generalized linear models, multivariate feature selection and classification using regularized regression, and classification using one or more machine learning methodologies. In certain embodiments, the one or more machine learning methodologies can comprise an elastic-net feature selection model with combined LASSO and ridge penalties.

Methods of the invention may further comprise providing one or more additional data points for the plurality of known samples, wherein the processing the data from the emission spectra step includes analysis the one or more additional data points to identify a fingerprint for each the plurality of known samples comprising features selected from among the one or more additional data points along with the spectral fingerprint. The one or more additional data points may include spectra from one or more different spectroscopic technique or data from one or more biophysical testing methods.

In certain aspects, methods of the invention can include detecting molecules in a sample by providing a sample comprising a target molecule; applying the sample to a porous substrate comprising metal-conjugated capture molecules specific to the target molecule; wicking the sample along the porous substrate to concentrate target molecule bound capture molecules at a test region on the porous substrate and to concentrate unbound capture molecules at a control region on the porous substrate; performing a spectroscopic analysis on the test region and the control region to detect a concentration of the metal-conjugated capture molecules therein; and confirming presence of the target molecule in the sample based on detection of the metal-conjugated capture molecules in both the test region and the control region.

The metal-conjugated capture molecule can comprise a gold nanoparticle-conjugated antibody specific to the target molecule. The metal-conjugated capture molecule may comprise a lanthanide-conjugated antibody specific to the target molecule. In certain embodiments, the molecule may comprise a cytokine and the cytokine may comprise interleukin 6 (IL-6). In various embodiments, the target molecule may be any cytokine (e.g., see FIG. 33) or any drugs, proteins, enzymes, hormones, polypeptides, or nucleic acid. The sample may be obtained from a patient at risk of a cytokine storm.

Methods of the invention may further include quantifying an amount of metal-conjugated capture molecules concentrated at the test region using the spectroscopic analysis. The spectroscopy analysis can comprise laser-induced breakdown spectroscopy (LIBS). The porous substrate may be a nitrocellulose membrane. In certain embodiments, the confirming presence step can occur 15 minutes or less after the applying step.

### BRIEF DESCRIPTION OF THE DRAWINGS

FIG. 1 shows an exemplary portable instrument according to certain embodiments.

FIG. 2 shows an exemplary benchtop instrument according to certain embodiments.

FIG. 3 shows a diagram of an exemplary algorithm process according to certain embodiments.

FIG. 4 shows a diagram of exemplary data processes according to certain embodiments.

FIG. 5 shows LIBS analysis results of cheese using a benchtop instrument such as shown in FIG. 2.

FIG. 6 shows LIBS analysis results of cheese using a portable instrument such as shown in FIG. 1.

FIG. 7 shows LIBS analysis results of coffee beans using a benchtop instrument such as shown in FIG. 2.

FIG. 8 shows LIBS analysis results of coffee beans using a portable instrument such as shown in FIG. 1.

FIG. 9 shows LIBS analysis results of balsamic vinegar using a benchtop instrument such as shown in FIG. 2.

FIG. 10 shows LIBS analysis results of balsamic vinegar using a portable instrument such as shown in FIG. 1.

FIG. 11 shows LIBS analysis results of vanilla extract using a benchtop instrument such as shown in FIG. 2.

FIG. 12 shows LIBS analysis results of vanilla extract using a portable instrument such as shown in FIG. 1.

FIG. 13 shows LIBS analysis results of spice using a benchtop instrument such as shown in FIG. 2.

FIG. 14 shows LIBS analysis results of spice using a portable instrument such as shown in FIG. 1.

FIG. 15 shows a summary of LIBS analysis results for classifying different food products using both portable and benchtop LIBS devices identifying the best classification methods from among the various machine learning methods shown in FIG. 4.

FIG. 16 shows a summary of LIBS analysis results for classifying different food products using both portable and benchtop LIBS devices identifying the dominant peaks for classification.

FIG. 17 shows raw LIBS spectra for various food analysis shown in FIGS. 5-16 using the benchtop device.

FIG. 18 shows raw LIBS spectra for various food analysis shown in FIGS. 5-16 using the portable device.

FIG. 19 shows exemplary methods of linking a lanthanide to an antibody.

FIG. 20 shows exemplary methods for creating a LIBS assay.

FIG. 21 shows results for detecting various lanthanides.

FIG. 22 shows a diagram for various cytokine detection methods according to certain embodiments.

FIG. 23 shows an exemplary benchtop LIBS system useful in molecular detection.

FIG. 24 shows exemplary lateral flow immunoassay (LFIA) techniques using gold nanoparticles.

FIG. 25 shows exemplary antibody label options for biomolecular labeling.

FIG. 26 shows results for characterization and selection of gold nanoparticles for assays according to certain embodiments.

FIG. 27 shows exemplary paper geometry for molecular detection LFIA methods according to certain embodiments.

FIGS. 28A and 28B show an exemplary LFIA-LIBS assay for detecting IL-6 labeled with Eu according to certain embodiments both before application of sample (FIG. 28A) and after application of the sample (FIG. 28B).

FIG. 29 shows LIBS detection of the Eu bound IL-6 on the strip shown in FIGS. 28A and 28B.

FIG. 30 shows results for the detection of IL-6 and IP-10 using LFIA-LIBS techniques according to certain embodiments.

FIG. 31 shows the quantification of IL-6 using LFIA-LIBS methods according to certain embodiments.

FIG. 32 lists additional cytokines that may be detected using molecular detection methods described herein.

FIG. 33 shows additional molecules detectable using methods of the invention.

FIG. 34 shows doping on Nitrocellulose paper (square dimension: 6*6 mm²). Elements dissolved in nitric acid and dried onto paper.

FIG. 35 is a schematic of new bench-top instrument.

FIG. 36 shows spectrometer performance comparison.

FIG. 37 shows a Raman test and associated results.

FIG. 38 shows Elastic net selection.

FIG. 39 shows a diagram of data processing.

FIG. 40 shows a diagram of algorithm process.

FIG. 41 shows an averaged spectrum of vanilla.

FIG. 42 shows a peak analysis of vanilla.

FIG. 43 shows an averaged spectrum of vanilla.

FIG. 44 shows an averaged spectrum of vinegar.

FIG. 45 shows an averaged spectrum of vinegar.

FIG. 46 shows an averaged spectrum of coffee.

FIG. 47 shows an averaged spectrum of coffee in other systems.

FIG. 48 shows an averaged spectrum of coffee.

FIG. 49 shows an averaged spectrum of cheese (from 1st Bench-top).

FIG. 50 shows an averaged spectrum of cheese (from 1st Bench-top).

FIG. 51 shows a calibration Na peak (from 1st Bench-top Cheese data).

FIG. 52 shows an averaged spectrum of cheese (from Hand-held).

FIG. 53 shows an averaged spectrum of spices.

FIG. 54 shows an averaged spectrum of spices.

FIG. 55 shows an averaged spectrum of olive oils.

FIG. 56 shows cheese from bench-top instrument.

FIG. 57 shows cheese from bench-top instrument.

### DETAILED DESCRIPTION

The invention generally relates to the application of spectroscopic techniques such as laser-induced breakdown spectroscopy (LIBS) for sample classification and verification (e.g., food authentication or fingerprinting) as well as rapid molecular detection (e.g., cytokine detection to profile immune response and diagnose cytokine storms).

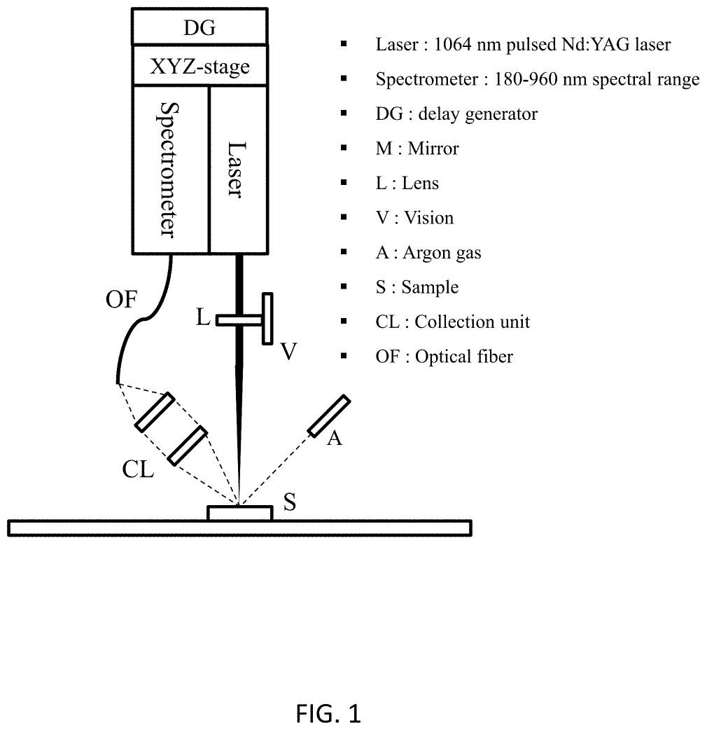

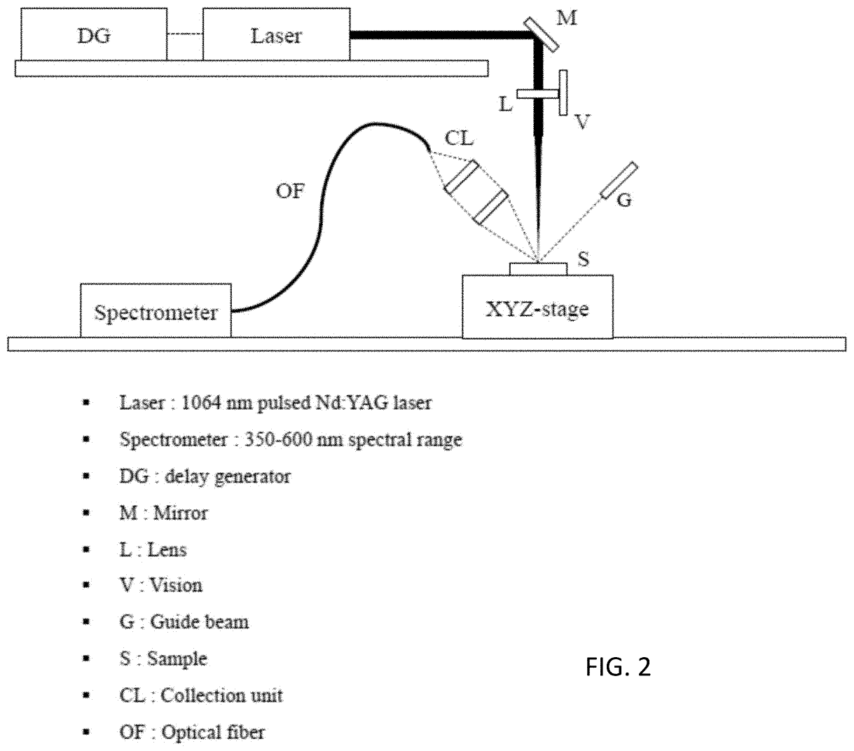

LIBS devices such as those shown in FIG. 1 (portable) and FIG. 2 (benchtop) can be used with systems and methods of the invention to generate atomic emission spectra. In various embodiments, other spectroscopy techniques including Raman spectroscopy or FTIR spectroscopy may be used to generate emission spectra of a sample. As discussed below LIBS can be advantageous for systems and methods of the invention due to its speed of operation, its ability to analyze very small volumes of sample and the ability to work with both solid and liquid samples.

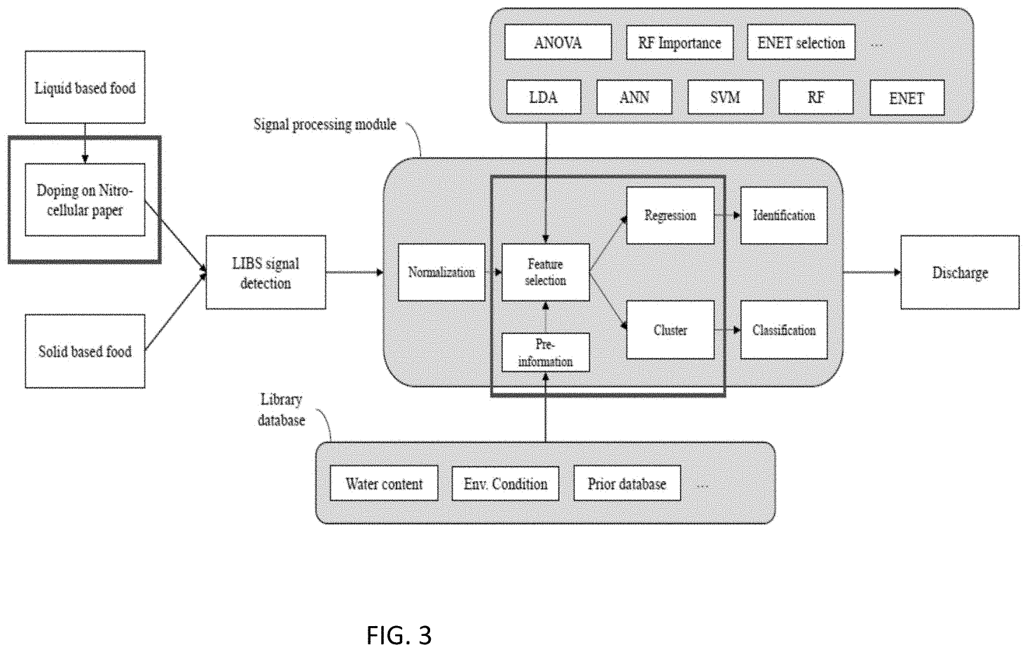

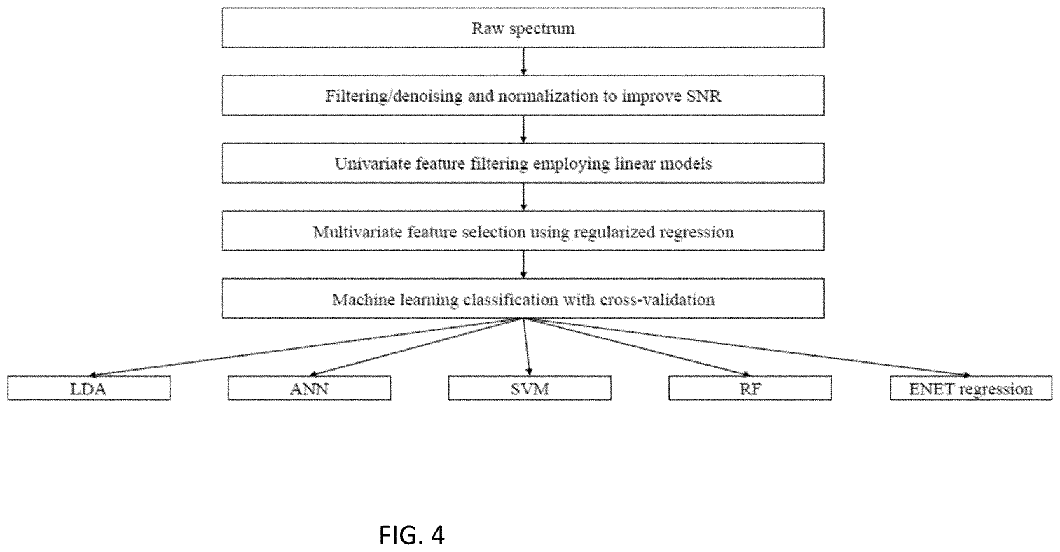

In certain embodiments, systems and methods of the invention can be used for sample classification and authentication including food samples. An exemplary process is diagrammed in FIG. 3. A sample is obtained (in this case a food sample). Liquid samples can be doped onto nitrocellulose paper while solid foods can be directly analyzed. LIBS or other spectroscopic techniques can then be used to generate an emission spectrum of the sample. The signal processing module can then normalize the sample followed by automated feature selection. Additional inputs including pre-information from a library database can be added and various machine learning techniques can be used in the feature selection process. Regression and cluster analyses can be used for identification and classification. Exemplary data processing and spectral analysis steps are diagramed in FIG. 3. A raw spectrum (e.g., obtained via LIBS) can be filtered and denoised and normalized to improve the signal-to-noise ratio. Univariate feature filtering can then be performed employing linear models. Multivariate feature selection can be performed using regularized regression. Machine learning classification with cross-validation can then be performed using one or more techniques such as linear discriminant analysis (LDA), an artificial neural network (ANN), support vector machine (SVM), random forest (RF), or elastic net (ENET) regression.

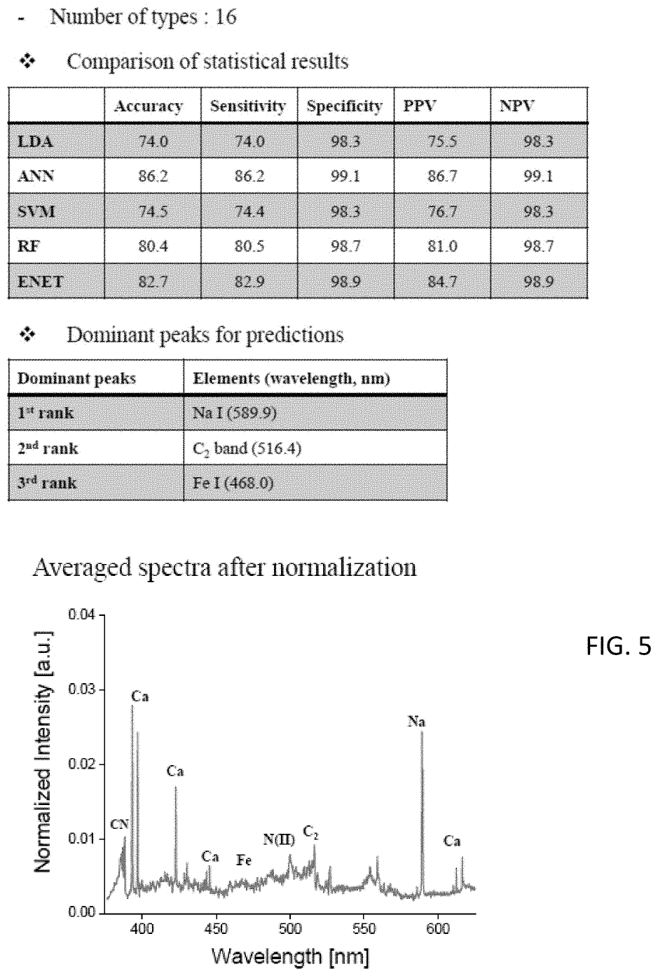

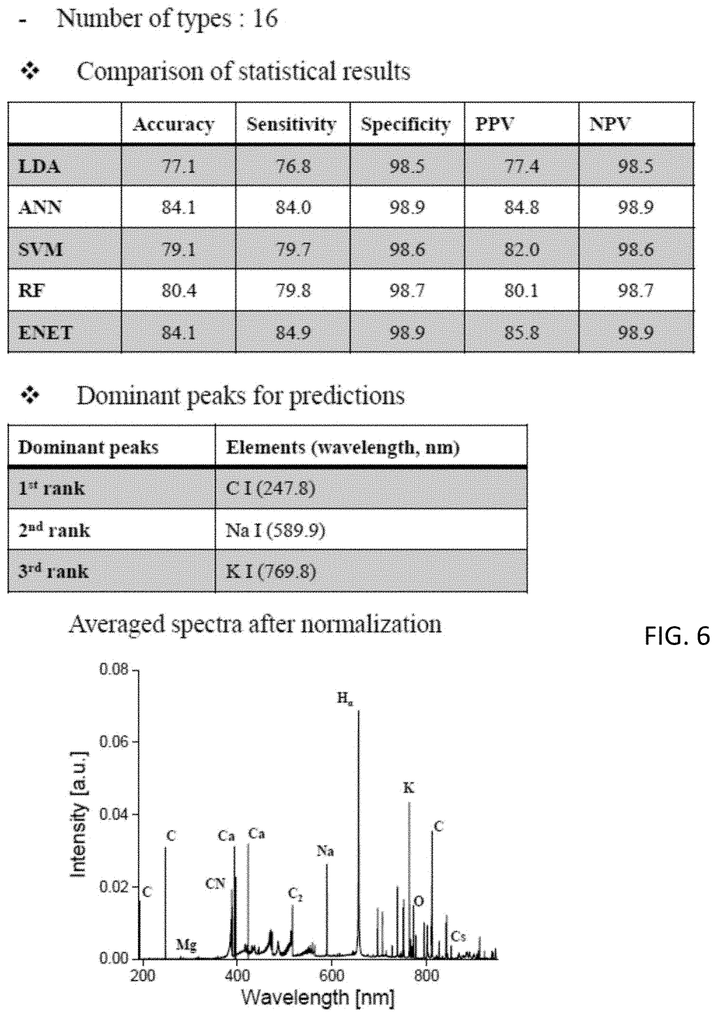

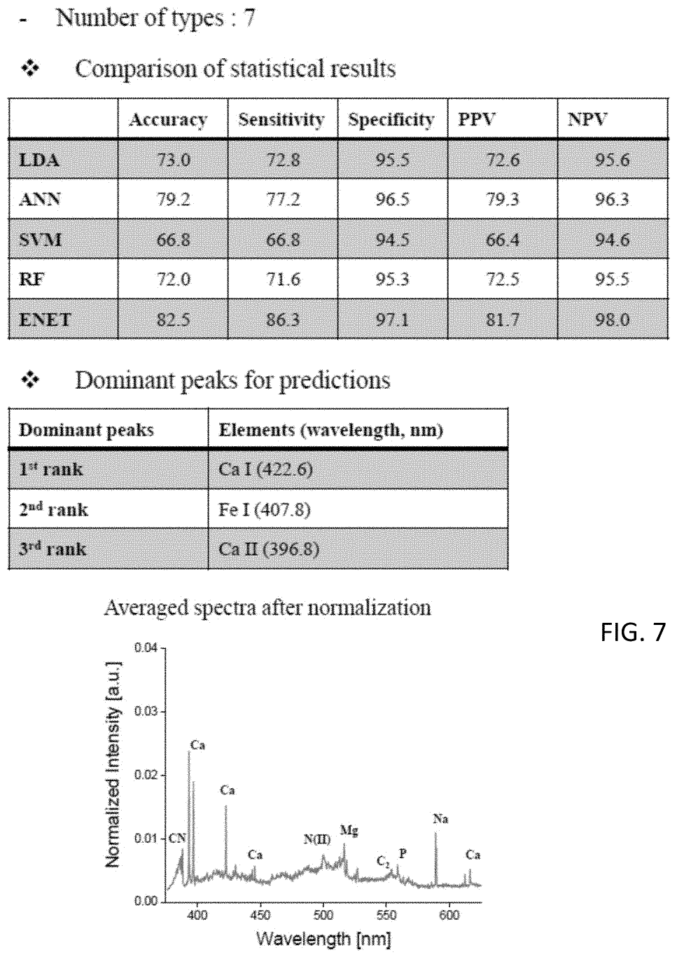

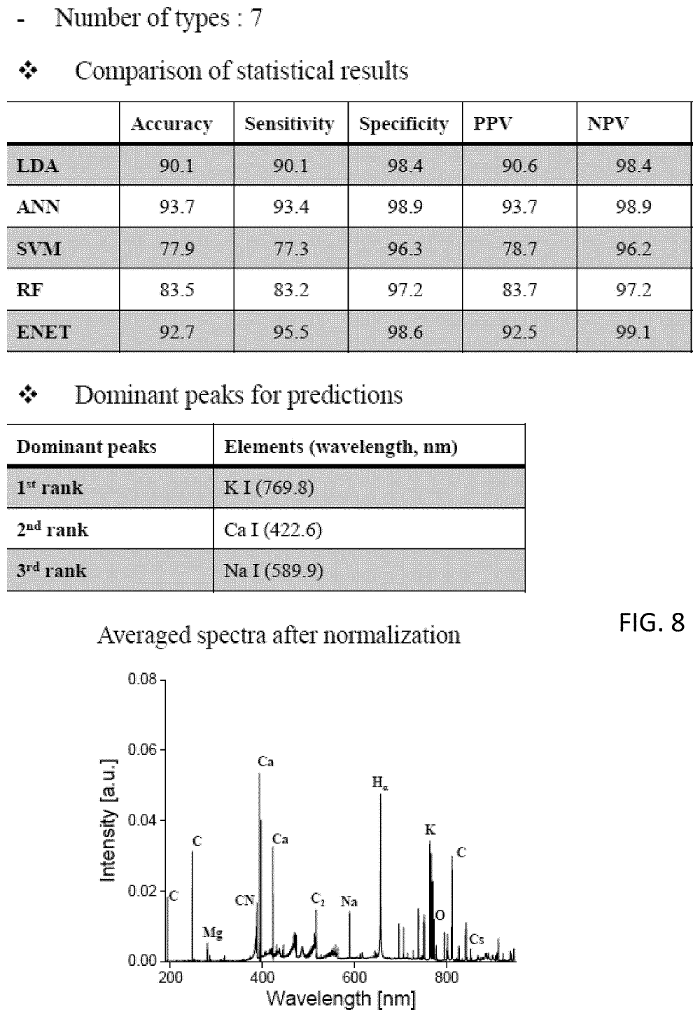

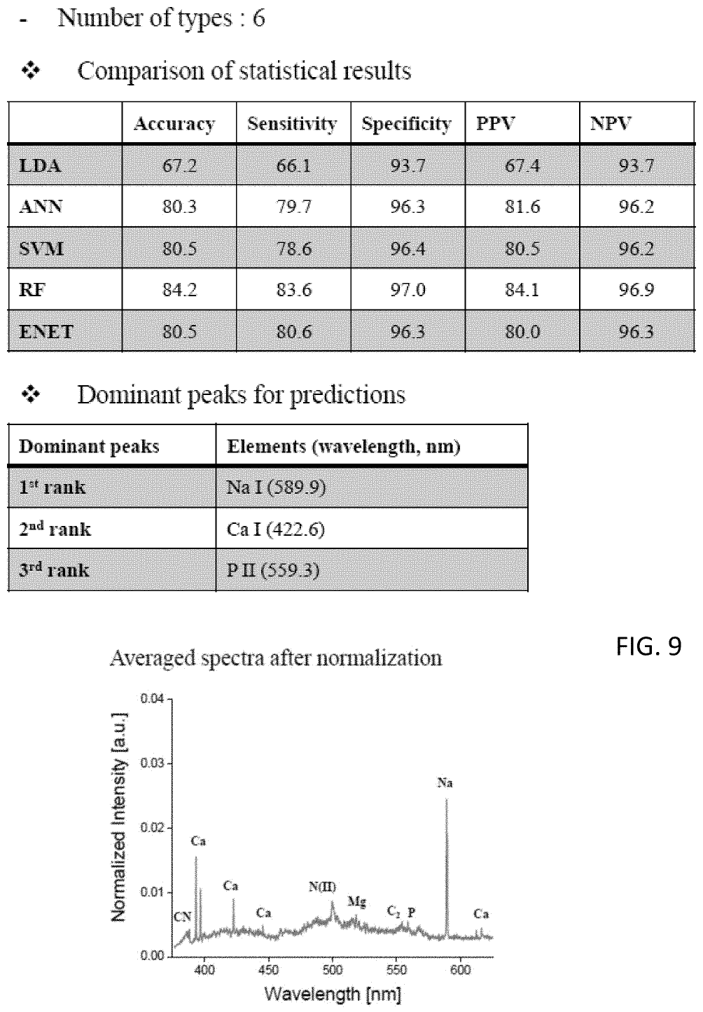

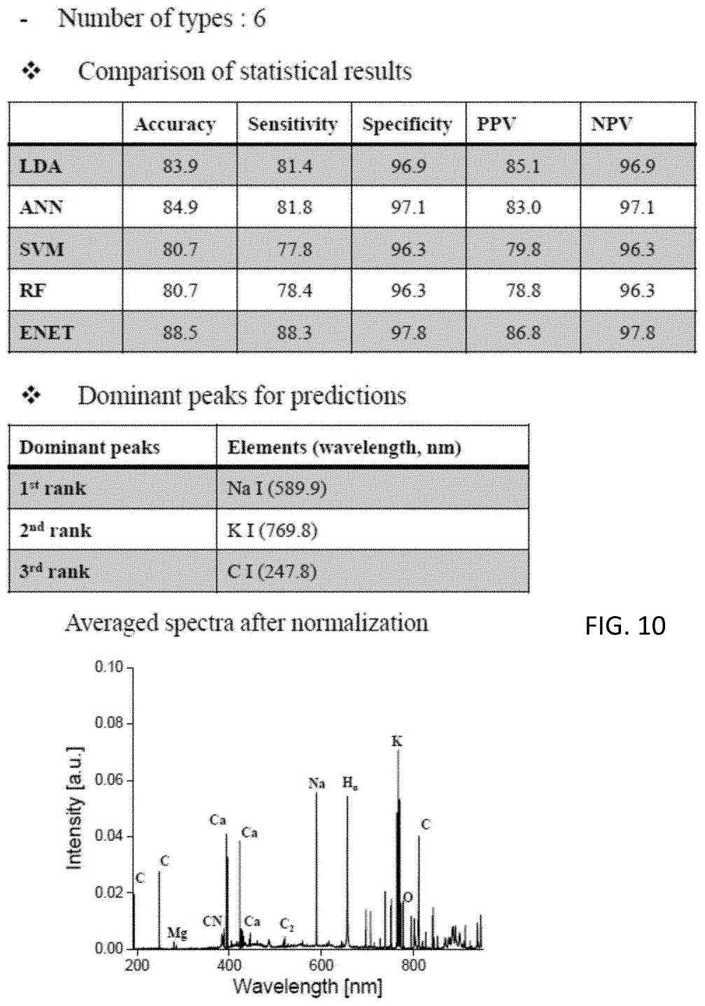

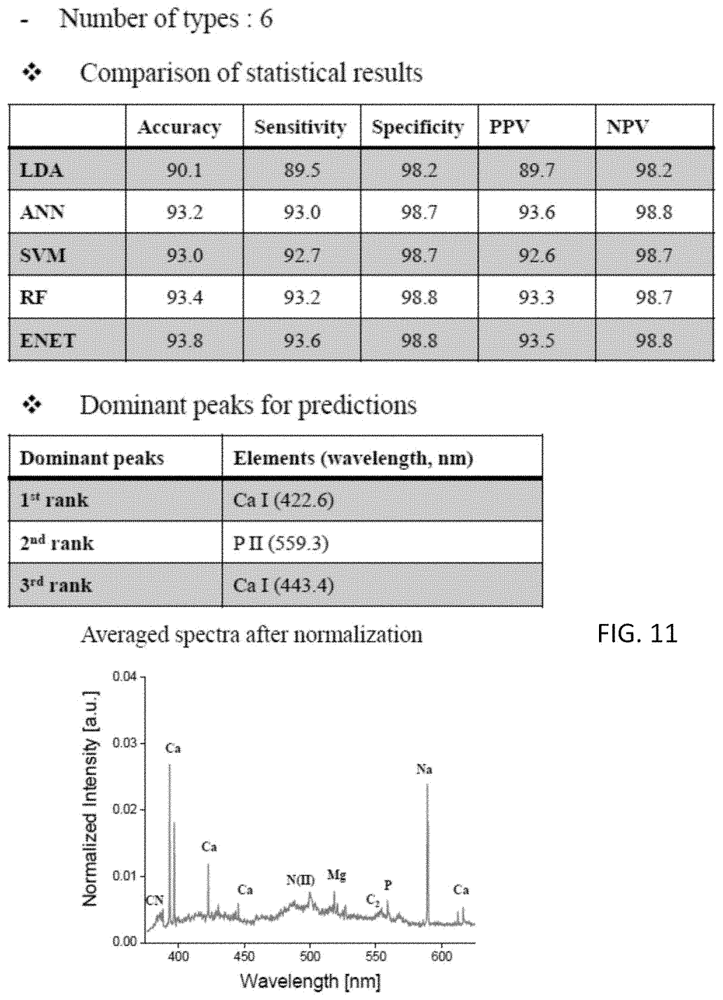

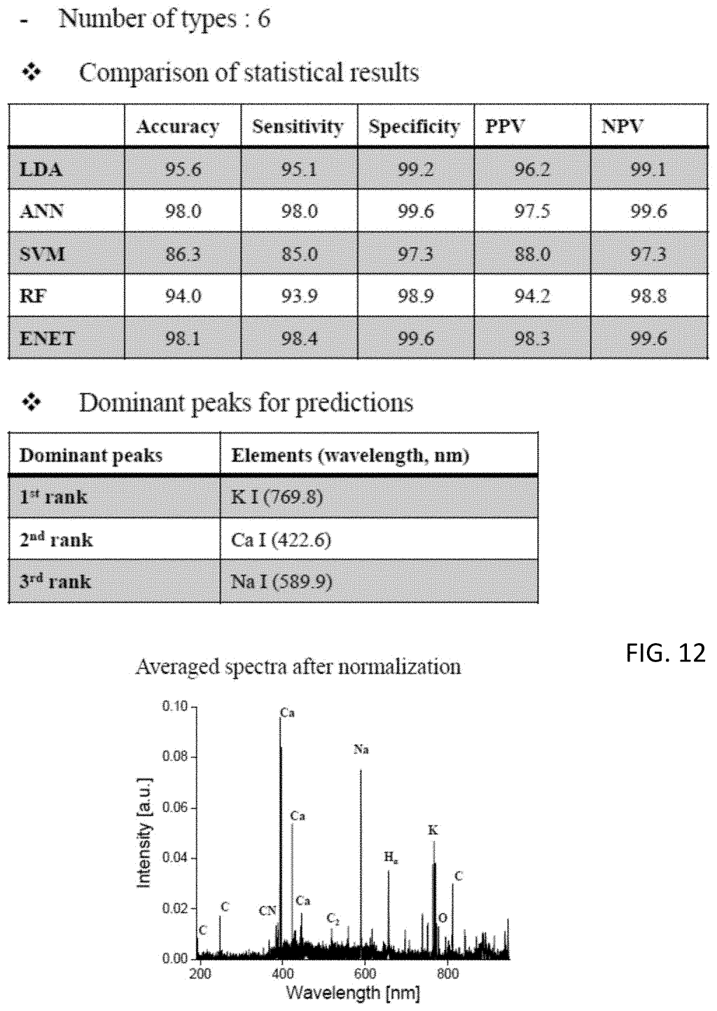

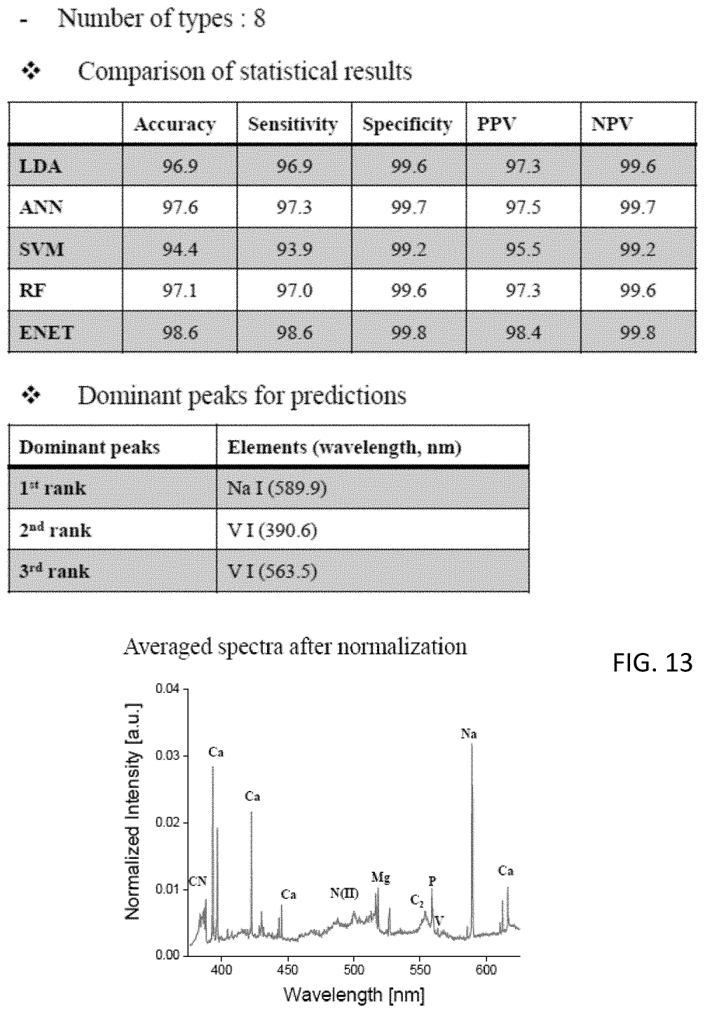

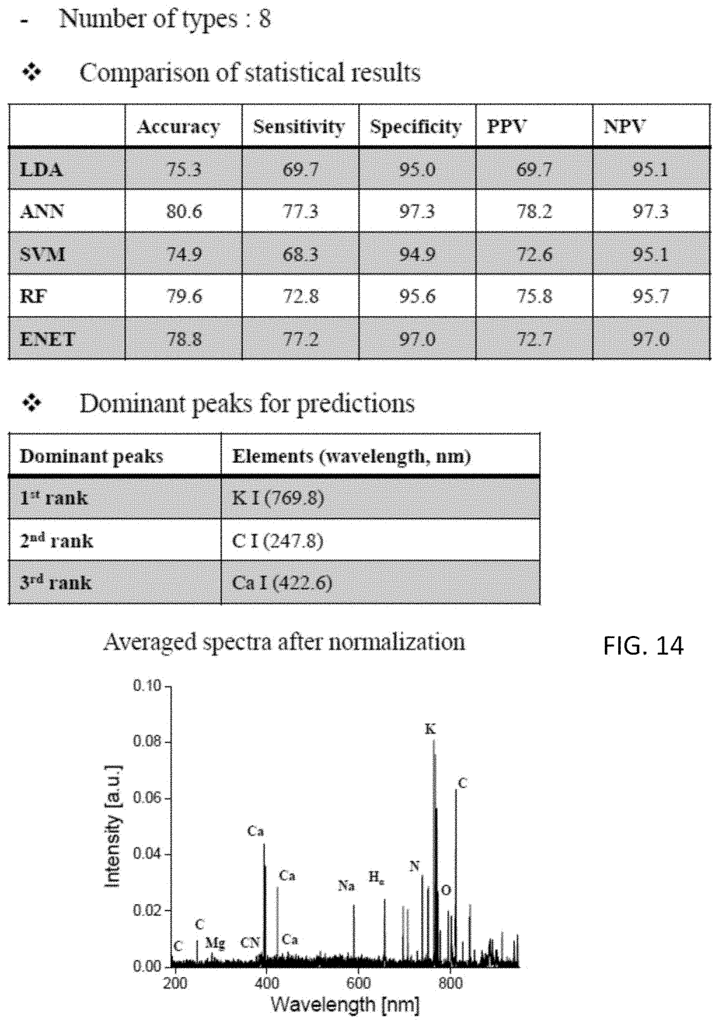

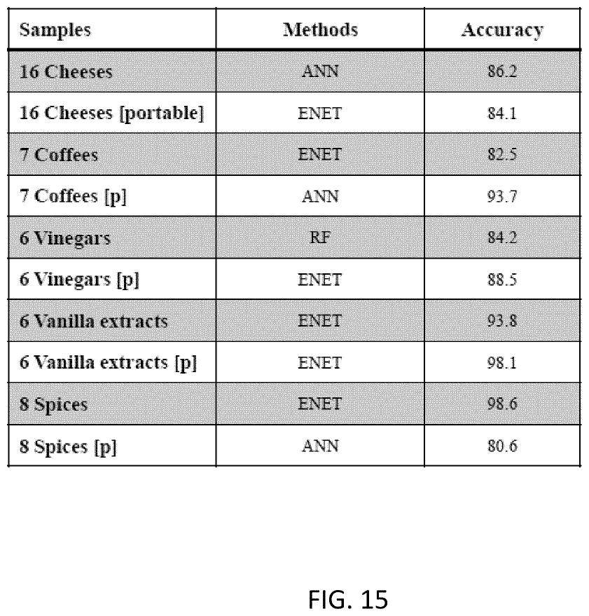

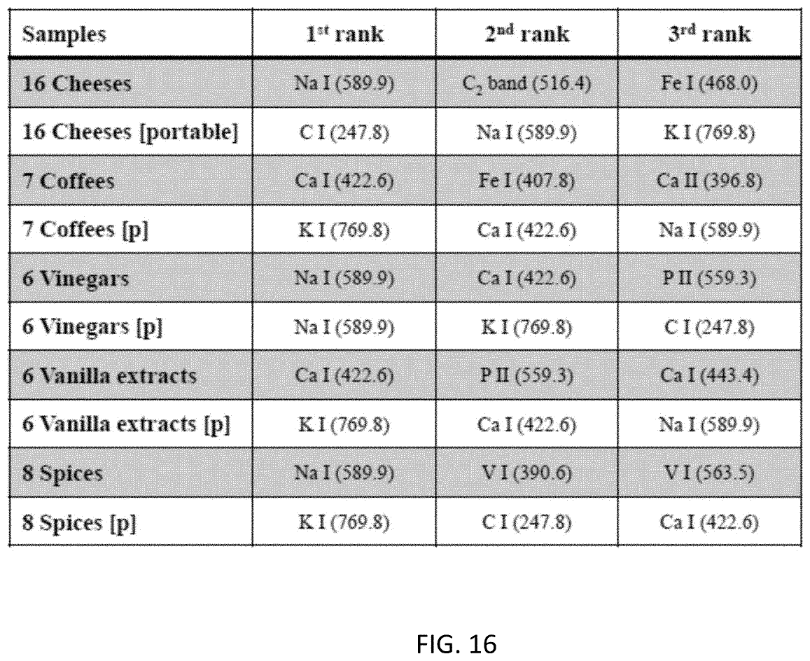

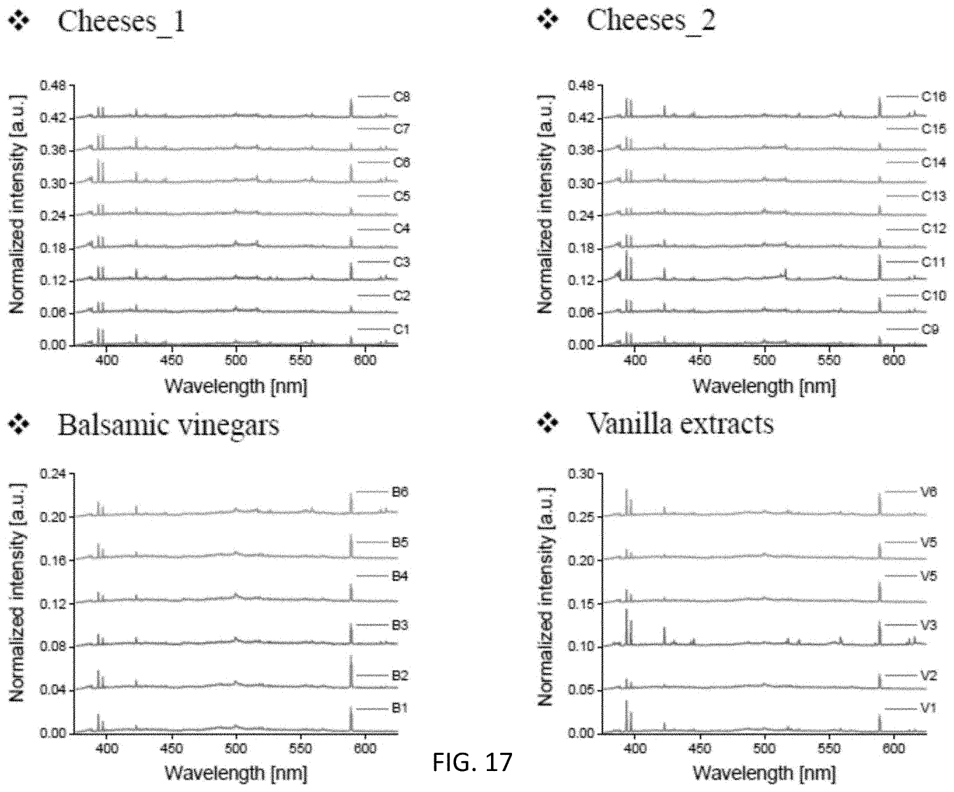

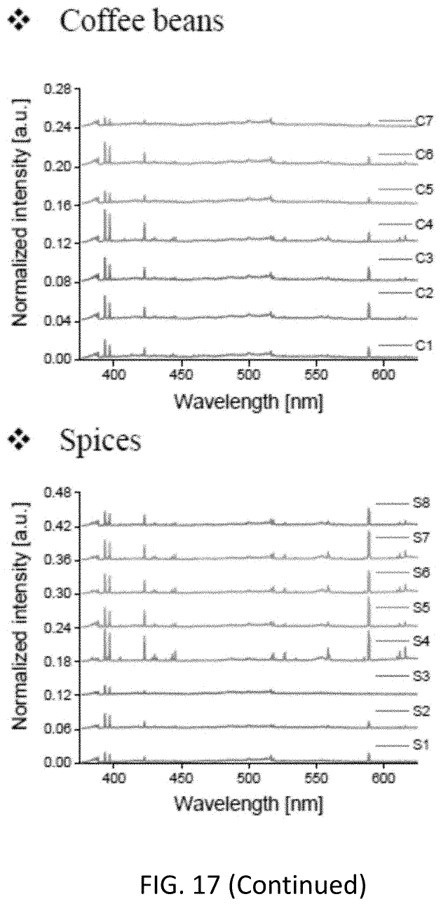

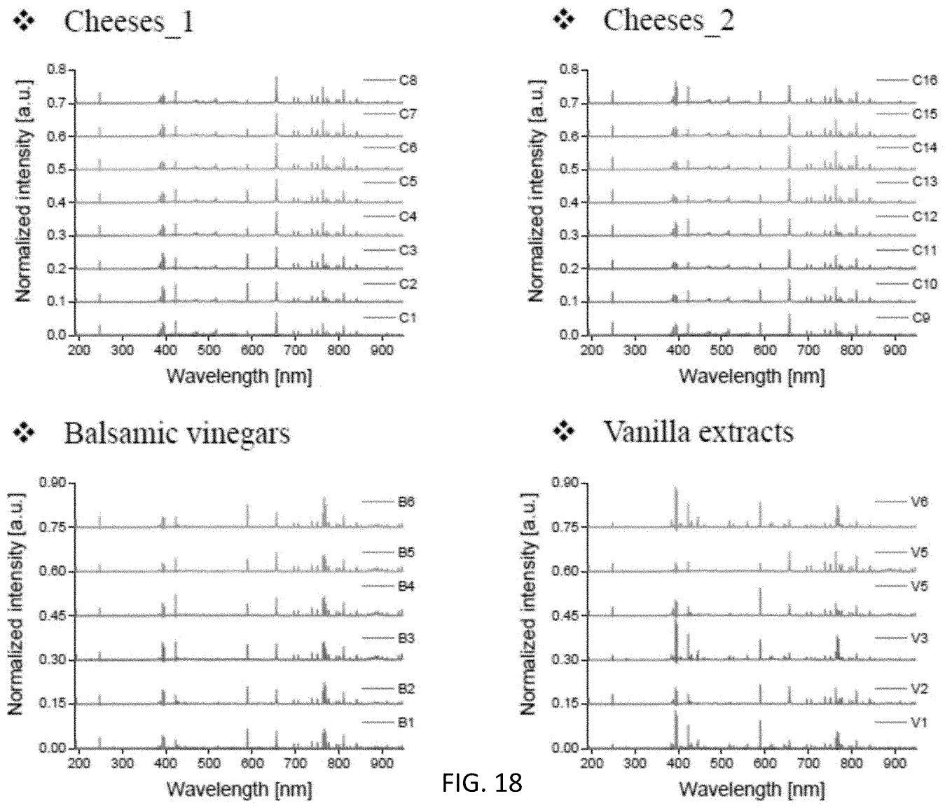

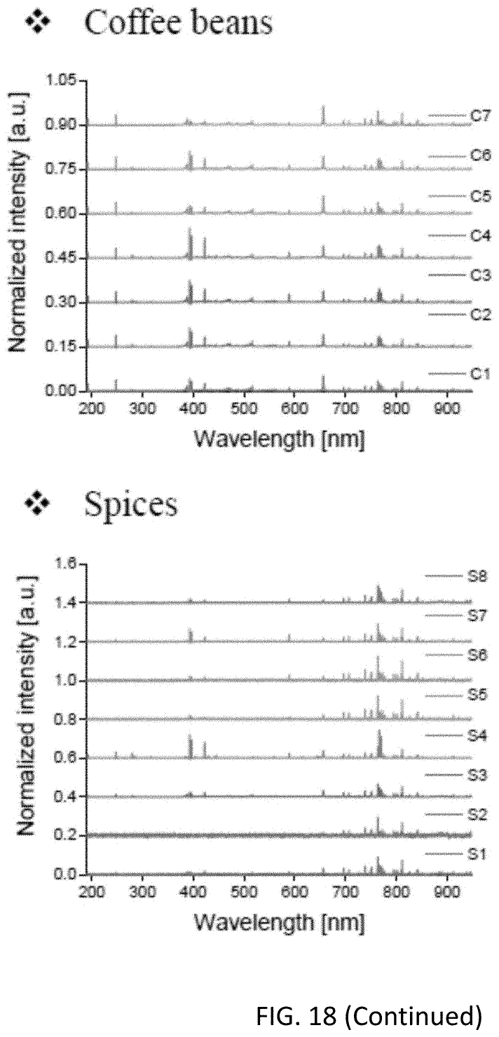

Exemplary analyses are shown for various food types in FIGS. 5-14 using either a bench-top LIBS device as shown in FIG. 2 or a portable device as shown in FIG. 1. Statistical results for each are compared for each of 5 different machine learning techniques (LDA, ANN, SVM, RF, and ENET). Dominant peaks for predictions and the averaged spectra after normalization are also shown. Results are shown for 16 types of cheese (FIG. 5—bench-top and FIG. 6—portable), 7 types of coffee beans (FIG. 7—bench-top and FIG. 8—portable), 6 types of balsamic vinegar (FIG. 9—bench-top and FIG. 10—portable), 6 types of vanilla extract (FIG. 11—bench-top and FIG. 12—portable), 8 types of spices (FIG. 13—bench-top and FIG. 14—portable). Overall results are shown in FIGS. 15 and 16 with FIG. 15 showing the best classification method for each food product with either bench-top or portable device and FIG. 16 showing the dominant peaks for classification. The raw LIBS spectra are shown in FIG. 17 (bench-top analyzer) and FIG. 18 (portable analyzer).

Additional discussion of machine learning classification of spectra for food authentication using LIBS-generated atomic spectra is found in Example 1 and the Appendix.

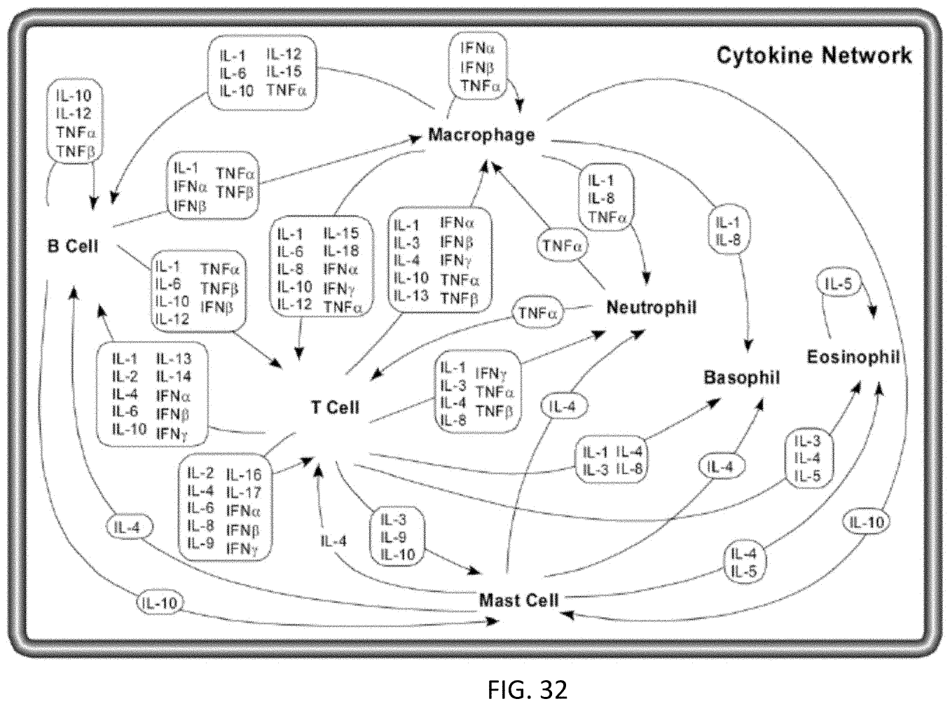

In certain embodiments, lateral flow immunoassays (LFIA) can be used in conjunction with LIBS analysis to detect and quantify molecules in a sample including cytokines in biological samples such as biological fluids from a patient to assess potential for cytokine storms associated with severe cases of COVID-19. Such methods may be used to detect any number of cytokines. FIG. 32 shows exemplary cytokines that have been identified in various biological states. In various embodiments, any of the disclosed cytokines may be detected and or quantified using the methods described herein alone or in combination (multiplex analysis) simply by adding target capture molecules such as antibodies specific to the target cytokine. In various embodiments cytokine profiles may be determined that may be indicative of a biological state and may be investigated as a panel using LFIA-LIBS methods herein.

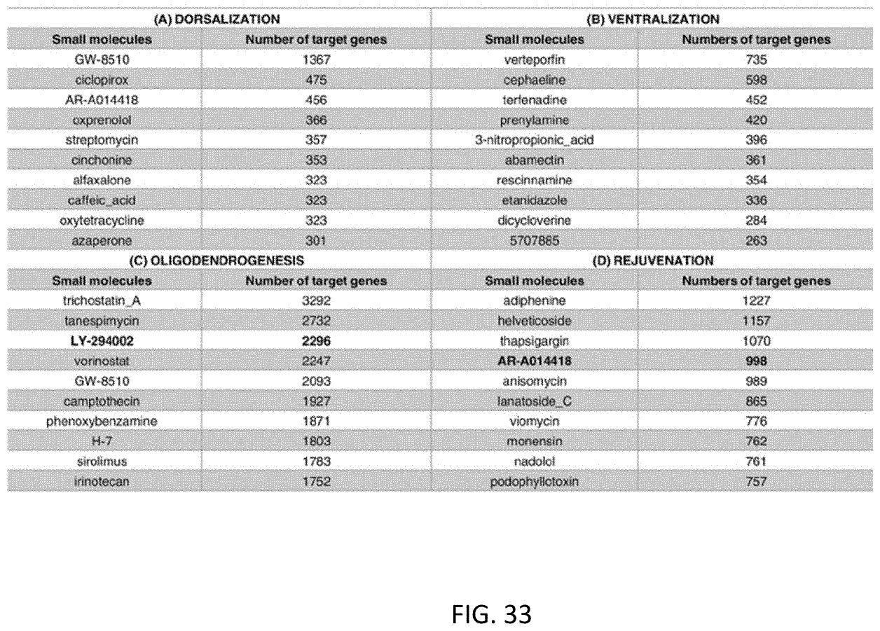

As discussed above, the molecule detection methods described herein can be applied to any molecule capable of being specifically bound by a metal (or other elemental tag) conjugated capture moiety (e.g., an antibody). Additional potential targets are listed in FIG. 33 along with various other drugs, proteins, enzymes, hormones, polypeptides, or nucleic acid.

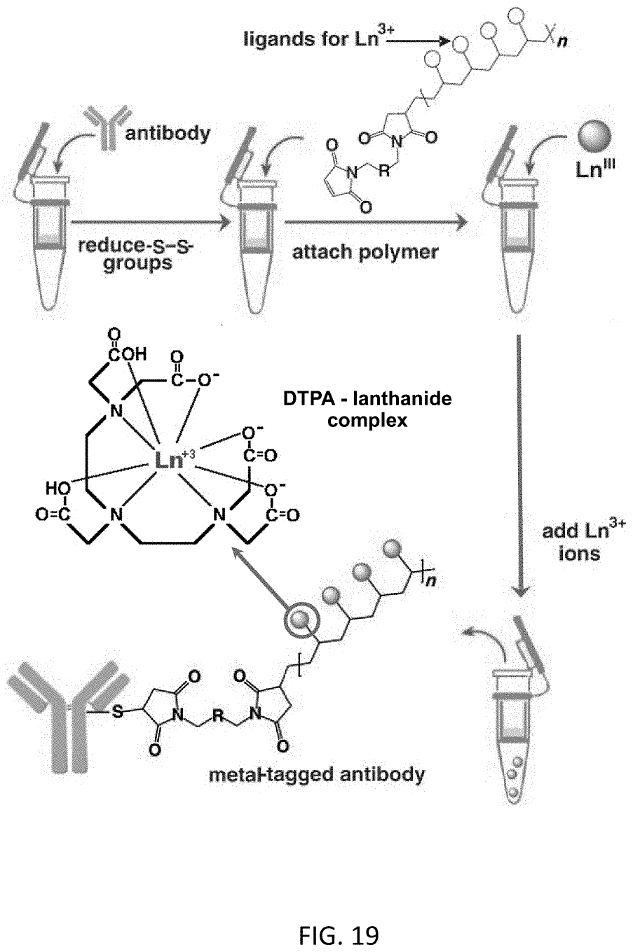

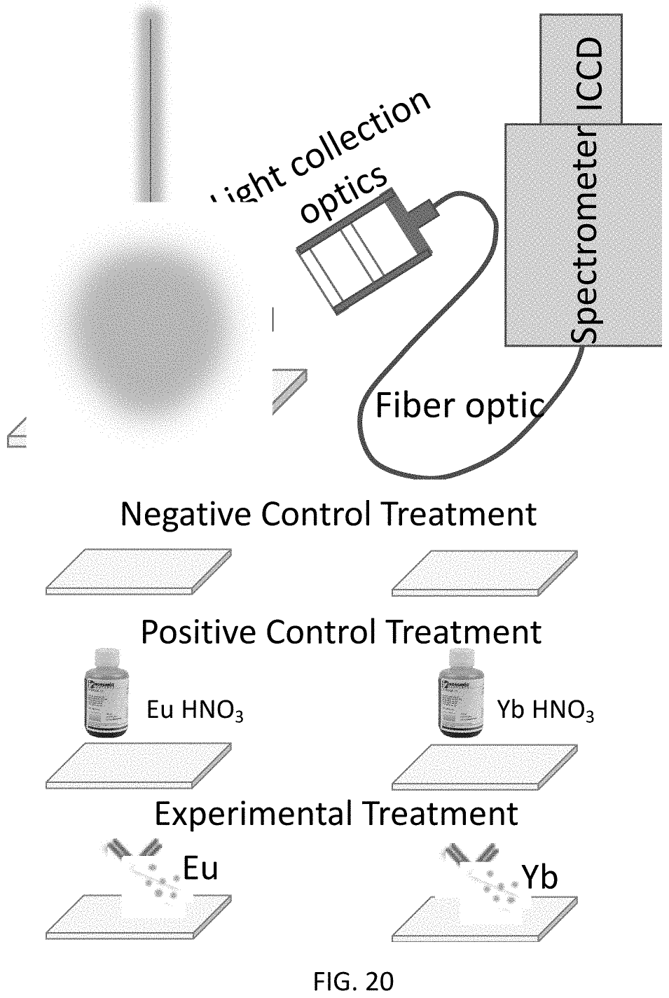

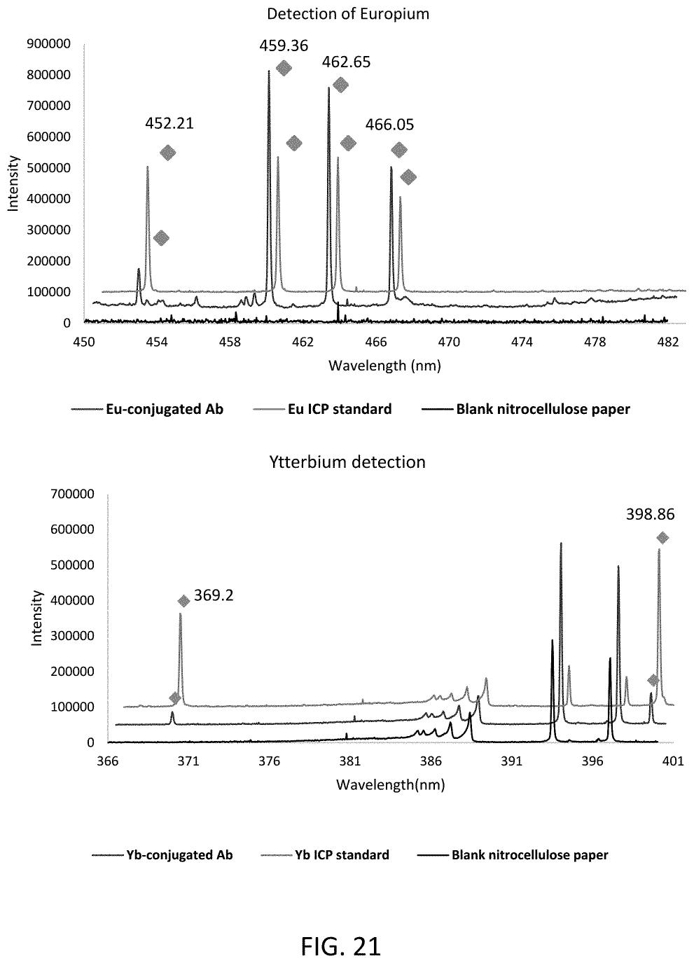

As discussed below, metal labels can be attached to antibodies as tags. In certain embodiments, lanthanides may be used as tags and can be attached to a target-specific antibody as shown in FIG. 19. Lanthanides can provide the good performance in COVID assay using LIBS. The initial step is to link the lanthanides to antibody. The antibody of interest is subjected to selective reduction of -SS-groups (disulfide) to produce reactive -SH thiol groups, which are reacted with the terminal maleimide groups of a polymer bearing metal-chelating ligands along its backbone. Subsequently, the polymer-bearing antibodies are purified, treated with a given lanthanide ion, and then purified again. With the same chemical reaction, antibody can be labeled with any lanthanides. Three treatments were compared by LIBS to confirm successful conjugation of metal ions, polymers and antibodies as shown in FIG. 20. The experimental treatment is metal-complexed antibodies on a piece of paper along with a positive control consisting of metal dissolved in nitric acid loaded onto nitrocellulose paper, and the negative control of a blank piece of nitrocellulose paper. Here Eu- and Yb- conjugated antibodies were analyzed. Results, as shown in FIG. 21, indicated that the positive control produced the most intense signal, followed by the characteristic peaks in the experimental samples, indicating the successful conjugation of lanthanides to polymers to antibodies.

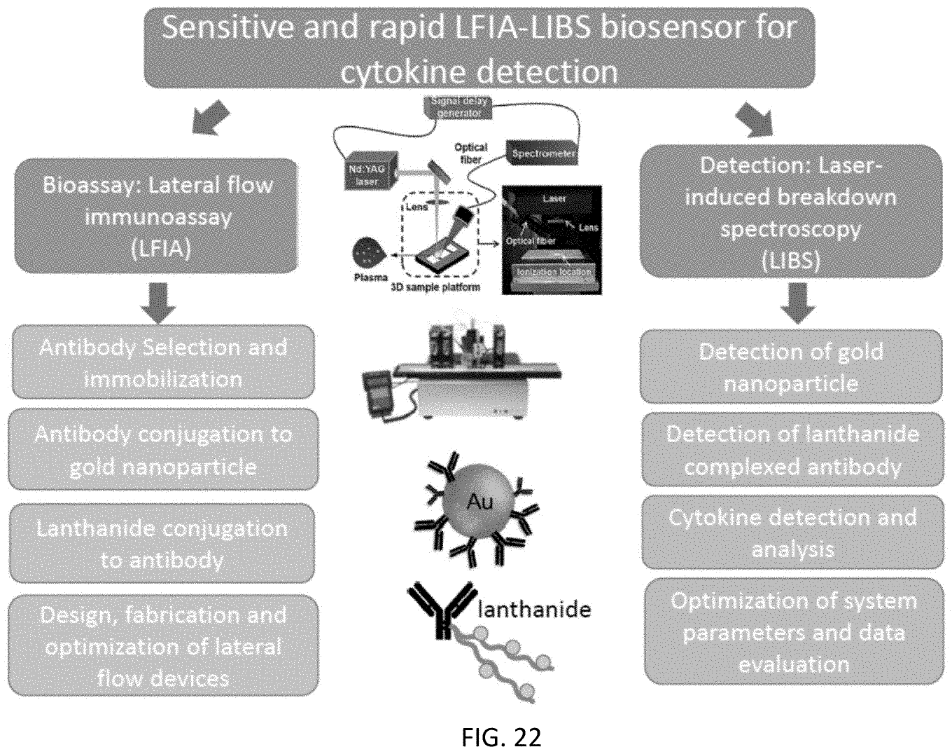

FIG. 22 diagrams a design approach to rapidly detect cytokines. It combines detection of molecules based on laser-induced breakdown spectroscopy with a lateral flow immunoassay (LFIA-LIBS) to deliver a quantitative clinical analysis platform. Two features of this new technology are the incorporation of paper-based assays and detection of lanthanides labels by LIBS to quantitate cytokine levels. Both features benefit from characterization and optimization to simultaneously achieve the desired results.

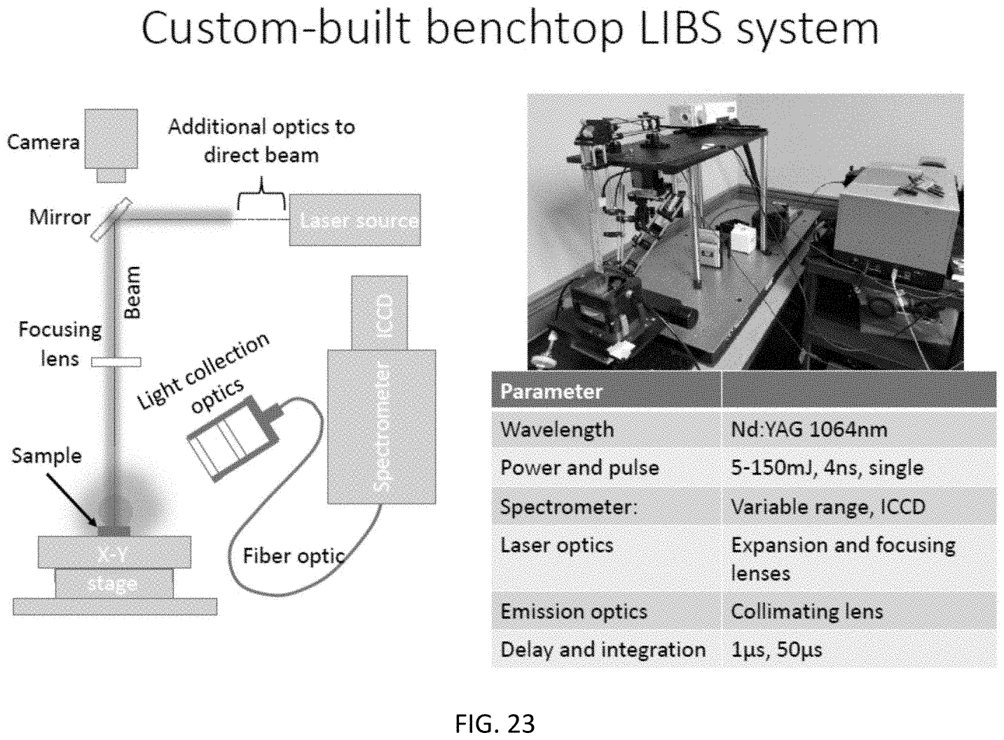

As discussed below, in using LIBS, when a material is hit with a high energy, pulsed laser beam. The material ablates and light is produced. The spectral analysis of that light can tell what elements compose the sample. FIG. 23 shows a custom-built LIBS instrument the parameters used in the detection platform.

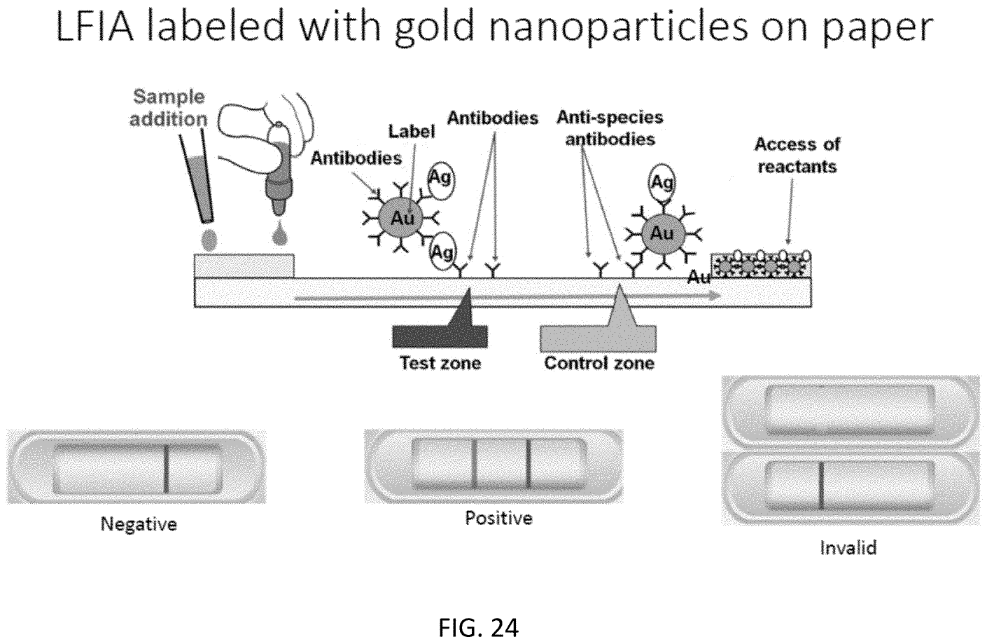

FIG. 24 shows an exemplary LFIA assay. The most common modality that's commercially available is visual detection of gold nanoparticles in a strip of nitrocellulose paper, which is a gold standard of LFIA. Antibodies targeting the analytes are added to a specific region of the nitrocellulose paper, called the test line. As the sample wicks along the strip, the labeled target will bind to the test line. Since the gold nanoparticles are pink, you will see that at the test zone, a bright pink line will form if the analyte is present. Any un-bound label binds to a different region of the strip, essentially acting as the validation of the immunoassay. The tests are invalid if there is no control line present regardless of the presence of test line.

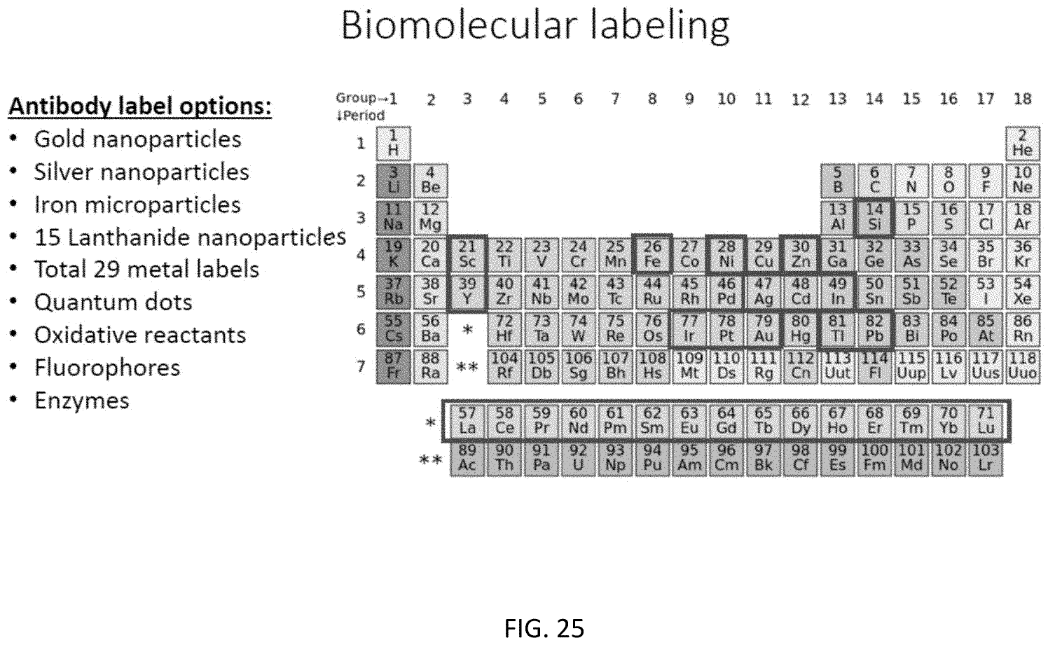

However, gold nanoparticles are not the only effective label. There are many different labels available in immunochemistry, though not all of them are used in lateral flow assays as shown in FIG. 25. Many of these labels are metals. In the broad world of antibody labeling, there are 29 metal elements available for conjugation to antibodies. A variety of detection modalities have been applied to detect metal labels in paper-based assays. Interestingly, LIBS is the only one that is capable of detecting all the metal labels.

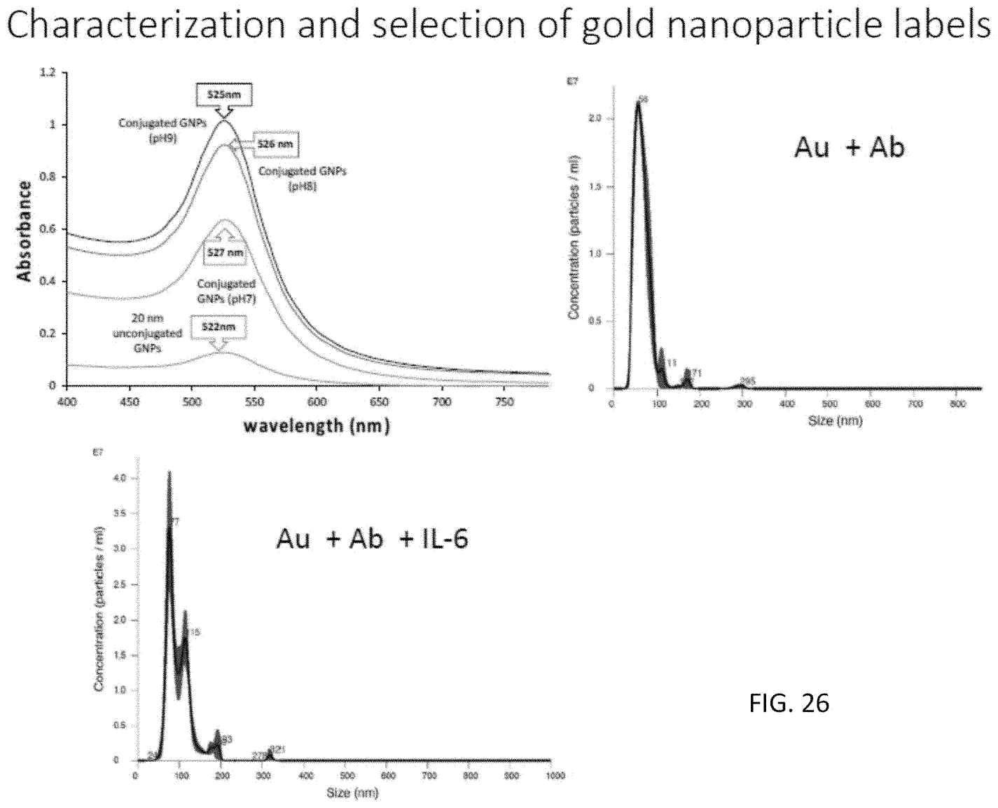

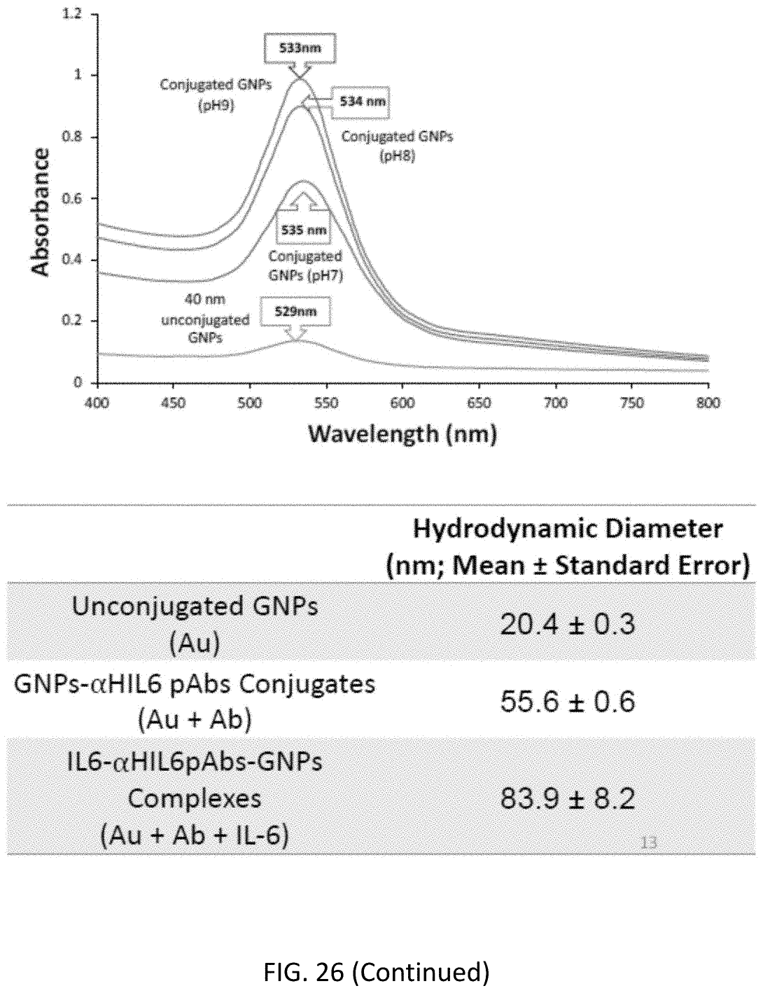

FIG. 26 illustrates the characterization and selection of gold nanoparticle labels. To prove the cytokine detection assay can work with the gold standard GNPs, the antibodies of interest were labelled with GNPs and characterized before applying them in lateral flow strips by ultraviolet-visible (UV-vis) spectroscopy and Nanoparticle Tracking Analysis. The sizes of unconjugated GNPs were 20 and 40 nm. After incubation with abs, the conjugates displayed an absorbance peak at 525 and 533 nm respectively which was red shifted from 522 and 529 nm. This illustrated the abs were successfully labeled onto the surface of GNPs. Meanwhile, the nanosight results confirmed the specific binding of the analyte IL6 to Ab on the conjugates by displaying an increased hydrodynamic size of 83.9 nm compared to 55.6 nm of the conjugates. Summarily, these characterizations suggested that the conjugates were successfully prepared and could be effectively employed for LIBS detection of IL-6.

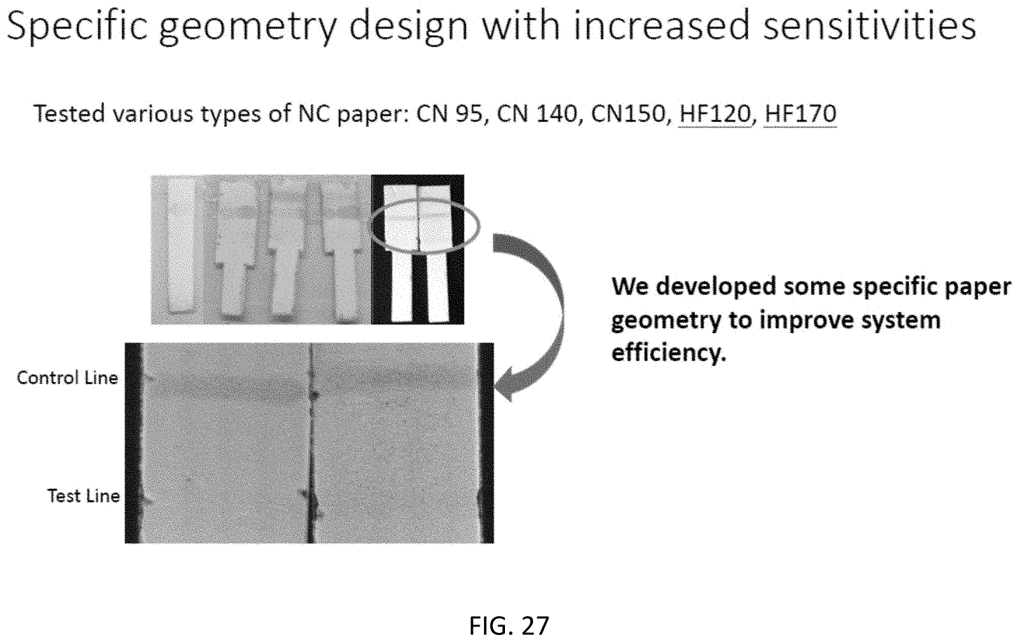

To improve the lateral flow device performance, various types of NC paper were tested with different porosity and HF120 and HF170 were found to exhibit the best fit for the design requirement of rapidity and sensitivity (results shown in FIG. 27), so they were selected for the future experiments. As follows, we investigated the influence of the geometry of nitrocellulose paper on the analytical performance. Pink positive test lines and control lines were visualized on optimized strips when samples contained IL-6 were tested.

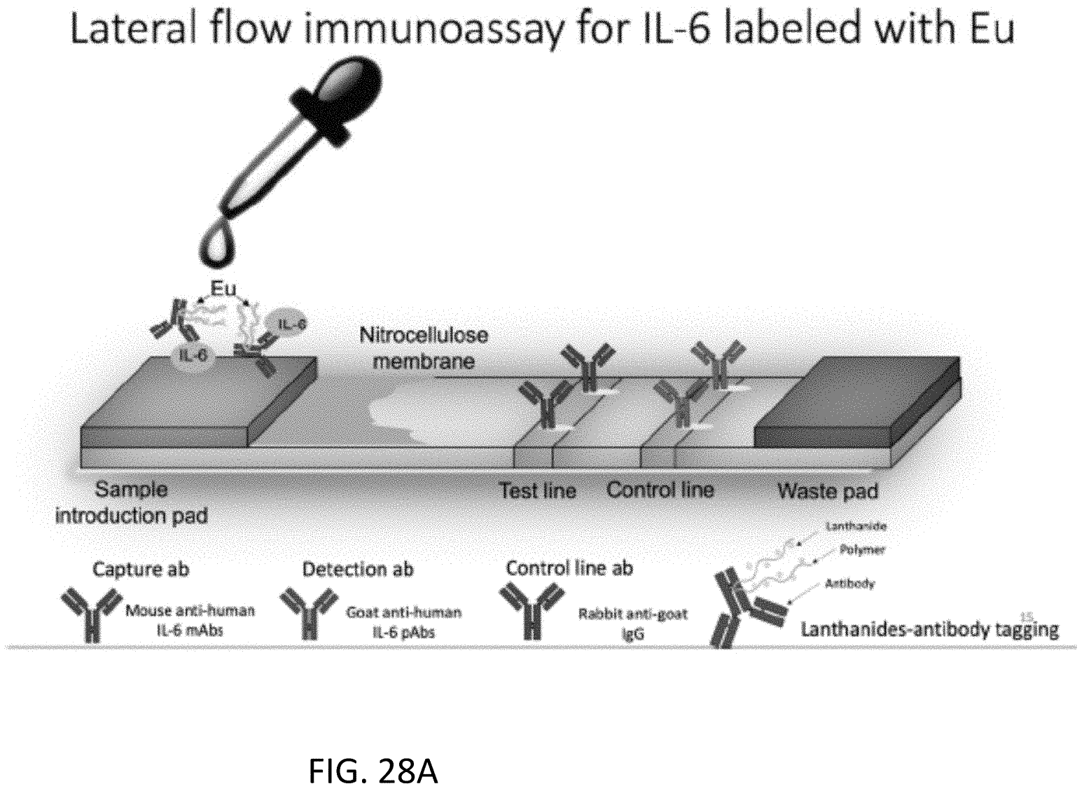

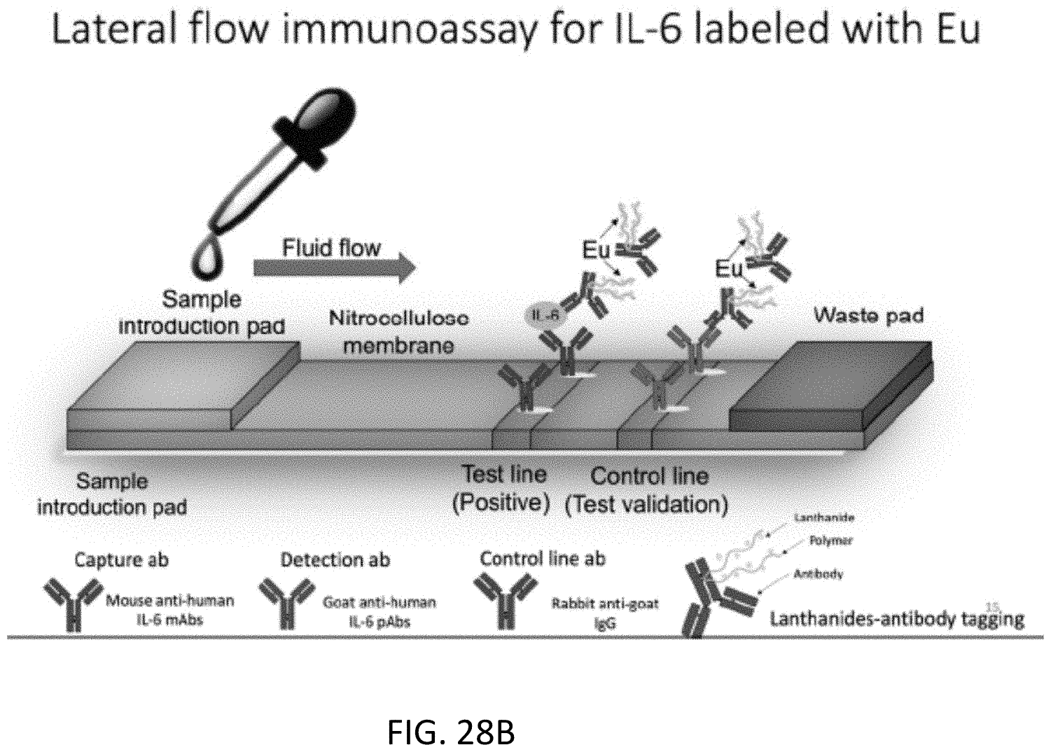

FIGS. 28A and 28B show COVID assay strips detected by LIBS before sample wicking past the test and control lines (FIG. 28A) and after (FIG. 28B). Goat anti-human IL-6 pAbs were labeled with Eu via the metal chelating polymer and introduced the mixtures into the strip, which was pretreated with different capture abs in test line and control line respectively. As the samples flow through strip, the immunoreaction between labeled detection ab, IL-6 and capture antibody, result in the accumulation of Eu on the test line of the lateral flow assay. The combination of excess Eu labeled ab and capture ab on the control (C) line ensured the validity of the lateral flow assays. The test line (T) began to resolve within 2 min and the assay was completed by 10 min.

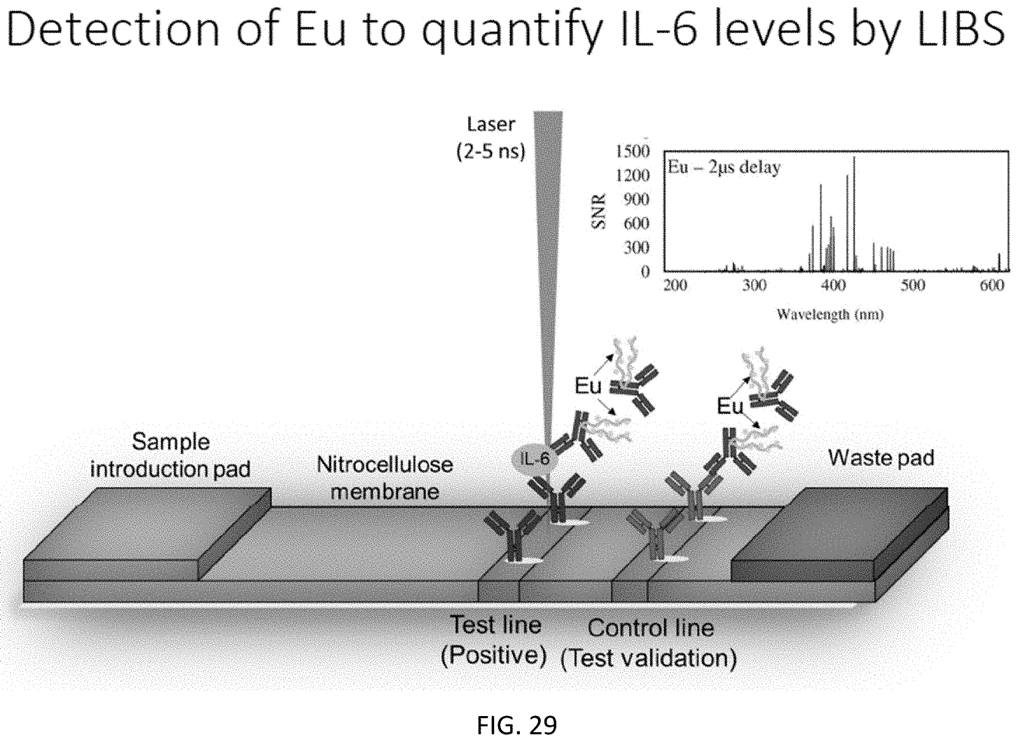

FIG. 29 shows that lateral flow test strips can be directly subjected to LIBS analysis without any pretreatment. Eu elements are ionized, and spectra were analyzed. To ensure the signal reproducibility, eight different laser spots on the T line were chosen to yield an average LIBS signal for one single test strip, and laser shots on off-line locations were used as control.

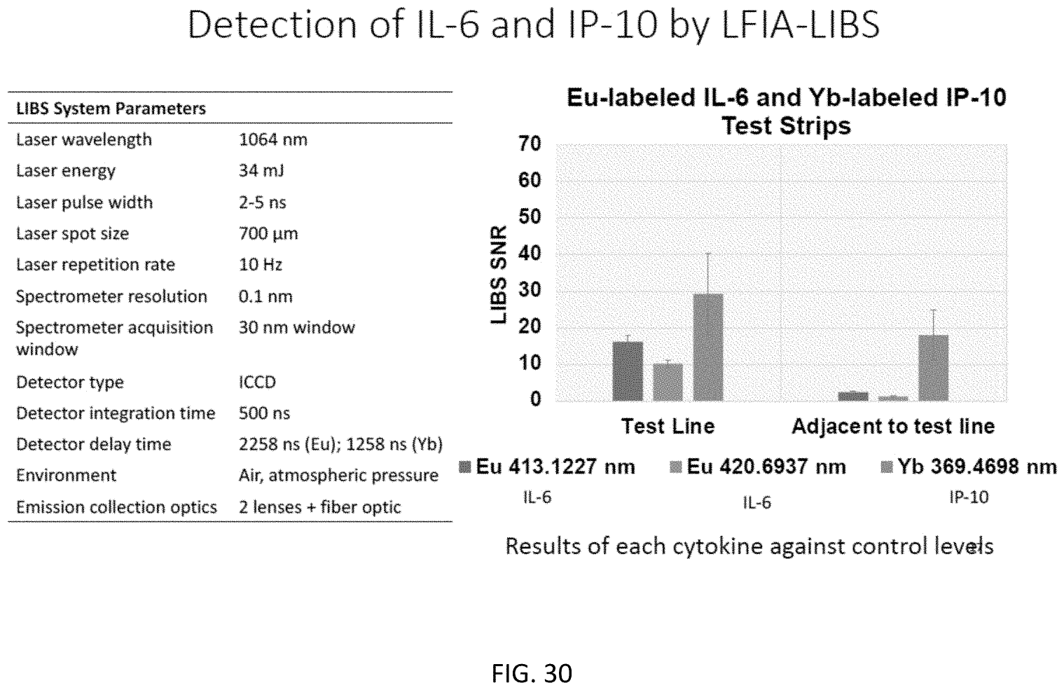

The signal intensity of Eu (II) at 420.6937 and 413.1227 nm) and Yb (II) at 369.419 nm are chosen for quantitative analysis due to its higher intensity compared to other characteristic wavelengths See FIG. 30). Eu-labeled IL-6 lateral flow assay strips and Yb-labeled IP-10 lateral flow assay strips were both successfully detected and quantified by LIBS in 15-min. This proof-of-principle biosensor will allow us to test other cytokines as well.

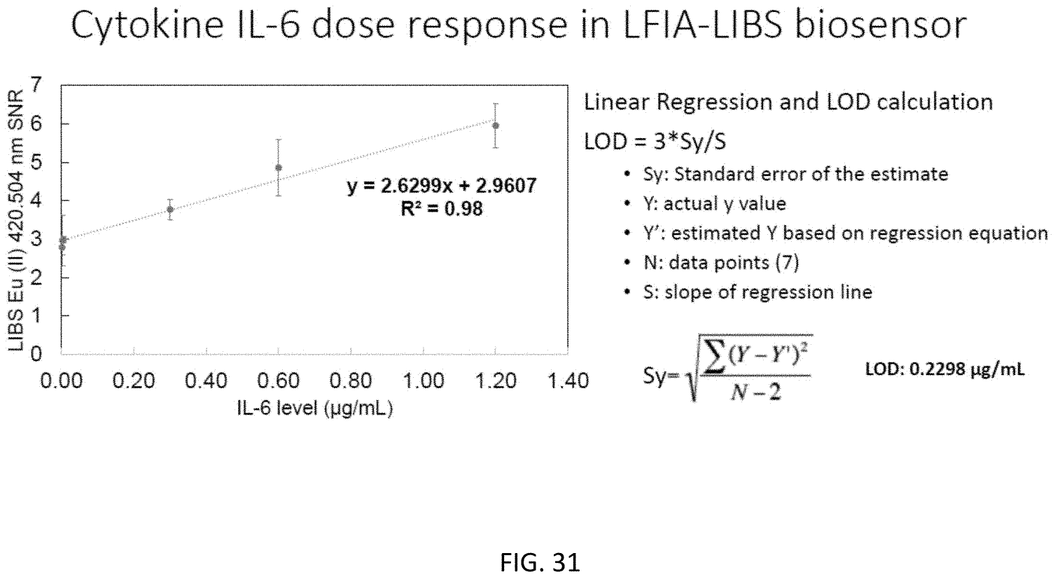

Furthermore, sensitivity of this rapid biosensor for detection of IL-6 was investigated. The Eu intensity of test line progressively strengthened with the increasing concentration of 8 points IL-6 standards from 0.01 to 1.2 μg/mL, giving a linear correlation in this range (See FIG. 31). The limit of detection (LOD) was estimated to be 0.2298 μg/mL based on the calculation shown on the right.

In various embodiments, the multiplexing capability of such biosensors as described in WO 2020/056257 may be used to detect additional cytokines or other molecules in one single test strip. Additionally, portable LIBS instruments as discussed herein with respect to food analysis can be used in molecular and cytokine profiling along with LFIA assays.

A substrate refers to a porous surface that may be composed of one or more layers. In certain embodiments, the porous surface is any cellulose-based material. An exemplary porous material is paper. In particular embodiments, the porous material is filter paper. Exemplary filter papers include cellulose filter paper, ashless filter paper, nitrocellulose paper, glass microfiber filter paper, and polyethylene paper. Filter paper having any pore size may be used. Exemplary pore sizes include Grade 1 (11 μm), Grade 2 (8 μm), Grade 595 (4-7 μm), and Grade 6 (3 μm).

In certain embodiments, the substrate is a single layer of porous material, e.g., a single layer of paper (such as nitrocellulose paper). That single layer may be functionalized with a single type of capture molecule (in multiple copies) or multiple different types of capture molecules (each type of capture molecule optionally being present in multiple copies). The substrate may also include an absorbent pad arranged beneath the single layer of porous material. In preferred embodiments for the detection of molecules such as cytokines (e.g., IL-60, the nitrocellulose paper is HF120 or HF170 available from MilliporeSigma (Burlington, Mass.).

In other embodiments, the substrate includes multiple layers of porous material, e.g., multiple layers of paper (such as nitrocellulose paper). This arrangement is an exemplary substrate for the multiplexed methods. Each layer is functionalized with a different type of capture molecule (in multiple copies), meaning that the capture molecule on the first layer is of a different type than the capture molecule on the second layer. For example, layer one may include an antibody that specifically binds a first target analyte and layer two may include a second antibody that specifically binds a second target analyte. Different may also mean that the capture molecules are different classes of molecules. For example, the first layer may include an antibody that binds a first target analyte and the second layer may include an aptamer that binds a second target analyte.

Metals can be conjugated to capture molecules (such as antibodies as shown in FIG. 19) in a variety of different ways. Up to 21 metals are known to have been conjugated (either covalently or non-covalently) to metals. This offers a large panel of antibody labels. An exemplary approach of coupling a metal to a capture molecule is described for example in Love et al. (Biochemistry. 1993 Oct. 19;32(41):10950-9), Meares et al. (International Journal of Radiation Applications and Instrumentation. Part B. Nuclear Medicine and Biology, Volume 13, Issue 4, 1986, Pages 311-318); Corneillie et al. (Journal of Inorganic Biochemistry, Volume 100, Issues 5-6, May 2006, Pages 882-890), and Torchilin et al. (Crit Rev Ther Drug Carrier Syst. 1991; 7(4):275-308), the content of each of which is incorporated by reference herein in its entirety.

Aspect of the methods herein leverage the different materials that make-up the reagents described herein. Bio-tags (such as gold, silver and latex particles) are used in association with bio-detection molecules (such as antibodies) to detect analytes because they have distinct physical properties. In an example, gold nanoparticles conjugated to antibodies are used because the gold nanoparticles can be visually detected (i.e., seen by the naked eye on a surface). That allows a user to identify where to direct the laser.

The reagent then further includes a capture molecule (e.g., antibody) complexed to a metal-bearing polymer. Capture molecules complexed to metal-bearing polymers can't be detected visually (e.g., by the naked eye) like gold or silver nanoparticles. These types of bio-tags require a different type of detection technique, such as described herein.

Metals complexed to antibodies offer a broad diversity of labels because each metal produces a very unique and narrow signal when analyzed with mass or atomic spectroscopy. Since mass spectroscopy is a very bulky mode of detection, the invention preferably uses atomic spectroscopy such as LIBS to detect metal-conjugated antibodies.

In certain embodiments, the metal particle conjugated to the capture molecule is a lanthanide. Exemplary metal particles may be composed of one or a combination of any of silicon, iron, zinc, silver, cadmium, indium, platinum, gold, lanthanum, praseodymium, neodymium, samarium, europium, gadolinium, terbium, dysprosium, holmium, erbium, thulium, ytterbium, and/or lutetium. Exemplary biomolecular labels, including metal particles, are shown in FIG. 25.

A wide range of samples (e.g., heterogeneous or homogeneous samples) can be analyzed, such as biological samples, environmental samples (including, e.g., industrial samples and agricultural samples), and food/beverage product samples, etc.

Exemplary biological samples include a human tissue or bodily fluid, which may be collected in any clinically acceptable manner. A tissue is a mass of connected cells and/or extracellular matrix material, e.g. skin tissue, hair, nails, nasal passage tissue, CNS tissue, neural tissue, eye tissue, liver tissue, kidney tissue, placental tissue, mammary gland tissue, placental tissue, mammary gland tissue, gastrointestinal tissue, musculoskeletal tissue, genitourinary tissue, bone marrow, and the like, derived from, for example, a human or other mammal and includes the connecting material and the liquid material in association with the cells and/or tissues. A body fluid is a liquid material derived from, for example, a human or other mammal. Such body fluids include, but are not limited to, mucous, blood, plasma, serum, serum derivatives, bile, blood, maternal blood, phlegm, saliva, sputum, sweat, amniotic fluid, menstrual fluid, mammary fluid, peritoneal fluid, urine, semen, and cerebrospinal fluid (CSF), such as lumbar or ventricular CSF. A sample may also be a fine needle aspirate or biopsied tissue. A sample also may be media containing cells or biological material. A sample may also be a blood clot, for example, a blood clot that has been obtained from whole blood after the serum has been removed.

In one embodiment, the biological sample can be a blood sample, from which plasma or serum can be extracted. The blood can be obtained by standard phlebotomy procedures and then separated. Typical separation methods for preparing a plasma sample include centrifugation of the blood sample. For example, immediately following blood draw, protease inhibitors and/or anticoagulants can be added to the blood sample. The tube is then cooled and centrifuged, and can subsequently be placed on ice. The resultant sample is separated into the following components: a clear solution of blood plasma in the upper phase; the buffy coat, which is a thin layer of leukocytes mixed with platelets; and erythrocytes (red blood cells). Typically, 8.5 mL of whole blood will yield about 2.5-3.0 mL of plasma.

Blood serum is prepared in a very similar fashion. Venous blood is collected, followed by mixing of protease inhibitors and coagulant with the blood by inversion. The blood is allowed to clot by standing tubes vertically at room temperature. The blood is then centrifuged, wherein the resultant supernatant is the designated serum. The serum sample should subsequently be placed on ice.

Prior to analyzing a sample, the sample may be purified, for example, using filtration or centrifugation. These techniques can be used, for example, to remove particulates and chemical interference. Various filtration media for removal of particles includes filer paper, such as cellulose and membrane filters, such as regenerated cellulose, cellulose acetate, nylon, PTFE, polypropylene, polyester, polyethersulfone, polycarbonate, and polyvinylpyrolidone. Various filtration media for removal of particulates and matrix interferences includes functionalized membranes, such as ion exchange membranes and affinity membranes; SPE cartridges such as silica- and polymer-based cartridges; and SPE (solid phase extraction) disks, such as PTFE- and fiberglass-based. Some of these filters can be provided in a disk format for loosely placing in filter holdings/housings, others are provided within a disposable tip that can be placed on, for example, standard blood collection tubes, and still others are provided in the form of an array with wells for receiving pipetted samples. Another type of filter includes spin filters. Spin filters consist of polypropylene centrifuge tubes with cellulose acetate filter membranes and are used in conjunction with centrifugation to remove particulates from samples, such as serum and plasma samples, typically diluted in aqueous buffers.

Filtration is affected in part, by porosity values, such that larger porosities filter out only the larger particulates and smaller porosities filtering out both smaller and larger porosities. Typical porosity values for sample filtration are the 0.20 and 0.45 μm porosities. Samples containing colloidal material or a large amount of fine particulates, considerable pressure may be required to force the liquid sample through the filter. Accordingly, for samples such as soil extracts or wastewater, a pre-filter or depth filter bed (e.g. “2-in-1” filter) can be used and which is placed on top of the membrane to prevent plugging with samples containing these types of particulates.

In some cases, centrifugation without filters can be used to remove particulates, as is often done with urine samples. For example, the samples are centrifuged. The resultant supernatant is then removed and frozen.

After a sample has been obtained and purified, the sample can be analyzed to determine the concentration of one or more target analytes, such as elements within a blood plasma sample. With respect to the analysis of a blood plasma sample, there are many elements present in the plasma, such as proteins (e.g., Albumin), nucleic acids, vitamins, hormones, and other elements (e.g., bilirubin and uric acid). Any of these elements may be detected using methods of the invention. More particularly, methods of the invention can be used to detect molecules in a biological sample that are indicative of a disease state. The target analyte(s) may then be quantified and correlated to a particular disease state, such as a cancer or other disorder.

A target analyte is the molecule in the sample to be captured, detected, and optionally quantified and correlated with an outcome or disease state. In certain embodiments, the sample in a biological sample. In such embodiments, the target analyte may be a target biological molecule in the sample (although the invention includes capturing non-biological molecules from a biological sample, such as a drug or a chemical substance). Examples of biological target analyte includes include proteins, nucleic acids (DNA and/or RNA), hormones, vitamins, bacteria, fungi, viruses, a cell (such as a cancer cell, a white blood cell a virally infected cell, or a fetal cell circulating in maternal circulation), and any biological molecules known in the art and typically found in a biological sample.

A capture molecule refers to a molecule that specifically binds a target analyte from the sample. The capture molecule chosen will depend on the target analyte to be captured and one of skill in the art will readily be able to select the capture molecule to use based on the desired target analyte to be captured and analyzed. Exemplary capture molecules include antibodies, nucleic acids (DNA or RNA), peptides, proteins, aptamers, receptors, ligands, etc.

In particular embodiments, the capture molecule is an antibody. The term antibody includes complete antibodies and any functional fragment of an antibody that can specifically bind a target analyte. General methodologies for antibody production, including criteria to be considered when choosing an animal for the production of antisera, are described in Harlow et al. (Antibodies, Cold Spring Harbor Laboratory, pp. 93-117, 1988). For example, an animal of suitable size such as goats, dogs, sheep, mice, or camels are immunized by administration of an amount of immunogen, such the target bacteria, effective to produce an immune response. An exemplary protocol is as follows. The animal is subcutaneously injected in the back with 100 micrograms to 100 milligrams of antigen, dependent on the size of the animal, followed three weeks later with an intraperitoneal injection of 100 micrograms to 100 milligrams of immunogen with adjuvant dependent on the size of the animal, for example Freund's complete adjuvant. Additional intraperitoneal injections every two weeks with adjuvant, for example Freund's incomplete adjuvant, are administered until a suitable titer of antibody in the animal's blood is achieved. Exemplary titers include a titer of at least about 1:5000 or a titer of 1:100,000 or more, i.e., the dilution having a detectable activity. The antibodies are purified, for example, by affinity purification on columns containing hepatic cells.

The technique of in vitro immunization of human lymphocytes is used to generate monoclonal antibodies. Techniques for in vitro immunization of human lymphocytes are well known to those skilled in the art. See, e.g., Inai, et al., Histochemistry, 99(5):335 362, May 1993; Mulder, et al., Hum. Immunol., 36(3):186 192, 1993; Harada, et al., J. Oral Pathol. Med., 22(4):145 152, 1993; Stauber, et al., J. Immunol. Methods, 161(2):157 168, 1993; and Venkateswaran, et al., Hybridoma, 11(6) 729 739, 1992. These techniques can be used to produce antigen-reactive monoclonal antibodies, including antigen-specific IgG, and IgM monoclonal antibodies.

Methods for attaching the capture molecule, such as an antibody, to a particle core are known in the art. Coating magnetic particles with antibodies is well known in the art, see for example Harlow et al. (Antibodies, Cold Spring Harbor Laboratory, 1988), Hunter et al. (Immunoassays for Clinical Chemistry, pp. 147-162, eds., Churchill Livingston, Edinborough, 1983), and Stanley (Essentials in Immunology and serology, Delmar, pp. 152-153, 2002). Such methodology can easily be modified by one of skill in the art to bind other types of capture moieties to particles. Certain types of particles coated with a capture molecule are commercially available from Sigma-Aldrich (St. Louis, Mo.).

Reference to binding of a target analyte to a capture molecule refers to members of a specific binding pair (or binding partners), which are moieties that specifically recognize and bind each other. Specific binding pairs are exemplified by a receptor and its ligand, enzyme and its substrate, cofactor or coenzyme, an antibody or Fab fragment and its antigen or ligand, a sugar and lectin, biotin and streptavidin or avidin, a ligand and chelating agent, a protein or amino acid and its specific binding metal such as histidine and nickel, substantially complementary polynucleotide sequences, which include completely or partially complementary sequences, and complementary homopolymeric sequences. Specific binding pairs may be naturally occurring (e.g., enzyme and substrate), synthetic (e.g., synthetic receptor and synthetic ligand), or a combination of a naturally occurring BPM and a synthetic BPM.

Target capture refers to selectively separating a target analyte from other components of a sample mixture, such as cellular fragments, organelles, proteins, lipids, carbohydrates, or other nucleic acids. Target capture as described herein means to specifically and selectively separate a predetermined target analyte from other sample components, e.g., by using a target specific molecule.

In certain embodiments, the directing and detecting steps of the methods of the invention described herein are accomplished one or more laser based systems, such as using Laser-Induced Breakdown Spectroscopy (LIBS). In certain embodiments, a single laser based system is employed. In certain embodiments, combinations of different laser based systems are contemplated. Laser-induced breakdown spectroscopy (LIBS) is a type of atomic emission spectroscopy which uses a highly energetic laser pulse as the excitation source. The laser is focused to form a plasma, which atomizes and excites samples. Spark-induced breakdown spectroscopy (SIBS) is a plasma-based atomic emission analytical technique that draws from both traditional spark spectroscopy and laser-induced breakdown spectroscopy (LIBS).

Exemplary LIBS systems are shown in FIGS. 1, 2, and 23. Typically, LIBS systems are composed of 3 base components: a pulsed laser (such as a 100uJ cobalt laser), laser focusing optics, point-source collection optics, and a spectrometer (with a CCD or ICCD detector). In certain embodiments, LIBS is performed in air, argon and helium environments to optimize plasma production.

Laser-induced breakdown spectroscopy (LIBS) is a sample characterization technique based on the production and analysis of the fourth state of matter-ionic plasma. Plasmas produced during LIBS emit complex optical emissions consisting of a continuous background spectrum and discrete line emissions representative of the elemental components of the sample. When an energy pulse is applied to a solid substrate, the atoms in or near the path of the energy pulse are heated. If the heating is sufficient, the energy pulse is followed by a visible flash and popping sound generated by the rapid expansion of hot material and air. The expanding ionized gas is plasma, the fourth state of matter. The fraction of material that reaches the plasma-electron temperature threshold (˜10 eV) forms a plume along the energy pulse path. Based on the spectral emission properties of the plume, one can characterize the composition of the source material. The nature of plasma formation and emission detection is highly dependent on certain parameters: (i) mode of induction, (ii) pulse duration, (iii) repetition rate, (iv) laser wavelength (if a laser is used), (v) time of analysis, (vi) environmental temperature, pressure, and atomic composition, (vii) physical properties of the substrate, and (viii) spatial distribution of the plasma. The effects of these parameters on plasmas can be explained by the physical principles of thermal and non-thermal energy absorption and dissipation over time.

LIBS is further described for example in Anabitarte et al. (ISRN Spectroscopy 2012:12, 2012); and Aragon et al. (Applied Spectroscopy 51(11):1632-1638, 1997), the content of each of which is incorporated by reference herein in its entirety.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes. The content of WO 2020/056257 is also expressly incorporated by reference herein in its entirety.

### EQUIVALENTS

The invention may be embodied in other specific forms without departing from the spirit or essential characteristics thereof. The foregoing embodiments are therefore to be considered in all respects illustrative rather than limiting on the invention described herein.

### EXAMPLES

### Example 1: Food Authentication and Fingerprinting Method for Food Fraud Prevention and Tracing

LIBS is based on atomic emission spectroscopy, using a laser that ablates a tiny amount of the analyte to produce a plasma plume. Upon cooling of plasma, energy is emitted, and the acquired spectra describe the sample composition. Competing methods, such as X-ray fluorescence spectrometry (XRF) or ICP MS, are costly, produce large amounts of toxic waste, require expensive reagents and fume hoods. The disclosed method utilizing LIBS paired with machine learning demonstrates the use of fingerprinting for classification and authentication of a closely related and similar product belonging to three types of food categories (coffee beans, balsamic vinegar, and hard cheeses). These disclosed methods may be further validated for other foods such as meat, fish, and fresh vegetables.

Although a number of researchers published proof-of-concept manuscripts arguing for the use of LIBS in the ag industry, fingerprinting applications have not been disclosed or pursued. There are multiple reasons: (1) lack of food-optimized instrumentation, especially portable LIBS systems (as all the commercially available handheld LIBS systems were developed for material science, in particular, metal alloy analysis or soil research) limited the ability to create a functional embodiment, and (2) inadequate data processing tools, typically relying on standard spectroscopic approaches focused on elemental analysis rather than fingerprinting which employs large, diverse open-ended libraries. This disclosure demonstrates the method that addresses these two significant shortcomings.

### LIBS Instrumentation

The ranges of optimal LIBS detection vary depending on the examined material. For instance, a problem of cadmium contamination in vegetables requires only a narrow spectral range of 200-250 nm, whereas an assessment of chemical elements in chicken meat and beef needs two spectrometers covering a range from 250 to 1100 nm. Our research demonstrated that commercial alloy testing devices used in material science offer sufficient laser power for food fingerprinting; however, the detection subsystem has often been lacking sensitivity or resolution in the spectral regions important for food analysis. In our prototype, we performed food product fingerprinting using a 360-650 nm range, achieving overall excellent practical accuracy (measured as AUC>0.9).

This performance is achieved with spectra showing well-resolved features, given the fact that the food fingerprinting measures may be executed in an environment outside of the established laboratories. With field-deployable instruments, it is important that the data is statistically evaluated before future processing to ensure that the downstream data processing uses high-quality spectra amenable to automated processing and machine learning application. One possible approach is defining a spectral input “figure-of-merit,” for instance, as a ratio between the variance and the maximum intensity of the observed elemental peak. If the observed ratio drops below a particular value, the observation may be rejected as inconsistently informative.

The instrument optimization also includes setting optimal laser spot size (ranging from 20 to 500 μm), optimal laser energy per pulse, as well as measurement delay time. These values were established by performing a grid search in the space of all these configuration parameters, in which the ROC of the downstream classifier is considered to be the guide. The set of optimization searches must be executed for all the fingerprinted food groups.

### Sample Measurement

The sample handling and measurement are dictated by the examined material. We have demonstrated that the cheese authentication can be performed directly with the food samples. The liquid products such as vanilla extracts or balsamic vinegar were deposited on cellulose strips. The spices and other powdered substances can be examined utilizing pre-formed pellets following the existing protocols for soil analysis.

### Data Processing and Spectral Analysis

The established LIBS data analysis uses traditional chemometrics employing spectral normalization and denoising followed by matrix algebra tools and peak identification. Our method recognized the fact that the differences in individual peaks and the interpretability of these differences in the context of complex food matrices may be difficult and may not lead to satisfactory accuracy of classification. Therefore we use a non-targeted detection approach, in which extensive use of automated feature selection and classification tools takes under consideration the entire spectral fingerprint.

Our method incorporates the following steps:
Spectral baseline adjustment and correction followed by filtering/denoising to improve the signal-to-noise ratio of the collected spectra, and normalization
Univariate feature filtering employing generalized linear models
Multivariate feature selection and classification using regularized regression
Optional additional classification step using machine learning methodologies such as support vector machine or neural network.

In the preferred embodiment, we use an elastic-net feature selection model employing combined LASSO and ridge penalties. Since the number of possible spectral fingerprint features in LIBS signal is expected to be relatively high, and many of them may convey potentially valuable information regarding sample characteristics, we prefer to use ante-hoc explainable models rather than black-box approaches (such as deep learning) that require complex post-processing procedures to establish explainability. Therefore, in one embodiment, we implemented a regularized multinomial regression model regularized via elastic net penalty, trained with a wide selection of agricultural products. The model is represented as:

$$log \frac{Pr ( {y}_{i} = k \vert x )}{Pr ( {y}_{i} = K \vert x )} = {b}_{k} + {w}_{k}^{T} x , ( k = 1 , 2 , … , K - 1 ) ,$$

where wₖ is a k^{th}-row vector in the parameter matrix, and b=(b₁, . . . , bₖ)^{T} is the bias. Accordingly, (bₖ, wₖ) is a pair of parameters that corresponds to sample Y=k|x (instance × of a sample belonging to class k), and bₖ∈ [symbol] , wₖ∈ [symbol] ^{p} (p is the number of network descriptors). This formulation leads to an optimization problem:

$$arg {min}_{( b , w )} \{ - \frac{1}{n} {\sum }_{i = 1}^{n} [ {\sum }_{k = 1}^{K} {y}_{ik} ( {b}_{k} + {w}_{k}^{T} {x}_{i} ) - log {\sum }_{k = 1}^{K} {e}^{( {b}_{k} + {w}_{k}^{T} {x}_{i} )} ] + λ [ α {\sum }_{k = 1}^{K} {\sum }_{j = 1}^{p} {w}_{kj}^{2} + ( 1 - α ) {\sum }_{k = 1}^{K} {\sum }_{j = 1}^{p} \vert {w}_{kj} \vert ] \}$$

where α, λ≥0 are tuning parameters for the penalty term found via grid-search and cross-validation. As the result of the above process, the system provides a simple predictive classifier, as well as selections of spectral features, which determine the classifier's decision. Optionally, the top features may be further used in another classifier of choice, such as SVM.
The classification process may provide standalone providing the final classification result or may be incorporated into an expanded classification pipeline employing multi-view learning paradigms, where other set of features can be collected from other spectroscopic (e.g., Raman spectroscopy, FTIR spectroscopy) or non-spectroscopic evaluation of the food samples using complementary biophysical testing methods.

### Tested Food Groups

LIBS has been shown to perform measurements on variety of agricultural commodities including tea, coffee, honey, butter, milk, cereal, and olive oils. In our work, we utilize LIBS as the source of data for authentication of cheese, coffee, olive oil, vanilla extract, and spices. We utilize full spectral fingerprints rather than elemental analysis, as it is conventionally done with LIBS.

### Example 2: Rapid 15-Minute Libs-Based Assay for Monitoring Onset of Cytokine Storm in Covid-19 Infection

One of the goals of this study was to determine if some molecules could be measured in a time frame of less than 30 minutes. Previously, we have developed an assay technology that uses laser induced breakdown spectroscopy (LIBS) to measure antibodies conjugated to lanthanides (Carmen Gondhalekar et al., 2020). Other biologically related studies using LIBS have demonstrated detection of cancer markers based on detection of an antibody-bead assay (Markushin et al., 2009), detection of pathogens in food (Barnett et al., 2011) aas well as evaluation of molecular composition of infant formulas (Abdel-Salam, Al Sharnoubi, & Harith, 2013). Some of the primary advantages of LIBS include its speed of operation, its ability to analyze very small volumes of sample and the ability to work with both solid and liquid samples.

### Materials and Reagents

Capture antibody used in the LFIAs (Ab1, 2 mgmL⁻¹, cataloge #: 93896) was purchased from BioLegend (San Diego, Calif., USA). Antibody for detection of IL-6 (Ab2, 2.0 mg mL-1, cataloge #: 1-150) was from Leinco (St. Louis, Mo., USA). Goat anti-rabbit IgG and rabbit anti-goat IgG were from Invitrogen (Waltham, Mass.). The IL-6 was obtained from Leinco (St. Louis, Mo., USA). Chemicals used to prepare 0.01M phosphate-buffered saline (PBS, pH 7.4) and Tween-20 were from Sigma-Aldrich (St. Louis, Mo.). Albumin Bovine Serum (BSA) were purchased from GoldBio (St Louis Mo.). The Vivid120 nitrocellulose (NC) membrane was from Pall Corporation (New York, N.Y., USA). The FF170HP Plus and the absorbent pad CF6 were from GE Healthcare (Chicago, Ill., USA). The standard gold nanoparticles of different size (GNPs, OD=1, 40 nm; OD=1, 20 nm) were from Cytodiagnostics (Burlington, ON, Canada). The water was deionized and ultrafiltered using a Milli-Q (what model?) apparatus.

### Experimental Instruments

The optimization of test and experimental line dispensing parameters was performed to achieve optimal amount of capture antibodies, including the syringe pump rate, dispensing rate, dispensing length and air pressure using a BioJet Quant ZX1000 dispenser (Biodot Ltd. (Irvine, Calif., USA).

The benchtop LIBS instrument is described in detail in Gondhalekar et al. 2020 (C. Gondhalekar et al., 2020), consisted of a 1064-nm 4-ns pulsed laser (Nano SG 150-10, Litron Lasers, Bozeman, Mont., USA) with a 150-mJ maximum laser pulse and 10-Hz maximum repetition rate. For experimentation, 35 mJ of pulse energy and a spot size of ˜700 μm were used. A spectrometer and ICCD from Andor Technologies (SR-5001-B1 and DH320T-18F-E3) were used to measure spectra and control integration time, which was maintained at 500 ns throughout the study. Ablations took place in a custom designed chamber fitted with a vacuum pump and air filter to remove hazardous aerosols. Pressure inside the chamber was maintained at 1 atm. The chamber was supported by a XYZ stage (TPA0348B-00, The Precision Alliance, Fort Mill, S.C., USA) that permitted pre-programmed and automated movement of samples. Coupling the timing between laser pulses (10 Hz) and automated stage movement allowed for rapid sampling.

Conjugation and Characterization of Gold Nanoparticles (GNPs) with Antibody

Antibodies for detection of IL-6 (Ab2) were passively absorbed onto the surface of gold nanoparticles and applied to the test strips. Absorption reactions were performed at different pH and antibody concentrations. The optimal treatment was applied to test strips.

W e employed ultraviolet-visible spectrometer (UV-vis, Synergy H1 multi-mode reader, BioTek Instruments, Winooski, Vt.) and Nanosight dynamic light scattering analyzer (LM10, Malvern Panalytical Ltd, Malvern, United Kingdom) for the characterization of GNPs conjugated with antibody. Absorbance and size of unconjugated nanoparticles and storage buffer were also measured as controls.
Preparation of Europium-Complexed Polymer and Conjugates with Antibody

20 μL of 0.5 mg/mL ab2 were conjugated to 151Eu by linking of Eu-complexed polymers using Fluidigm's MaxPar ×8 kits (Fluidigm, 201151A, San Francisco, Calif.). The protocol recommended by Fluidigm (Fluidigm) was employed with modifications. In brief, 95 μl of proprietary L buffer from the conjugation kit was added to one polymer tube, then transferred to another polymer tube. 10 μl of metal supplied by the kit's metal stock solution was then added to the polymer mixture. Following these initial steps, standard Fluidigm procedure was followed until step 32 (Fluidigm), where the antibody was suspended for recovery. In the process of washing the metal-polymer solution using centrifugal filtration, the reaction solution containing the metal went through six wash steps. After the sixth wash (step 31) (Fluidigm), 100 μl buffer was used to wash the walls of the centrifugal filter unit. Each filter wall was washed 10 times without touching the filter membrane with the pipette tip. The unit was inverted into a microcentrifuge tube and spun at 1000×g (for how long). The wash, inversion, and centrifugation steps were repeated using an additional 100 or 200 μl buffer. The final volume of antibody suspension was 200-320 μl. After conjugation was complete, antibody concentration was measured using a NanoDrop One (Thermo Fisher Scientific, Waltham, Mass., USA). The final product was diluted with antibody stabilizer (Candor Bioscience, Wangen, Germany) and 0.2% sodium azide.

### Design and Fabrication of LFIAs

Since our bioassay chemistry and bio-labels have been well characterized, the only remaining variable to control for device performance and flow dynamic was the porosity and geometry of NC membrane, which is at the core of the LFIA devices. Various types of NC membranes (HF120, HF170, CN 95, CN 140, CN150) were tested with GNPs conjugates and Eu conjugates. HF120 and HF170 (also referring as NC120 and NC170) were selected for the following experiments, which exhibit the best fit for the design requirement of rapidity and sensitivity. As follows, we investigated and characterized influence of the width (w) and length (1) of on the flow regime and analytical performance.

### Preparation of IL-6 Standards and Controls

A series of reference standards were set at 0, 0.5, 1, 2, 10, 20, and 40 ng/mL by diluting the IL-6 (0.1 mg/mL) with the dilution buffer.

### Preparation of Serum Samples

Serum samples were collected from healthy adults free of COVID-19. Different levels of IL-6 were spiked into the samples and were stored at −20° C. until use. The study was reviewed and approved by the clinical research ethics committee of Purdue University.

### Sample Detection and Analysis by LFIA-LIBS Biosensor

Initially, 20 μL of a sample (standard or serum) and 20 μL of sample dilution buffer were mixed thoroughly. A total of 90 μL of Eu-conjugated antibody was mixed with a sample. Ten minutes later, the mixture was introduced to the LFIA test strip for 5 min. The nitrocellulose portion of the test strip was separated from the waste pad and air-dried for 2 h. Two types of negative controls were used: the first underwent the same treatment as the experimental group, but PBS was used instead of IL-6; the second type of negative control was treated similarly to the experimental group, except that 90 μl PBS was used instead of 90 μL antibody conjugated to Eu.

For LIBS detection of the Eu-labeled IL-6 on the strip, the parameters determined to be optimal for Eu emission detection as previously published (C. Gondhalekar et al., 2020) were applied. The test line and control line were each shot 8 times in 8 locations per strip. The series of reference standards (0, 0.5, 1, 2, 10, 20, and 40 ng/mL) were set for standard curve making and signal-to-noise ratio (SNR) measuring.

### Data Analysis

LIBS spectra were analyzed using a custom-developed procedure written in R language for statistical computing (R), described in detail in Gondhalekar et al. 2020 (C. Gondhalekar et al., 2020). In brief, a sliding median filter estimated the background across the wavelength range and was subtracted from the raw data. To determine signal-to-noise ratio (SNR), the data were then standardized by dividing by the standard deviation of the noise, estimated using a second median filter. This process was repeated for every spectrum acquired with LIBS.

Limit of detection was determined by applying the following formula to a dilution series of the metal standard:

LOD=((3.3*SD₀+μ₀)−b)/m

Where SD₀ is the standard deviation of the SNR in the area adjacent to the test line, to is the mean SNR of the emission line in the negative control, b is the y-intercept of the regression line, and m is the slope of the regression line. The regression line equation was derived from a linear fit of the SNR vs. concentration data for each analyte. To obtain a linear fit for the lanthanide dilution series, both axes were log-transformed.

Results

### Establishment of New Detection System and Data Evaluation

Antibody-Tagging with GNPs for Direct Visualization

We labeled anti-human IL-6 pAbs with 20 nm and 40 nm GNPs. The immunoreaction between GNPs-Ab2 (label antibody, anti-human IL-6 pAbs), IL-6 and mAbs (capture antibody, anti-human IL-6 mAbs) resulted in the accumulation of GNPs on the test lines of the LFIA. The combination of excess GNPs-pAbs and Rabbit anti-goat IgG on the control (C) line ensured the validity of the LFIA detection. Pink positive test lines and positive control lines were visualized on test strips when samples containing IL-6 were introduced into the LFIA devices. The intensity of control and test lines was higher for antibodies conjugated to 40 nm GNPs, likely because they have larger surface area for conjugation and less interruption from background.

Antibody-tagging with Lanthanides

Eu was covalently conjugated to anti-human IL-6 pAbs and Yb was covalently conjugated to anti-human IP-10 pAbs using Fluidigm's metal conjugation kit (Fluidigm, 201151A, San Francisco, Calif.). The initial input of 100 μg antibody to the reaction results in a higher recovery rate of antibody characterized by NanoDrop compared to 50 μg of initial input of antibody. There was 13.20 μg Eu conjugated on 100 μg of anti-human IL-6 pAbs and 28.93 Yb conjugated on 100 μg of anti-human IL-6 pAbs. The successful conjugations indicate that Eu-pAbs and Yb-pAbs were successfully prepared and could be effectively employed for LFIA-LIBS detection of cytokines. After conjugation, the mixture was prepared and introduced to the LFIA test strips. Afterwards, LFIA test strips can be directly subjected to LIBS analysis without any pretreatment, in which the Eu and Yb elements are ionized and the signal intensity of Eu (II) (the peak at 420.504 nm) and Yb (II) at 369.419 nm are chosen for quantitative analysis due to its higher intensity compared to its other characteristic wavelengths.

### Construction of the LFIA Devices for Cytokines Detection

Since our bioassay chemistry and bio-labels have been well characterized, the only remaining variable to control for device performance and flow dynamic was the porosity and geometry of NC membrane, which is at the core of the LFIA devices. Various types of NC membranes (HF120, HF170, CN 95, CN 140, CN150) were tested with GNPs conjugates and Eu conjugates. HF120 and HF170 (also referring as NC120 and NC170) were selected for the following experiments, which exhibit the best fit for the design requirement of rapidity and sensitivity. As follows, we investigated and characterized influence of the width (w) and length (1) of on the flow regime and analytical performance.

### LIBS Dose-response of Lanthanides-labeled Cytokine Standards and Determination of LODs

We investigated the sensitivity of the LIBS-LFIA sensor for detection of IL-6. The Eu intensity of T lines on the strips progressively strengthened with the increasing concentration of IL-6 standards from 0 to 1.2 μg/mL, giving a linear correlation (Y=2.6299X+2.9607, R²=0.98) in the range of 0.01 to 1.2 μg/mL. The limit of detection (LOD) of the LFIA-LIBS sensor was estimated to be 0.2298 μg/mL, which was defined as 35/M (S=0.2014, M=2.6299, where S was the value of the standard deviation of the blank samples and M was the slope of the standard curve within the linear range of the low concentrations). To ensure the signal reproducibility, eight different laser spots on the T line were chosen to yield an average LIBS signal for one single test strip.

### Optimization of Reaction Parameters

### The Optimum Amount of Capture Antibody

The captured antibody was diluted to 2.0 mg/mL with coating buffer. Two different sprayed speeds were set to optimize the better quantity of capture antibody. In plan A, anti-Goat IgG (1 mg/mL) was sprayed onto the control line (C) at a speed of 1 μL/mm, while the capture antibody was sprayed onto the test line (T) at a speed of 0.5 μL/mm. In plan B, anti-Goat IgG was handled the same as in plan A, but capture antibody was sprayed onto T line at a speed of 1 μL/mm. Plan A was chosen based on better linearity and continuity.

### The Optimum Concentration of Conjugates

When the optimized sprayed speed was fixed at 1 μL/mm for both capture antibodies on the T and C line, different concentrations (0.01 mg/ml, 0.1 mg/ml, and 1 mg/ml) were set as the optimum choice of conjugates at the speed of 1 μL/mm. The series of reference standards (0, 0.5, 2, 10, 20, and 40 ng/mL) were used here for measuring the signal-to-noise ratio (SNR).

### DISCUSSION

We developed a new sensing format of applying lateral flow immunoassays (LFIAs) and an effective combination of LFIA and laser induced breakdown spectroscopy (LIBS) to rapidly detect cytokines. Elevated serum Interleukin 6 (IL-6) and Interferon gamma-induced protein 10 (IP-10) correlate with respiratory failure, acute respiratory distress syndrome (ARDS), and adverse clinical outcomes in COVID-19 patients, which are biomarkers of severe beta-coronavirus infection. Using Protect Purdue funding, we developed approaches to evaluate the quantitative performances of this rapid cytokine assay; we performed the detection of IL-6 and IP-10 as model applications using this assay technique. We tagged anti-human IL-6 antibodies and anti-human IP-10 antibodies with gold nanoparticles (GNPs) and lanthanides respectively. Direct visualization of using antibody conjugated AuNPs as the label confirmed the design and feasibility of detecting cytokines in LFIA device. In our previous study we found that europium (Eu) and ytterbium (Yb) may be more favorable biomolecular labels than Au for spectroscopic analysis using LIBS. Thus, Eu was conjugated to anti-human IL-6 polyclonal antibodies (pAbs) and Yb was conjugated to anti-human IP-10 polyclonal antibodies. They were selected as the signal generators for LIBS detection.

Here we introduce a new dimension for LFIA design and optimization based on geometric flow control (GFC) of nitrocellulose (NC) membranes, leading to highly sensitive LFIA. This novel approach enables comprehensive flow control via different membrane geometric features such as the width (w) and the length (1). Our new development on GFC-LFIA devices, tailored flow control and improved analytical performance as well as reduced antibody consumption. Moreover, selection of specific NC membranes in LFIA devices was also identified as a critical component. NC170 and NC120 membranes were selected and optimized to be suitable for our LFIA-LIBS detection of cytokines. Subsequently, we investigated the sensitivity of the LFIA-LIBS sensor for cytokine detection and analysis. The bench-based LIES system was optimized for the detection of lanthanides including Eu and Yb. We studied the LIBS dose-response of IL-6 standards and estimated the limit of detection (LOD) of our LFIA-LIBS sensor. In conclusion, our method can be finished within 15-minute and reach a detection limit of 0.2298 μg/mL, showing an effective collaboration of LIBS and LFIA that is promising for rapid and accurate detection of cytokines in clinical diagnosis of COVID-19 and any patient in immune distress.

Reproducibility, specificity and stability of the LFIA-LIBS sensor are key parameters for successful rapid cytokine assay. To study the batch-to-batch variation, we applied the LIBS-LFS sensor for IL-6 detection using three test strips in parallel. However, five replicates of test trips could reduce the relative standard deviation (RSD) to be as low as possible, leading to a more reproducibility for cytokine detection. Specificity of our method for the detection of cytokine should be tested to prove high specificity of our LFIA-LIBS sensor for rapid cytokine detection. Besides the reproducibility and specificity, the potential of the LFIA-LIBS sensor for long-term data preservation is very promising. We expect to see there is no obvious decay observed for the LIES intensity during preservation and showing an acceptable RSD. The signal stability of our sensor could be advantageous for reliable tracking and comparison of the detection results throughout desired time points of diagnosis.

### CONCLUSIONS

A rapid, sensitive, quantitative LFIA-LIBS biosensor for detection of cytokines in urgent clinical environment has been developed and optimized to the desired results. Compared to the existing detection technologies, our research work employs lanthanides chelated polymers to link lanthanides to antibodies into the method that LFIA use gold nanoparticles as a visualization label for detection of analytes, which produces distinctive analytical performance to guarantee high sensitivity and accuracy. The combination of LFIA and LIBS provides rapid and improved detection performance to quantify cytokine levels, as well as being cost-effective. To offset the interference and non-uniformity caused by the sample matrix and test strips, the SNR was calculated, and LFIA-LIBS biosensor was optimized for effectiveness evaluation. New LFIA design based on geometric flow control (GFC) lead to increased sensitivity of paper-based assays.

Characterization and optimization of antibody conjugation to gold nanoparticles and lanthanide-bearing polymers as well as reaction parameters has been achieved. Summarily, we successfully labeled cytokines and applied LIBS for rapid detection of cytokine on a lateral flow assay. Our research provides evidence that rapid and accurate detection of cytokines for clinical diagnosis and prognosis of COVID as well as using LIBS is highly feasible and compatible with the POC format.

Measurement of molecules has been achieved using many different approaches. For example, ELISA, flow cytometry, chemiluminescence have all been used successfully. However one of the key aspects of molecule analysis is time to achieve result. For ELISA a typical assay requires several hours for completion, even when the fastest techniques are used. In our experience using bead-based flow cytometry assays, time to result can exceed 12 hours because of the number of washes and sample additions required.

When evaluating cytokines in particular, it is now well established that a cytokine storm has been identified as a complicating factor in Sars-CoV-2 disease (Song, Li, Xie, Hou, & You, 2020). Multiple cytokines have been identified as being associated with COVID disease including IL-1B, IL-6, IL1-12, TNF and IFN-g, IP-10, (Ruan, Yang, Wang, Jiang, & Song, 2020), (Song et al., 2020). While cytokines are normally induced in most inflammatory situations, excessive or continuous cytokine production can cause tremendous tissue damage (Liu et al., 2020).

Over the past several years, there has been an increase in the availability and utility of handheld instruments including photoacoustic imagers (Liu S Fau-Feng et al., 2019), XRF (Simsek Franci, 2020), X-ray diffraction (Hansford, 2018), Spectroscopy (Crocombe, 2018), Raman Spectrometer (Owens et al., 2018), handheld optical coherence tomography (Jung et al., 2011), fluorescence (Ranieri et al., 2014), and LIBS (Connors, Somers, & Day, 2016; Cremers et al., 2012; Crocombe, 2018; Erler, Riebe, Beitz, Löhmannsroben, & Gebbers, 2020; Kim et al., 2019; Manard, Wylie, & Willson, 2018).

The advantages of handheld devices are significant since they can be taken on site, used in locations where large equipment cannot be located, they can provide results faster if samples do not have to be transported.

### Example 3: Multivariable Classification

Sample Preparation

6 different types of Vanilla (Table 1)
Measured date: 01/15/21 & 02/15/21//05/26/2021
Total number of spectrum: n=883//n=798

TABLE 1

| | |
|---|---|
| V1 | Pure Vanilla extract, Kroger |
| V2 | Imitation Vanilla flavor, Kroger |
| V3 | Pure Vanilla extract, McCormick |
| V4 | Pure Vanilla from Mexico, San Luis Rey |
| V5 | Vanilla syrup, 1:2 dilution, Barman |
| V6 | Vanilla from Madagascar, Simple truth |

6 different types of Vinegar (Table 2)
Measured date: 01/12/21 & 02/15/21//05/27/2021
Total number of spectrum: n=768//n=647
1:1 dilution

TABLE 2

| | |
|---|---|
| V1 | Balsamic vinegar of modena, Italy |
| V2 | COIAVITA Balsamic vinegar of modena, Modena |
| V3 | Barrel aged balsamic vinegar, Napa Valley Harvest |
| V4 | Gran Deposito ACETO balsamico DI modena, GIUSEPPE GIUSTI, |
| | Modena |
| V5 | Trader Joe's Gold quality Balsamic Vinegar of Modena, Italy |
| V6 | From Andrea Cossanga own balsamic barrel |

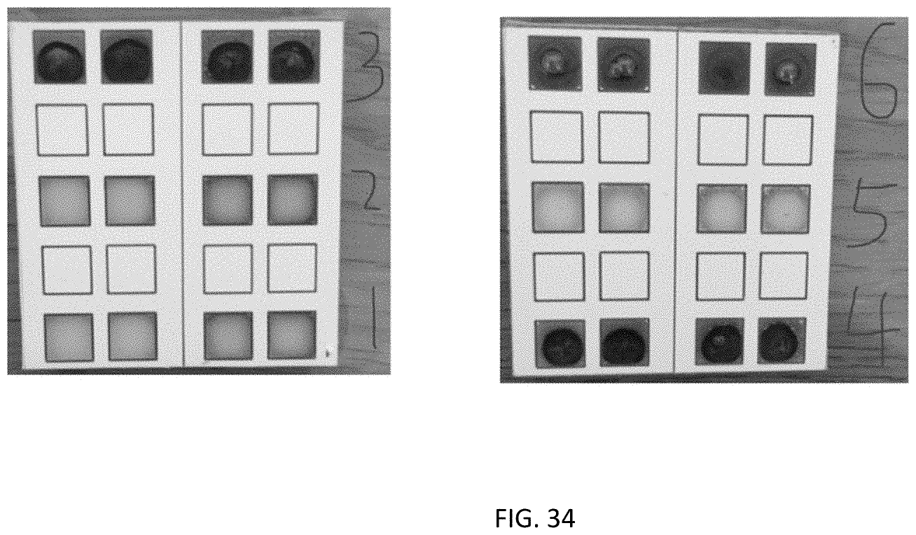

Liquid sample preparation (See FIG. 34)
Doping on Nitrocellulose paper (square dimension: 6*6 mm²)
Elements dissolved in nitric acid and dried onto paper
   7 different types of Coffee (Table 3)
Measured date: 02/23/21 & 03/10/21//05/19/21 (SciAps)
Total number of spectrum: n=482//n=1333
Laser was directly irradiated on the back surface of coffee bean

TABLE 3

| | |
|---|---|
| C1 | Italian roast Expresso |
| C2 | Copper Moon Light Roast Blend Guatemaria |
| C3 | Lavasa Super Geace |
| C4 | Despierta tus Sentidos |
| C5 | Mayorga Organics Café Cubano Roast |
| C6 | Koffee Kult dark roast |
| C7 | Verena Street |

16 different types of Cheese (Table 4)
4 sections (10*10 mm², 2 mm thick) per each cheese types

TABLE 4

| Code | Number | Item | Country | Na content |
|---|---|---|---|---|
| 108S | 1 | Frantal Emmental | France | 60 mg |
| 114S | 2 | Comte AOP 12 Months by | France | 0 mg |
| | | Charles Amaud | | |
| 120S | 2 | Appenzeller | Switzerland | 170 mg |
| 178S | 4 | Gruyere AOP | Switzerland | 160 mg |
| 268S | 5 | Abondance AOP | France | 144 mg |
| 815S | 6 | Kaltbach Cave Aged Swiss | Switzerland | 160 mg |
| | | Gruyere AOP | | |
| 858S | 7 | Kaltbach Cave Aged | Switzerland | 60 mg |
| | | Emmental AOP by Emmi | | |
| 1064 | 8 | Comte AOP 24 Months | France | 0 mg |
| | | aged by Charles Arnaud | | |
| 1598S | 9 | Austrian Alps Gruyere | Austria | 200 mg |
| 1852 | 10 | Bergzenusss | Switzerland | 157 mg |
| 2038 | 11 | Hoch Ybrig | Switzerland | 157 mg |
| 3953 | 12 | Comte AOP 10 Months | France | 90 mg |
| | | aged | | |
| 8742 | 13 | Brenta | Italy | 180 mg |
| 8907 | 14 | Comte AOP 6 Months aged | France | 105 mg |
| | | by Charles Amand | | |
| 1181S | 15 | Parpan Alpkaese | Switzerland | 110 mg |
| | 16 | Wisconsin | US | |

### Data set of Cheese

Bench-top instrument
03/30/21: n=973, 15 classes
04/13/21: n=1091, 16 classes
04/27/21: n=996, 16 classes
05/11/21: n=, 16 classes
05/25/21: n=, 16 classes
Hand-held instrument
04/13/21: n=1507, 16 classes
04/29/21: n=1566, 16 classes
05/11/21: n=1341, 16 classes
05/26/21: n=, 16 classes
italicized means filtered data
From filtering, —10% of data were removed
   8 different types of Spices (Table 5)
Measured date: 05/26/2021//06/01/2021
Total number of spectrum: n=556//n=769 (SciAps)

TABLE 5

| | |
|---|---|
| S1 | Indian nutmeg |
| S2 | Ground nutmeg |
| S3 | Mustard ground |
| S4 | Crushed red pepper |
| S5 | Cayenne pepper |
| S6 | McCormick ground cumin |
| S7 | Cumin ground |
| S8 | Turmeric ground |

3 different types of Olive oils (Table 6)
Measured date: 11/13/2020 & 01/06/2021
Total number of spectrum: n=101

TABLE 6

| | |
|---|---|
| S1 | Olive oil |
| S2 | Vegetable oil |
| S3 | BV |

Analytical Methods

Condition of 1st bench-top instrument (Table 7)

TABLE 7

| Laser | Nano SG 150-10 Nd: YAG 1064 nm laser (Litron Lasers, | |
|---|---|---|
| | USA) | |
| Pulse width | 4 ns, 10 Hz rate | |
| Pulse energy | 62 mJ (16 J/cm²) for cheese, 50 mJ (13 J/cm²) for others | |
| Spot diameter | 700 | μm |
| Spectrometer | AvaSpec-Mini-VIS-OEM (Avantes, Netherlands) | |
| Spectral range | 350-600 | nm |
| Resolution | 0.33 | nm |

| | |
|---|---|
| Gate condition | 1.16 μs delay and 1.05 ms gate width |
| Stage control | 5 * 5 scanning in 6 * 6 mm² area |

Condition of hand-held instrument (Table 8)

TABLE 8

| Laser | Z-300 LIBS analyzer (SciAps Inc., US) | |
|---|---|---|
| Pulse width | 1-2 ns, 1064 nm beam, 10 Hz rate | |
| Pulse energy | 5 mJ (64 J/cm²) | |
| Spot diameter | 100 | μm |
| Ambient air | Argon gas | |
| Spectrometer | Contained 3 spectrometers | |
| Spectral range | 180-961 | nm |
| Resolution | 0.33 | nm |

| | |
|---|---|
| Gate condition | 0.65 μs delay and 1 ms gate width |
| Stage condition | 5 * 5 scanning in 1 * 1 mm² area |

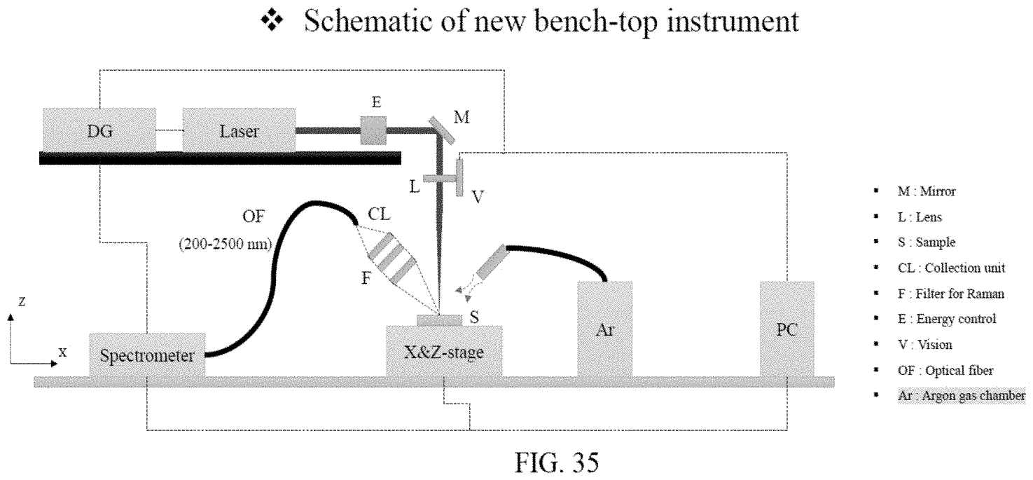

Schematic of new bench-top instrument (FIG. 35).
Condition of 2nd bench-top instrument (Table 9)

TABLE 9

| Laser | MicroJewel DPSS Nd: YAG 1064 nm laser (Quantum | |
|---|---|---|
| | Composers, USA) | |
| Pulse width | 6 ns, 10 Hz rate | |
| Pulse energy | 10 mJ (32 J/cm²) | |
| Spot diameter | 200 | μm |
| Spectrometer 1 | AvaSpec-Mini-VIS-OEM (Avantes, Netherlands) | |
| Spectral range | 350-600 | nm |
| Resolution | 0.33 | nm |
| Spectrometer 2 | Qmini VI (Broadcom, Netherlands) | |
| Spectral range | 370-750 | nm |
| Resolution | 0.33 | nm |

| | |
|---|---|
| Gate condition | 1.0 μs delay and 1.05 ms gate width |

Condition of 2nd bench-top instrument (for Raman) (Table 10)

TABLE 10

| Laser | MicroJewel DPSS Nd: YAG 1064 nm laser (Quantum | |
|---|---|---|
| | Composers, USA) | |
| Pulse energy | 0.1 mJ (0.05 GW/cm², too weak - 1 or 0.7 order | |
| Spectrometer 1 | AvaSpec-Mini-NIR (Avantes, Netherlands) | |
| Spectral range | 975-1700 | nm |
| Resolution | 6 | nm |
| Spectrometer 2 | AvaSpec-Mini-NIR-OEM (Avantes, Netherlands) | |
| Spectral range | 1100-1420 | nm |
| Resolution | 3 | nm |

| | |
|---|---|
| Gate condition | 0 μs delay and exposure time 50 ms |

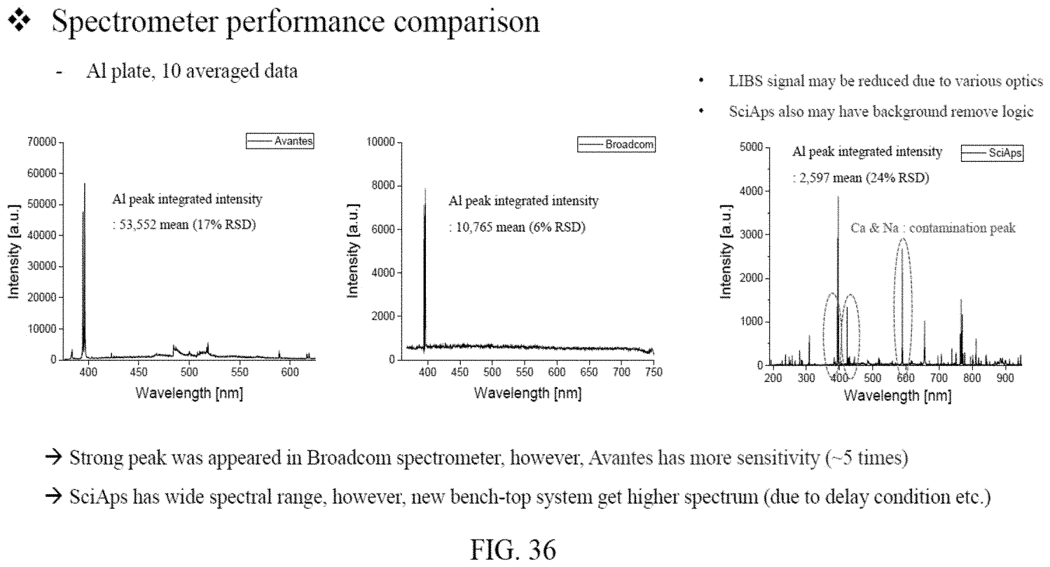

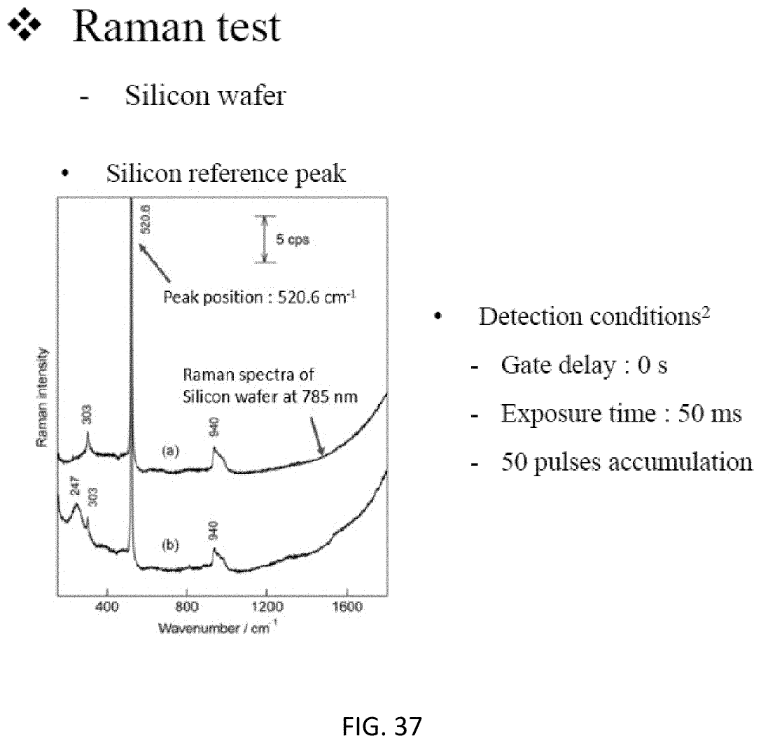

Spectrometer performance comparison (FIG. 36).
Raman test (FIG. 37)
Pre-processing of LIBS spectra data
1) Save raw data (Bench-top unit: 2048 pixels, Hand-held unit: 22855 pixels)
2) Normalization (reducing plasma fluctuation)
3) De-noise (reducing noise)
4) Filtering (remove having lower SNR data)

### 5) Standardization

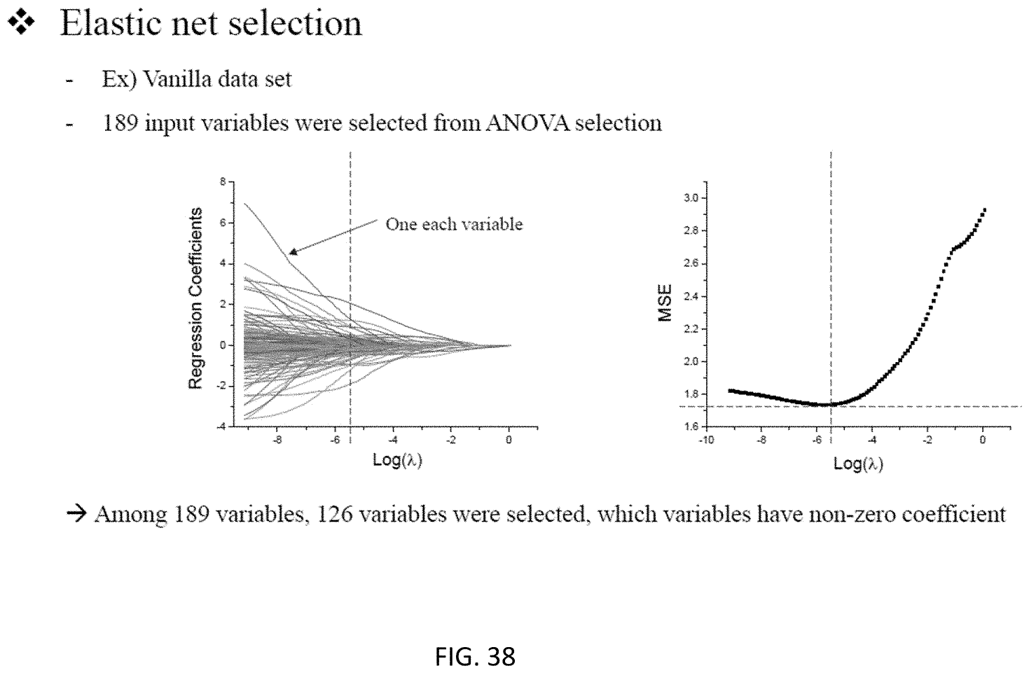

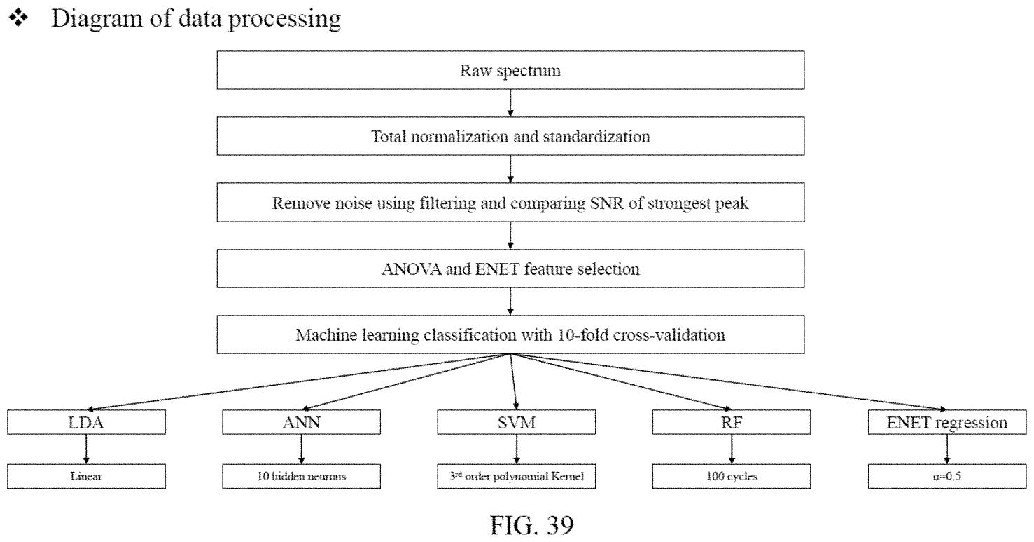

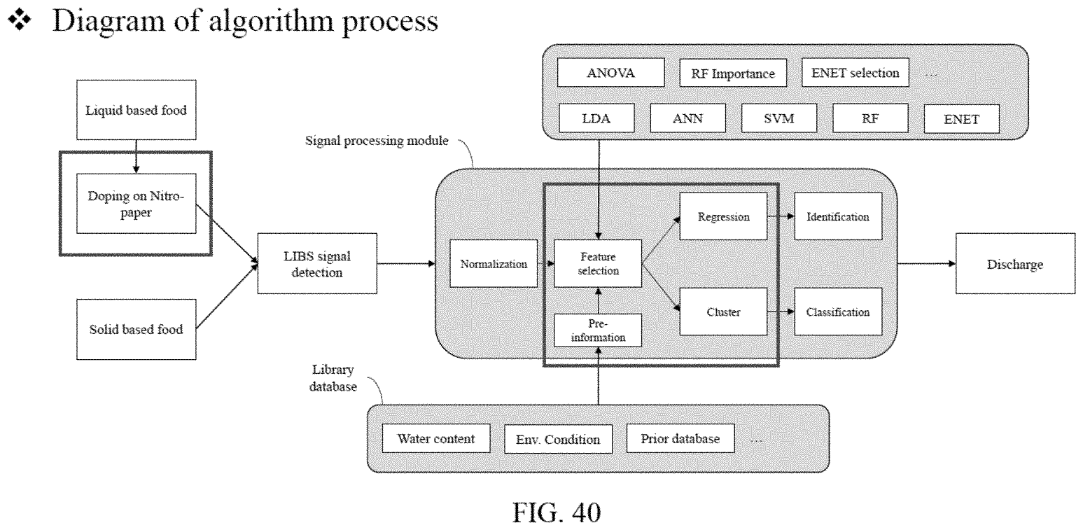

6) 10-fold cross-validation (divide 10 groups randomly)
Feature selection
1) Highest peak selection
2) PCA reduction or PCA coefficient
3) ANOVA analysis (determine the variances among different groups)
4) Importance variable selection from RF
Stepwise selection, Sequential selection, ENET selection
   Elastic net selection (FIG. 38).
   Various classifier
   1) LDA: Linear Discriminant Analysis (linear)
   2) ANN: Artificial Neural Networks (few numbers of hidden neurons)
   3) SVM: Support Vector Machine (3rd order polynomial Kernel function)
   4) RF: Random Forest (100 learning cycles)
   5) ENET regressions (sklearn model)
Statistics functions in Matlab and Python
   Diagram of data processing (FIG. 39).
   Diagram of algorithm process (FIG. 40).

LIBS Results

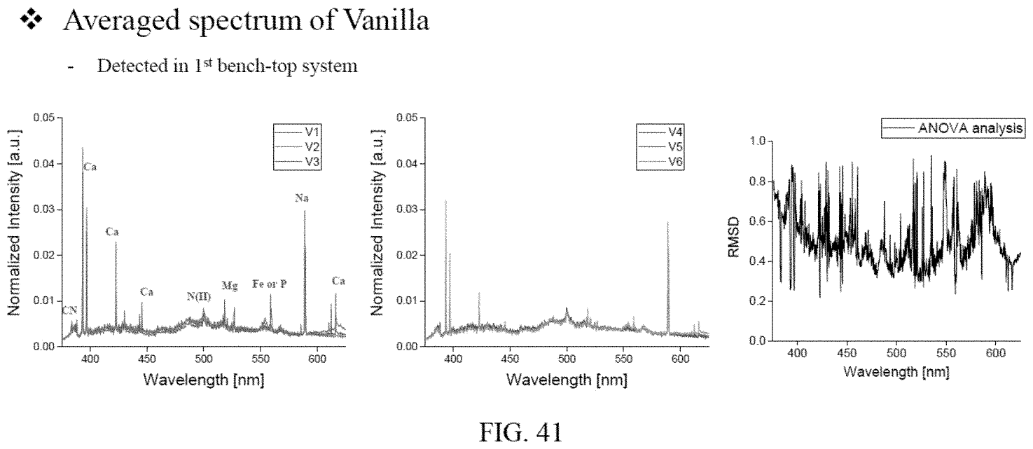

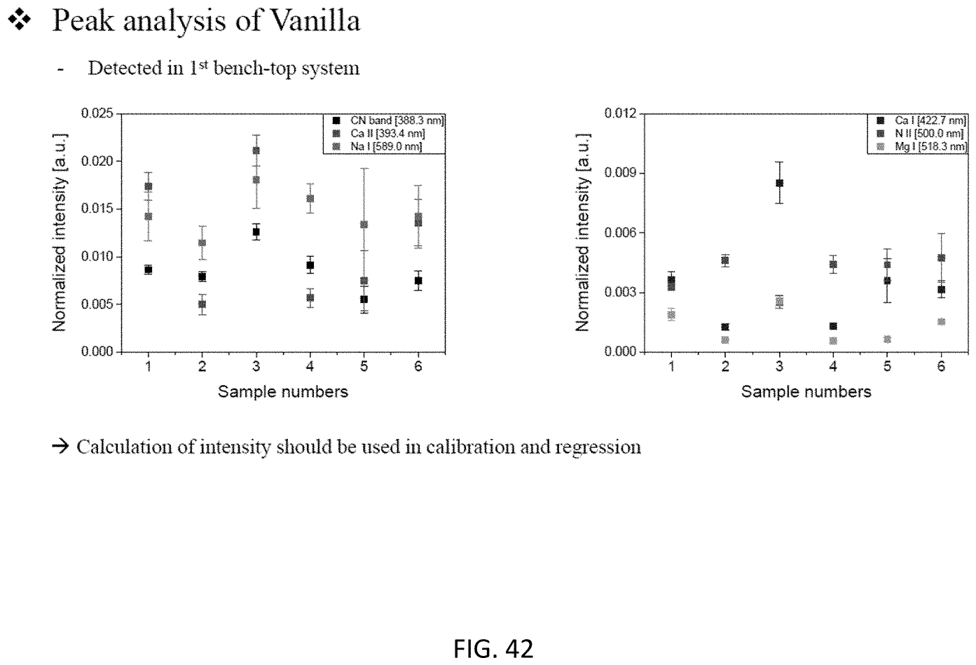

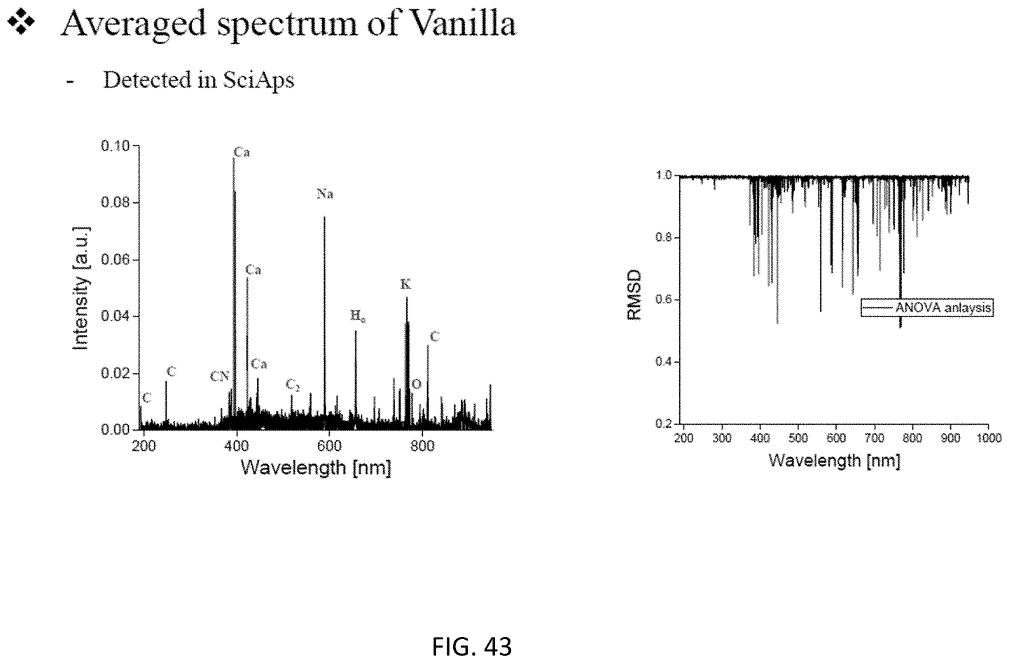

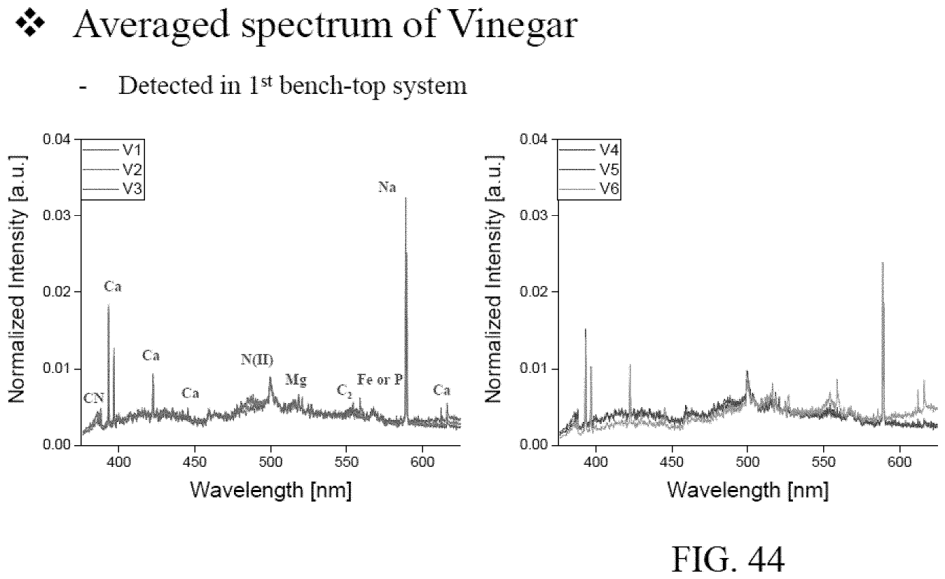

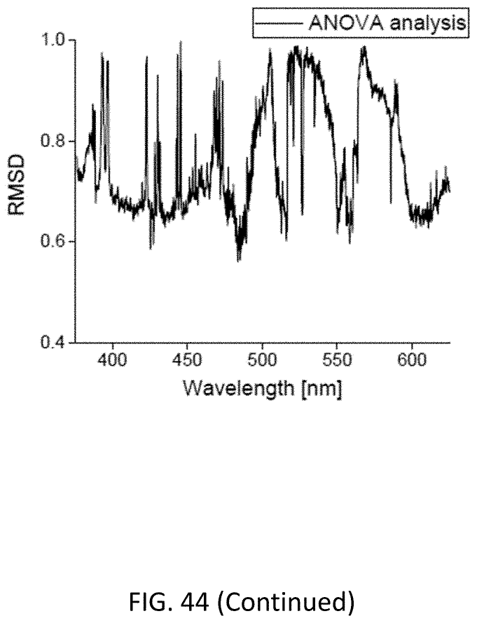

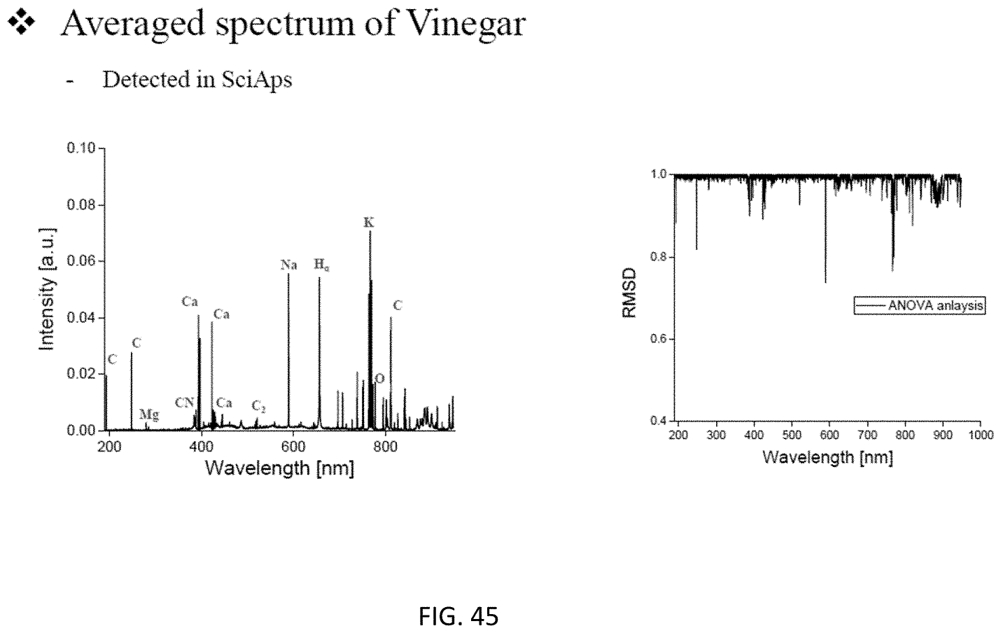

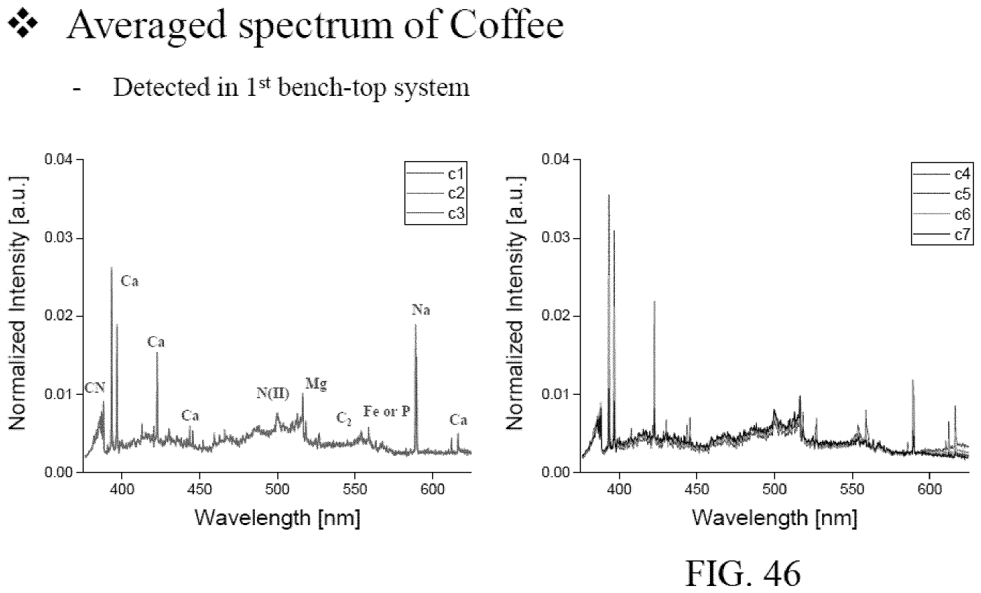

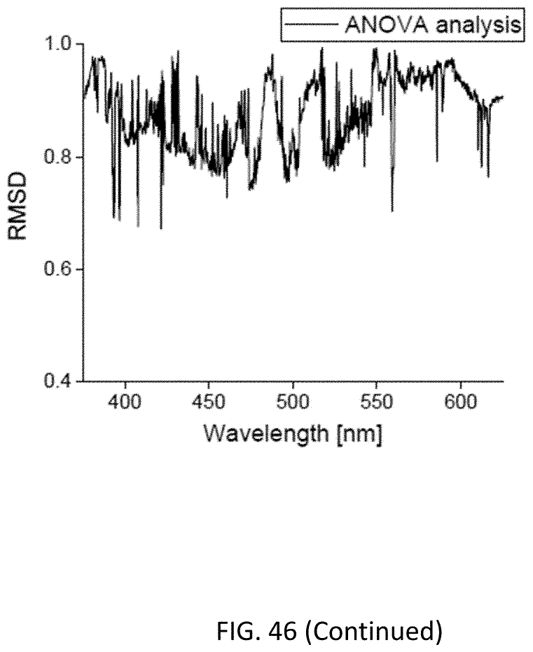

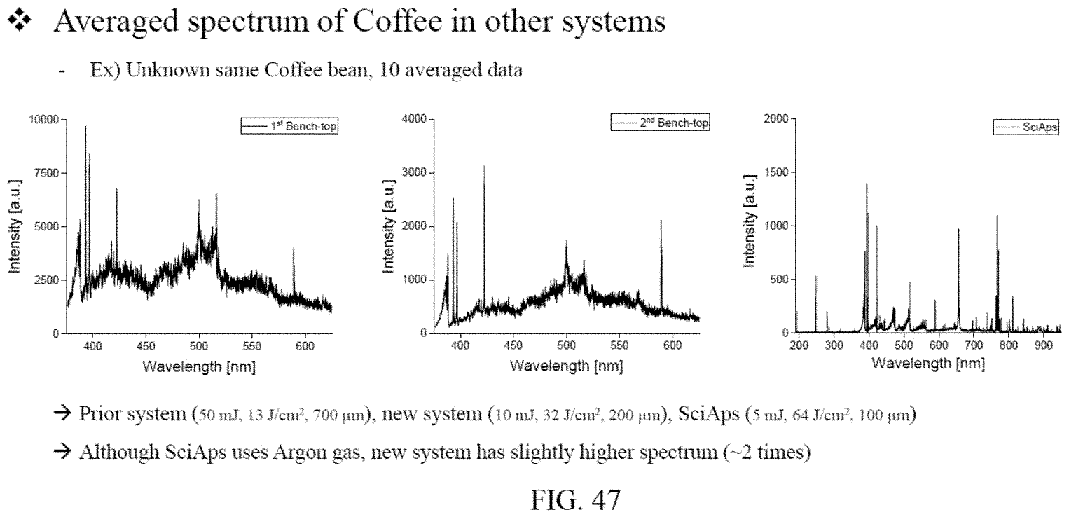

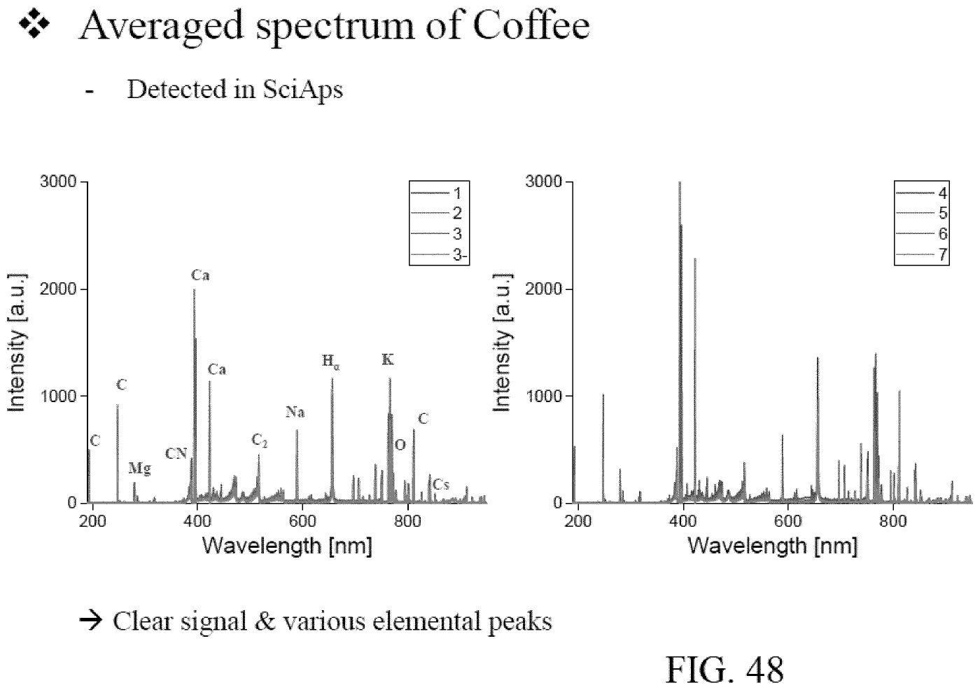

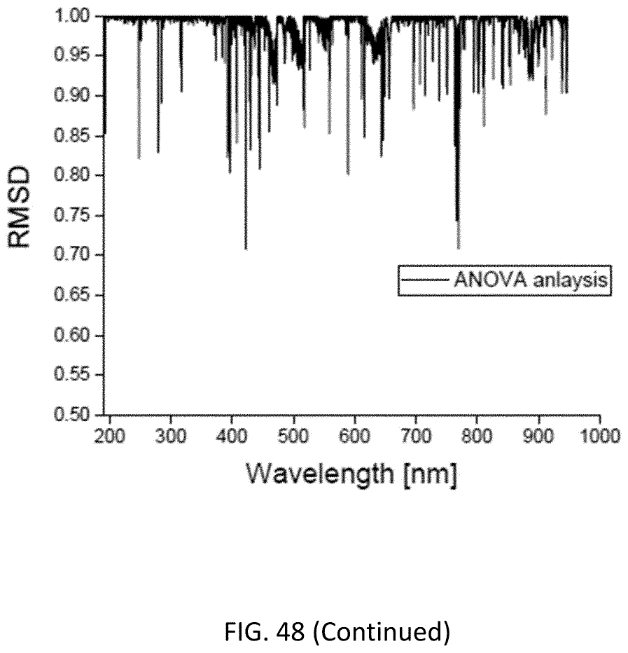

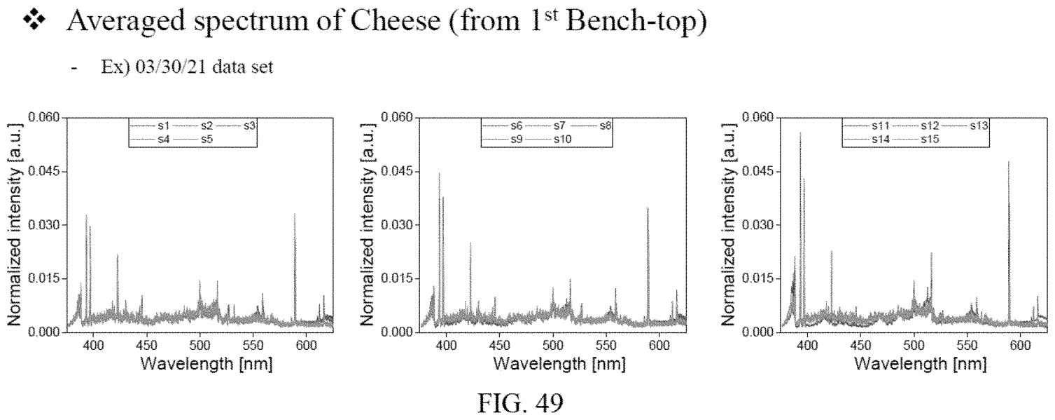

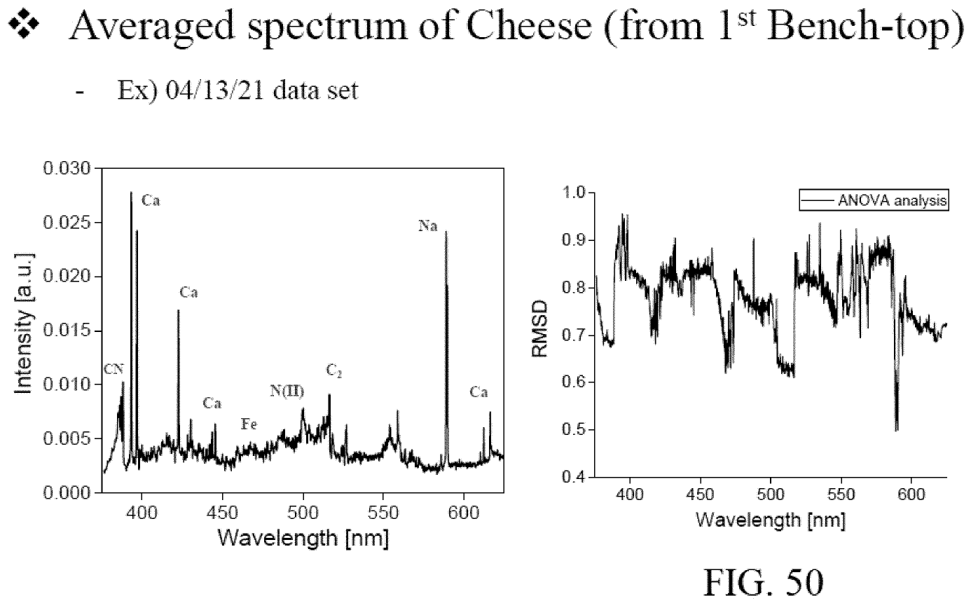

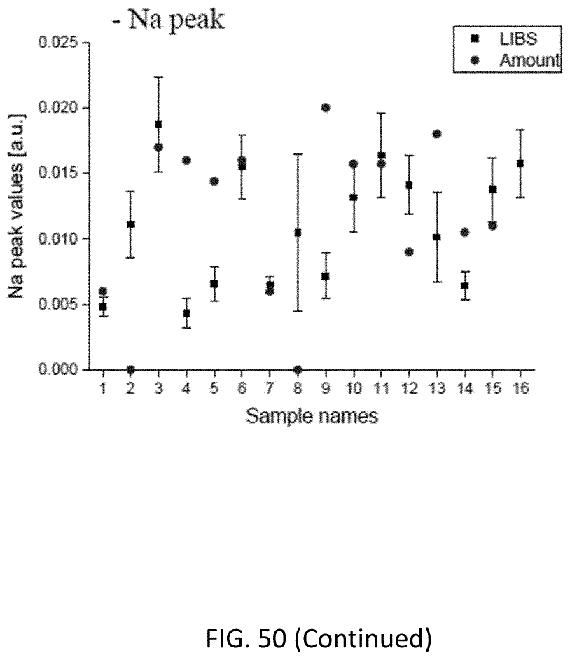

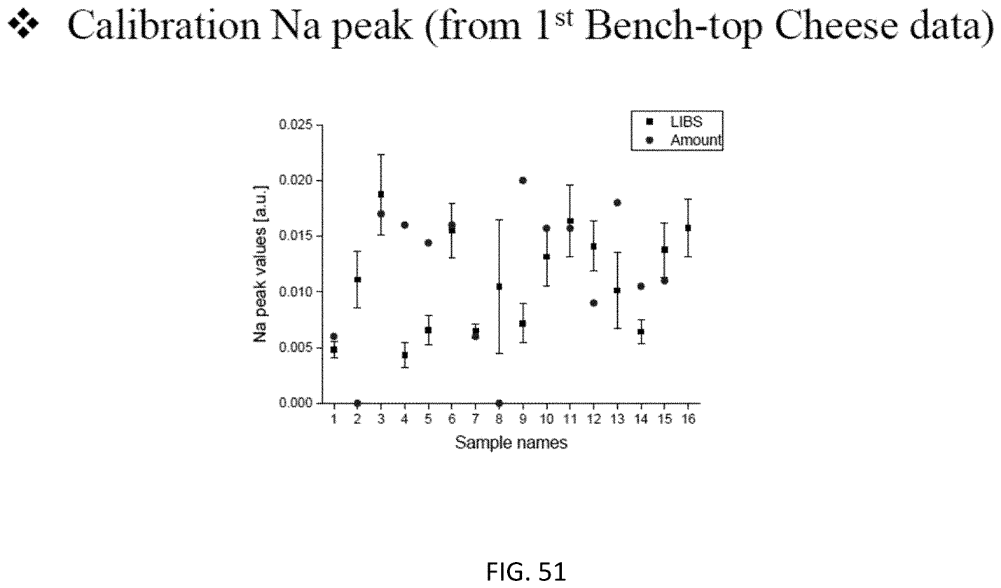

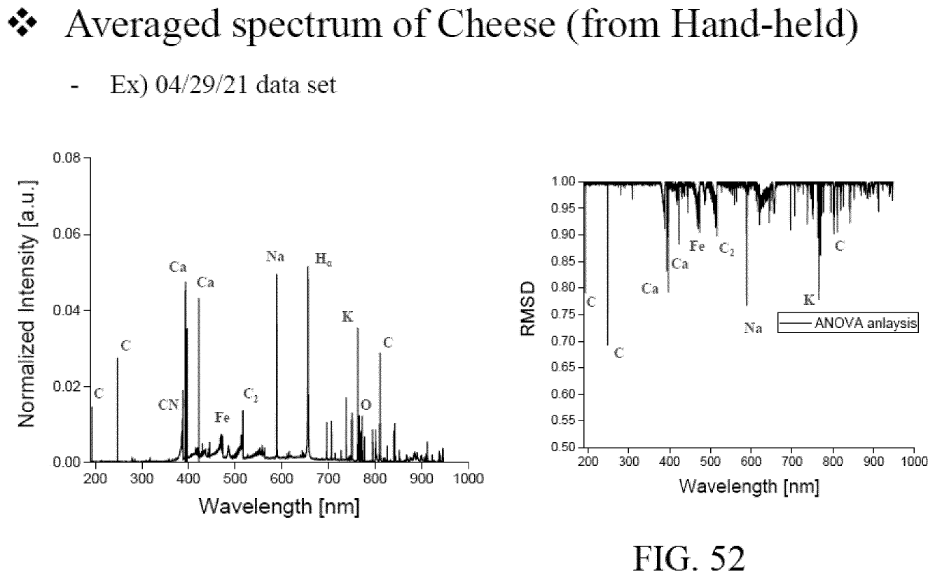

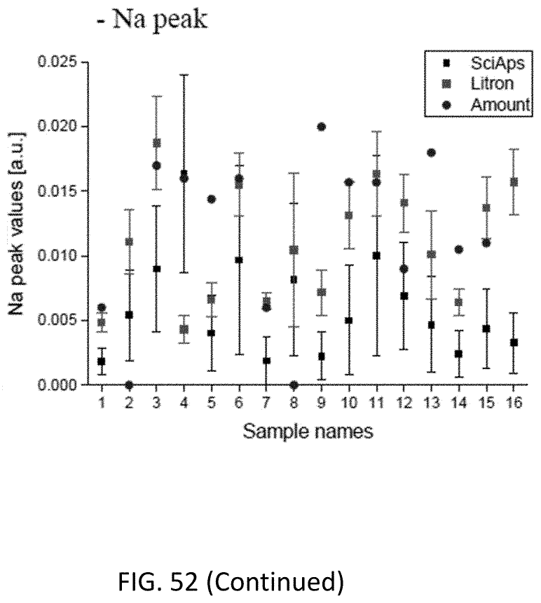

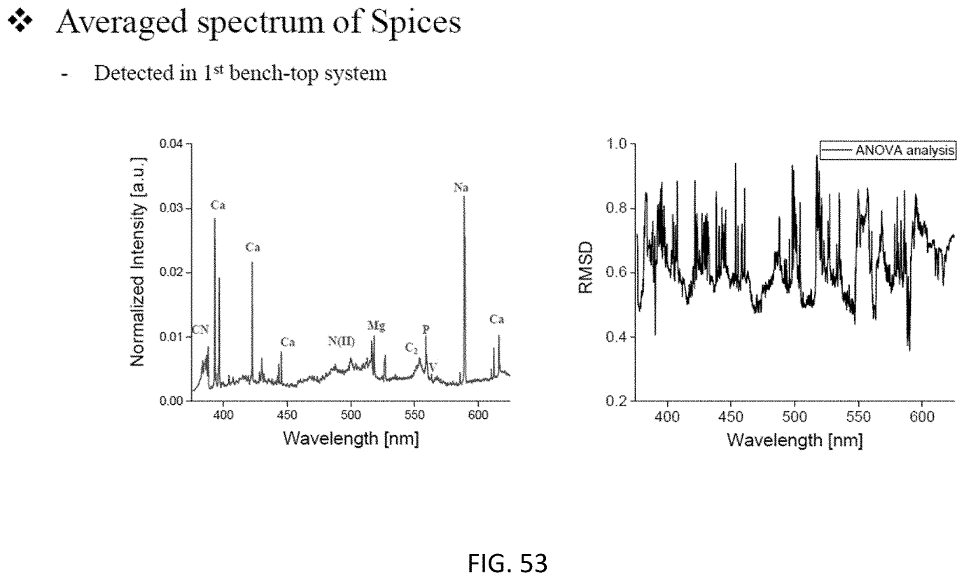

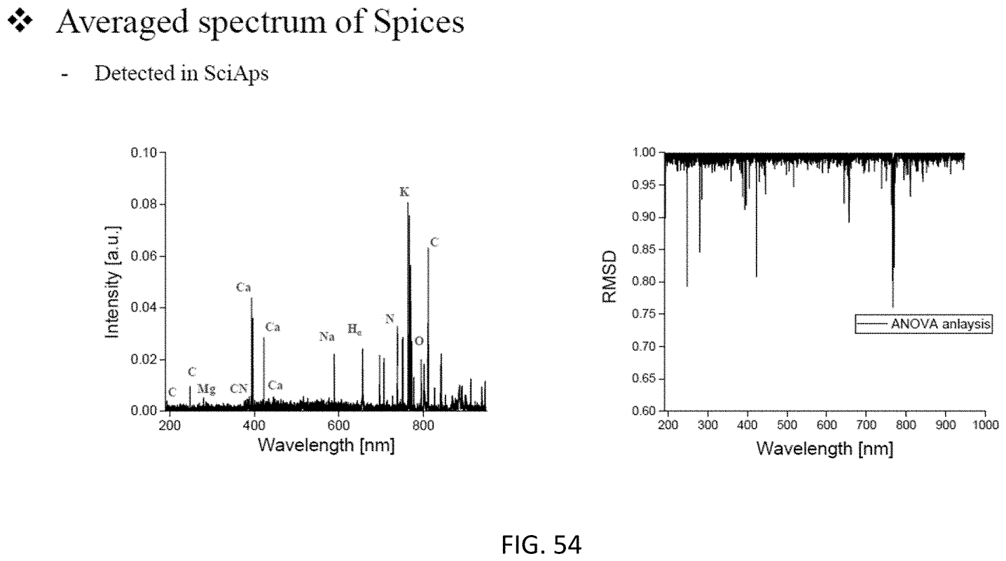

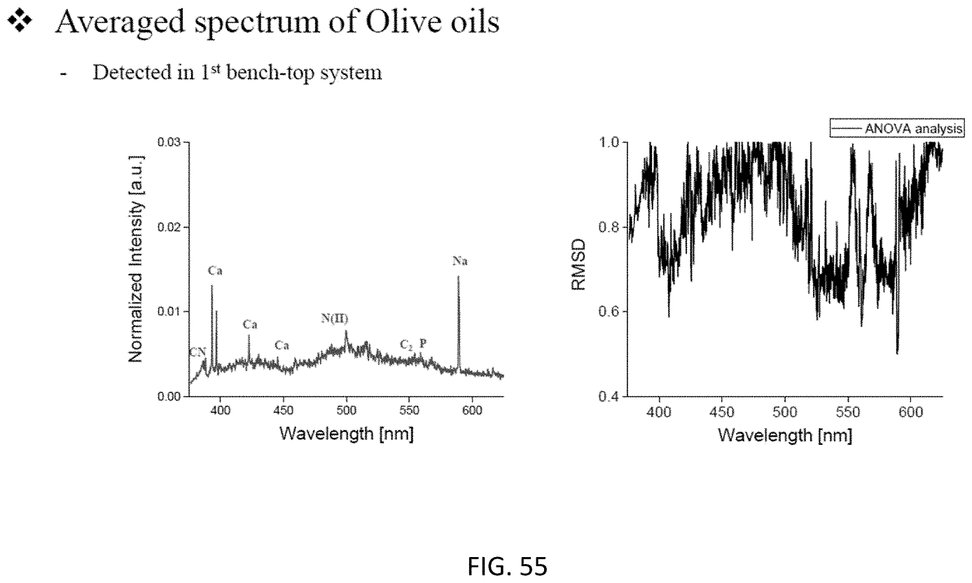

Averaged spectrum of Vanilla (FIG. 41).
Peak analysis of Vanilla (FIG. 42)
Averaged spectrum of Vanilla (FIG. 43).
Averaged spectrum of Vinegar (FIG. 44).
Averaged spectrum of Vinegar (FIG. 45).
Averaged spectrum of Coffee (FIG. 46).
Averaged spectrum of Coffee in other systems (FIG. 47).
Averaged spectrum of Coffee (FIG. 48).
Averaged spectrum of Cheese (from 1st Bench-top) (FIG. 49).
Averaged spectrum of Cheese (from 1st Bench-top) (FIG. 50).
Calibration Na peak (from 1st Bench-top Cheese data) (FIG. 51).
Averaged spectrum of Cheese (from Hand-held) (FIG. 52).
Averaged spectrum of Spices (FIG. 53).
Averaged spectrum of Spices (FIG. 54).
Averaged spectrum of Olive oils (FIG. 55).

Classification Results

### Vanilla (Tables 11-12)

Detected in 1st bench-top system
   Conditions: 126 input variables from ENET selection & 100 learning cycles

TABLE 11
Classification accuracy

| Classifier | Accuracy [%] |
|---|---|
| LDA | 90.5 |
| ANN | 92.6 |
| SVM | 93.4 |
| RF | 94.0 |
| ENET | 93.6 |

TABLE 12
• Confusion matrix from RF

| | V1 | V2 | V3 | V4 | V5 | V6 |
|---|---|---|---|---|---|---|
| V1 | 164 | 0 | 1 | 0 | 0 | 10 |
| V2 | 0 | 128 | 0 | 7 | 5 | 0 |
| V3 | 0 | 0 | 159 | 0 | 0 | 1 |
| V4 | 0 | 7 | 0 | 131 | 1 | 0 |
| V5 | 0 | 9 | 0 | 0 | 127 | 0 |
| V6 | 7 | 0 | 0 | 3 | 0 | 123 |

### Vanilla (Tables 13-14)

Detected in SciAps
   Conditions: 98 input variables from ENET selection & 10 hidden neurons

TABLE 13
Classification accuracy

| Classifier | Accuracy [%] |
|---|---|
| LDA | 95.6 |
| ANN | 98.0 |
| SVM | 86.3 |
| RF | 94.0 |
| ENET | 98.1 |

TABLE 14
• Confusion matrix from ANN

| | V1 | V2 | V3 | V4 | V5 | V6 |
|---|---|---|---|---|---|---|
| V1 | 143 | 0 | 3 | 1 | 0 | 0 |
| V2 | 2 | 140 | 0 | 0 | 0 | 0 |
| V3 | 0 | 0 | 141 | 0 | 1 | 1 |
| V4 | 1 | 0 | 0 | 140 | 0 | 0 |
| V5 | 0 | 2 | 1 | 2 | 63 | 0 |
| V6 | 0 | 0 | 2 | 0 | 0 | 146 |

### Vinegar (Tables 15-16)

Detected in 1st bench-top system
   Conditions: 107 input variables from ENET selection & 100 learning cycles

TABLE 15
Classification accuracy

| Classifier | Accuracy [%] |
|---|---|
| LDA | 72.3 |
| ANN | 79.8 |
| SVM | 82.9 |
| RF | 83.0 |
| ENET | 81.8 |

TABLE 16
• Confusion matrix from RF

| | V1 | V2 | V3 | V4 | V5 | V6 |
|---|---|---|---|---|---|---|
| V1 | 143 | 10 | 0 | 1 | 8 | 1 |
| V2 | 4 | 130 | 5 | 6 | 10 | 4 |
| V3 | 0 | 1 | 83 | 6 | 3 | 3 |
| V4 | 0 | 0 | 10 | 87 | 4 | 8 |
| V5 | 10 | 7 | 2 | 0 | 115 | 0 |
| V6 | 0 | 0 | 14 | 8 | 0 | 107 |

### Vinegar (Tables 17-18)

Detected in SciAps
   Conditions: 54 input variables from ENET selection & 10 hidden neurons

TABLE 17
Classification accuracy

| Classifier | Accuracy [%] |
|---|---|
| LDA | 83.9 |
| ANN | 84.9 |
| SVM | 80.7 |
| RF | 80.7 |
| ENET | 88.5 |

TABLE 18
• Confusion matrix from ANN

| | V1 | V2 | V3 | V4 | V5 | V6 |
|---|---|---|---|---|---|---|
| V1 | 139 | 6 | 1 | 1 | 0 | 0 |
| V2 | 6 | 137 | 0 | 2 | 2 | 3 |
| V3 | 0 | 0 | 44 | 2 | 13 | 5 |
| V4 | 0 | 0 | 2 | 56 | 3 | 1 |
| V5 | 0 | 0 | 10 | 13 | 70 | 8 |
| V6 | 1 | 2 | 10 | 0 | 7 | 103 |

### Coffee (Tables 19-20)

Detected in 1st bench-top system
   Conditions: 140 input variables from ENET selection & 10 hidden neurons in ANN

TABLE 19
Classification accuracy

| Classifier | Accuracy [%] |
|---|---|
| LDA | 72.8 |
| ANN | 77.6 |
| SVM | 68.0 |
| RF | 69.0 |
| ENET | 81.4 |

TABLE 20
• Confusion matrix from ANN

| | C1 | C2 | C3 | C4 | C5 | C6 | C7 |
|---|---|---|---|---|---|---|---|
| C1 | 77 | 5 | 2 | 0 | 6 | 1 | 0 |
| C2 | 2 | 45 | 12 | 0 | 2 | 3 | 3 |
| C3 | 1 | 14 | 50 | 0 | 1 | 2 | 1 |
| C4 | 0 | 0 | 0 | 74 | 3 | 1 | 0 |
| C5 | 0 | 2 | 0 | 0 | 43 | 8 | 14 |
| C6 | 2 | 0 | 0 | 0 | 0 | 43 | 0 |
| C7 | 1 | 0 | 0 | 0 | 12 | 0 | 42 |

### Coffee (Tables 21-22)

Detected in SciAps
   Conditions: 230 input variables from ENET selection & 10 hidden neurons in ANN
   Accuracy is improved implying that other elemental peaks (Mg, K, O etc.) are dominant for classification of coffee.

TABLE 21
Classification accuracy

| Classifier | Accuracy [%] |
|---|---|
| LDA | 91.0 |
| ANN | 93.7 |
| SVM | 79.9 |
| RF | 85.4 |
| ENET | 92.6 |

TABLE 22
• Confusion matrix from ANN

| | C1 | C2 | C3 | C4 | C5 | C6 | C7 |
|---|---|---|---|---|---|---|---|
| C1 | 172 | 8 | 0 | 0 | 4 | 3 | 0 |
| C2 | 11 | 179 | 0 | 1 | 0 | 3 | 2 |
| C3 | 0 | 0 | 190 | 0 | 0 | 1 | 1 |
| C4 | 0 | 0 | 0 | 178 | 2 | 2 | 0 |
| C5 | 7 | 0 | 0 | 0 | 171 | 8 | 0 |
| C6 | 1 | 3 | 0 | 4 | 10 | 171 | 4 |
| C7 | 1 | 2 | 1 | 0 | 3 | 2 | 179 |

Cheese from Bench-top instrument (Tables 23-241

TABLE 23
Classification accuracy

| Classifier | Accuracy [%] |
|---|---|
| LDA | 60.8 |
| ANN | 68.0 |
| SVM | 57.0 |
| RF | 65.0 |

Conditions 124 input variables from ENET selection & 16 hidden neurons

TABLE 24
Classification accuracy [10 classes]

| Classifier | Accuracy [%] |
|---|---|
| LDA | 73.9 |
| ANN | 76.0 |
| SVM | 72.8 |
| RF | 73.2 |

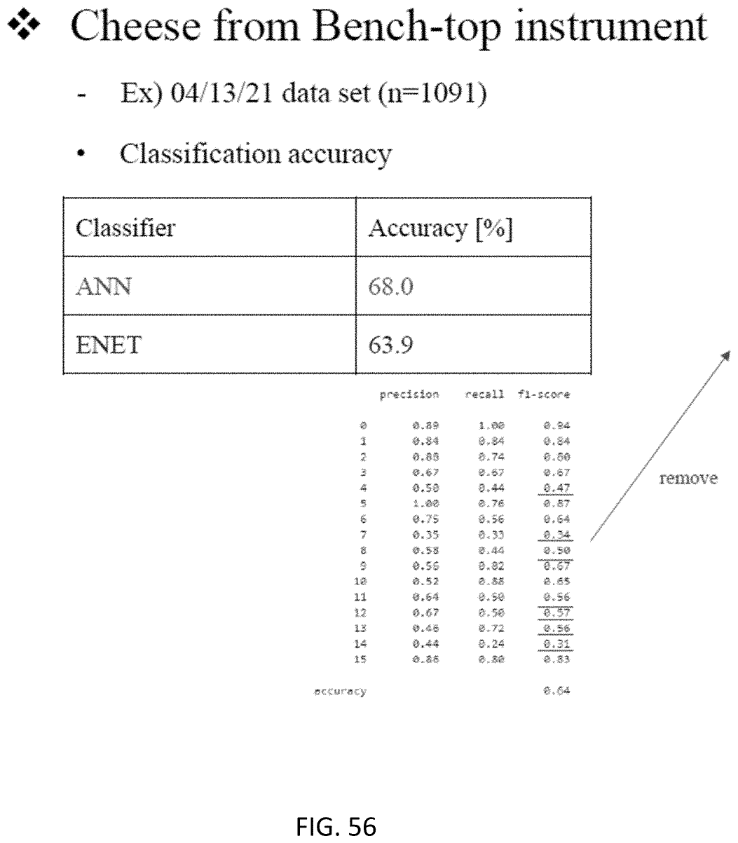

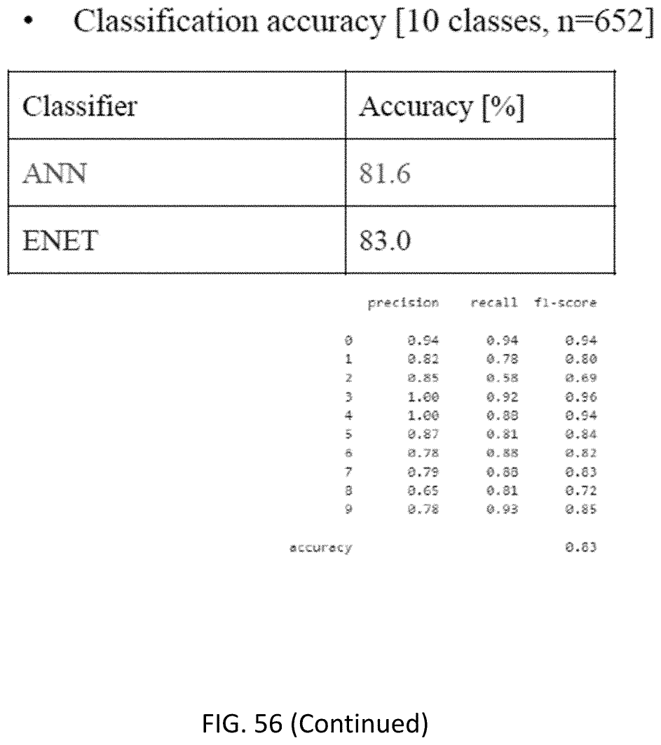

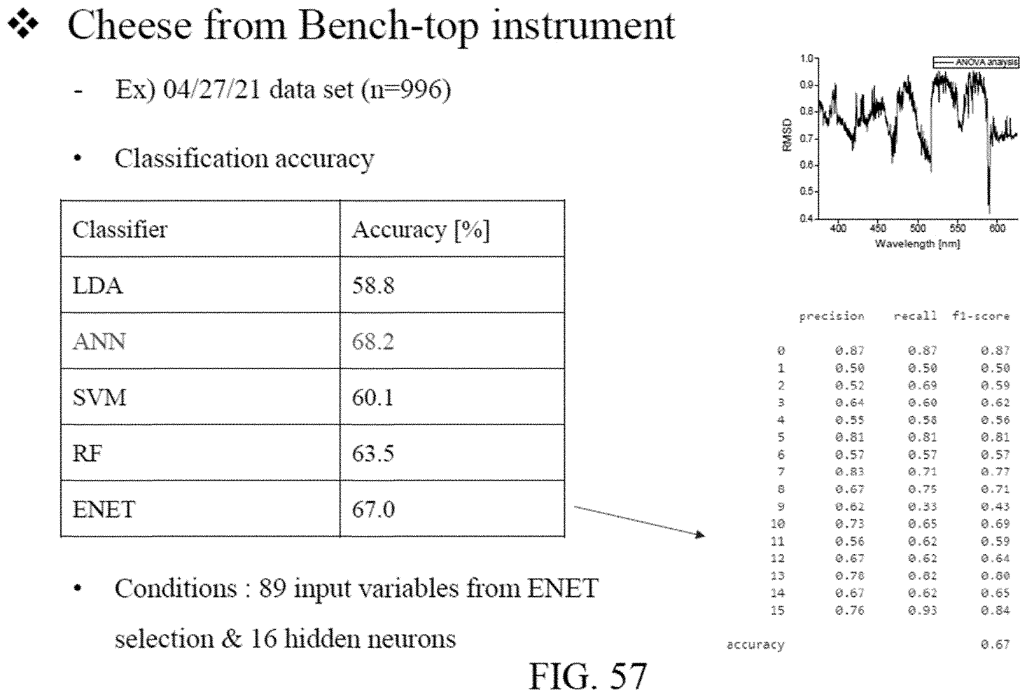

Conditions: 95 input variables from ENET selection &10 hidden neurons
   Cheese from Bench-top instrument (FIG. 56).
   Cheese from Bench-top instrument (FIG. 57).
   Cheese from Hand-held instrument (Table 25)

TABLE 25
Classification accuracy

| Classifier | Accuracy [%] |
|---|---|
| LDA | 62.3 |
| ANN | 66.0 |
| SVM | 43.9 |
| RF | 56.0 |
| ENET | |

Conditions as 124 input variables from ENET selection & 16 hidden neurons
   Cheese from Hand-held instrument (Tables 26-27 and FIG. 58).

TABLE 26
Classification accuracy

| Classifier | Accuracy [%] |
|---|---|
| LDA | 70.0 |
| ANN | 72.2 |
| SVM | 65.3 |
| RF | 67.3 |
| ENET | 71.3 |

Conditions: 135 input variables from ENET selection & 16 hidden neurons

TABLE 27
Classification accuracy [10 classes]

| Classifier | Accuracy [%] |
|---|---|
| LDA | 81.6 |
| ANN | 86.2 |
| SVM | 75.5 |
| RF | 78.7 |
| ENET | 82.1 |

Conditions input variables from ENET selection & 10 hidden neurons
   Number of types: 10 (reduced other similar types)
Number of spectra: n=653 (Bench-top)//n=825 (SciAps) (Tables 28-29)

TABLE 28
• Bench-top

| | Accuracy | Sensitivity | Specificity | PPV | NPV |
|---|---|---|---|---|---|
| LDA | 83.6 | 83.5 | 98.2 | 84.2 | 98.2 |
| ANN | 85.5 | 85.6 | 98.4 | 86.0 | 98.4 |
| SVM | 82.1 | 82.0 | 98.0 | 82.9 | 98.0 |
| RF | 78.1 | 78.6 | 97.6 | 78.8 | 97.6 |
| ENET | 84.1 | 83.7 | 98.2 | 83.9 | 98.2 |

TABLE 29
• SciAps

| | Accuracy | Sensitivity | Specificity | PPV | NPV |
|---|---|---|---|---|---|
| LDA | 81.6 | 80.8 | 98.0 | 81.6 | 98.0 |
| ANN | 86.2 | 85.7 | 98.5 | 85.7 | 98.6 |
| SVM | 75.5 | 75.2 | 97.3 | 76.7 | 97.4 |
| RF | 78.7 | 77.9 | 97.6 | 78.3 | 97.6 |
| ENET | 82.1 | 82.6 | 98.0 | 81.6 | 98.0 |

Number of types: 16
Number of spectra: n=285 (Bench-top)//n=441 (SciAps) (Tables 30-31)

TABLE 30
• Bench-top

| | Accuracy | Sensitivity | Specificity | PPV | NPV |
|---|---|---|---|---|---|
| LDA | 74.0 | 74.0 | 98.3 | 75.5 | 98.3 |
| ANN | 86.2 | 86.2 | 99.1 | 86.7 | 99.1 |
| SVM | 74.5 | 74.4 | 98.3 | 76.7 | 98.3 |
| RF | 80.4 | 80.5 | 98.7 | 81.0 | 98.7 |
| ENET | 82.7 | 82.9 | 98.9 | 84.7 | 98.9 |

Conditions: 108 input variables from ENET selection & 10 hidden neurons

TABLE 31
SciAps

| | Accuracy | Sensitivity | Specificity | PPV | NPV |
|---|---|---|---|---|---|
| LDA | 77.1 | 76.8 | 98.5 | 77.4 | 98.5 |
| ANN | 84.1 | 84.0 | 98.9 | 84.8 | 98.9 |
| SVM | 79.1 | 79.7 | 98.6 | 82.0 | 98.6 |
| RF | 80.4 | 79.8 | 98.7 | 80.1 | 98.7 |
| ENET | 84.1 | 84.9 | 98.9 | 85.8 | 98.9 |

Conditions: 150 input variables from ENET selection & 10 hidden neurons

### Spices (Table 32)

Detected in 1st bench-top system
   Conditions: 00 input variables from ENET selection & 10 hidden neurons

TABLE 32
Classification accuracy

| Classifier | Accuracy [%] |
|---|---|
| LDA | 96.9 |
| ANN | 97.6 |
| SVM | 94.4 |
| RF | 97.1 |
| ENET | 98.6 |

### Spices (Tables 33-34)

Detected in SciAps
   Conditions: 40 input variables from ENET selection & 10 hidden neurons

TABLE 33
Classification accuracy

| Classifier | Accuracy [%] |
|---|---|
| LDA | 75.3 |
| ANN | 80.6 |
| SVM | 74.9 |
| RF | 79.6 |
| ENET | 78.8 |

TABLE 34
• Confusion matrix from RF

| | V1 | V2 | V3 | V4 | V5 | V6 | V7 | V8 |
|---|---|---|---|---|---|---|---|---|
| V1 | 97 | 0 | 13 | 4 | 7 | 0 | 1 | 2 |
| V2 | 2 | 26 | 1 | 0 | 6 | 2 | 2 | 1 |
| V3 | 0 | 0 | 110 | 5 | 3 | 2 | 1 | 1 |
| V4 | 1 | 2 | 1 | 132 | 1 | 1 | 0 | 1 |
| V5 | 7 | 5 | 0 | 2 | 58 | 9 | 0 | 0 |
| V6 | 0 | 5 | 0 | 0 | 7 | 47 | 4 | 9 |
| V7 | 0 | 0 | 0 | 0 | 4 | 0 | 81 | 0 |
| V8 | 2 | 2 | 0 | 0 | 0 | 15 | 18 | 69 |

Olive oils (Tables 35-36)
Detected in 1st bench-top system
   Conditions: 28 input variables from ENET selection & 10 hidden neurons

TABLE 35
Classification accuracy

| Classifier | Accuracy [%] |
|---|---|
| LDA | 84.2 |
| ANN | 86.1 |
| SVM | 84.2 |
| RF | 82.2 |
| ENET | 84.6 |

TABLE 36
Confusion matrix from ANN

| | V1 | V2 | V3 |
|---|---|---|---|
| V1 | 15 | 0 | 0 |
| V2 | 10 | 24 | 2 |
| V3 | 0 | 2 | 48 |

What is claimed is:

1. A method for sample classification, the method comprising:
obtaining a plurality of known samples;
performing a spectroscopic analysis on the plurality of known samples to obtain an emission spectrum from each of the plurality of known samples; and
processing data from the emission spectra to identify a spectral fingerprint for each of the plurality of known samples using automated feature selection.

2. The method of claim 1, wherein the sample is a food sample.

3. The method of claim 2, wherein the sample is selected from the group consisting of cheese, coffee, olive oil, vanilla extract, and spices.

4. The method of claim 1, wherein the spectroscopic analysis performed comprises laser-induced breakdown spectroscopy (LIBS).

5. The method of claim 1, wherein the automated feature selection comprises machine learning classification selected from the group consisting of linear discriminant analysis (LDA), an artificial neural network (ANN), support vector machine (SVM), random forest (RF), and elastic net (ENET) regression.

6. The method of claim 1, wherein one or more of the plurality of known samples is a liquid sample, the method further comprising depositing the liquid sample on a cellulose strip before performing the spectroscopic analysis.

7. The method of claim 1, further comprising:
obtaining a test sample;
performing a spectroscopic analysis on the test sample to obtain an emission spectrum from the test sample; and
authenticating the test sample by comparing the emission spectra for the test sample to an expected spectral fingerprint from the spectral fingerprints for the plurality of known samples.

8. The method of claim 1, wherein the processing step further comprises:
spectral baseline adjustment and correction;
filtering and denoising;
normalization;
univariate feature filtering employing generalized linear models;
multivariate feature selection and classification using regularized regression; and
classification using one or more machine learning methodologies.

9. The method of claim 8, wherein the one or more machine learning methodologies comprise an elastic-net feature selection model with combined LASSO and ridge penalties.

10. The method of claim 1, further comprising providing one or more additional data points for the plurality of known samples, wherein the processing the data from the emission spectra step includes analysis the one or more additional data points to identify a fingerprint for each the plurality of known samples comprising features selected from among the one or more additional data points along with the spectral fingerprint.

11. The method of claim 10, wherein the one or more additional data points are selected from the group consisting of spectra from one or more different spectroscopic technique and data from one or more biophysical testing methods.

12. A method for detecting molecules in a sample, the method comprising:
providing a sample comprising a target molecule;
applying the sample to a porous substrate comprising metal-conjugated capture molecules specific to the target molecule;
wicking the sample along the porous substrate to concentrate target molecule bound capture molecules at a test region on the porous substrate and to concentrate unbound capture molecules at a control region on the porous substrate;
performing a spectroscopic analysis on the test region and the control region to detect a concentration of the metal-conjugated capture molecules therein; and
confirming presence of the target molecule in the sample based on detection of the metal-conjugated capture molecules in both the test region and the control region.

13. The method of claim 12, wherein the metal-conjugated capture molecule comprises a gold nanoparticle-conjugated antibody specific to the target molecule.

14. The method of claim 12, wherein the metal-conjugated capture molecule comprises a lanthanide-conjugated antibody specific to the target molecule.

15. The method of claim 12, wherein the molecule comprises a cytokine.

16. The method of claim 15, wherein the cytokine comprises interleukin 6 (IL-6).

17. The method of claim 15, wherein the sample is obtained from a patient at risk of a cytokine storm.

18. The method of claim 12, further comprising quantifying an amount of metal-conjugated capture molecules concentrated at the test region using the spectroscopic analysis.

19. The method of claim 12, wherein the spectroscopy analysis comprises laser-induced breakdown spectroscopy (LIBS).

20. The method of claim 12, wherein the porous substrate is a nitrocellulose membrane.

21. The method of claim 12, wherein the confirming presence step occurs 15 minutes or less after the applying step.
